(19) United States
(12) Patent Application Publication
(10) Pub. No.: US 2024/0425848 A1
(43) Pub. Date: Dec. 26, 2024

(54) ENGINEERED TRANSPOSASE AND USES THEREOF

(71) Applicants: Ospedale San Raffaele S.r..l., Milan (IT); Fondazione Centro San Raffaele, Milan (IT); Istituto Europeo Di Oncologia S.r.l., Milan (IT)

(72) Inventors: Giovanni Tonon, Milan (IT); Davide Cittaro, Milan (IT); Martina Tedesco, Milan (IT); Francesca Giannese, Milan (IT); Dejan Lazarevic, Milan (IT); Sebastiano Pasqualato, Milan (IT)

(21) Appl. No.: 18/274,079

(22) PCT Filed: Jul. 22, 2022
(86) PCT No.: PCT/EP2022/052915

§ 371 Date: Jul. 25, 2023

(30) Foreign Application Priority Data
Feb. 5, 2021 (GB) 2101656.3
Jul. 7, 2021 (GB) 2109803.3

(51) Int. Cl. C12N 15/10; C12N 9/12

(52) U.S. Cl. CPC C12N 15/1065; C12N 9/1241

(57) ABSTRACT

The present invention relates to an engineered transposase comprising a transposase operably linked to a polypeptide that binds to a component of heterochromatin. The present invention further relates to an engineered transposome complex comprising an oligonucleotide and an engineered transposase according to the invention. The present invention also relates to methods and uses of the engineered transposase of the invention and engineered transposome of the invention for making a DNA sequence library or libraries and for DNA sequencing.

### FIELD OF THE INVENTION

The present invention relates to the field of genomic and epigenomic analysis. More specifically, the present invention relates to an engineered transposase and an engineered transposome to target specific regions of chromatin. The present invention also relates to methods for genomic and/or epigenomic analysis and uses of the engineered transposase and/or engineered transposome of the invention for genomic and/or epigenomic analysis.

### BACKGROUND TO THE INVENTION

Epigenetic modifications are heritable phenotype changes that do not result from alteration of the DNA sequence itself. Epigenetic mechanisms are highly conserved throughout eukaryotes. Examples of epigenetic modifications include histone modification and DNA methylation, each of which alters gene expression without changing the underlying DNA sequence. In particular, histone modification alters local chromatin structure and thereby gene expression.

Several human diseases are the result of disrupted epigenetics impinging on underlying genetic lesions. A case in point is represented by cancer. Cancers are characterized by extensive inter-patient and intra-tumour heterogeneity, down to the single cell level. This fuels clonal evolution, leading to treatment resistance, both primary and acquired, which is the leading cause of death for cancer patients. Despite extensive studies, the mechanisms underlying this resistance are still largely unknown both for standard chemotherapeutic regimens and for the recently introduced immunotherapies. Increasingly detailed analysis of cancer genomes, before and after treatment, have so far failed to identify genetic causes, such as the acquisition of somatic mutations or copy number aberrations, which could explain the ensuing refractoriness to therapeutic regimens. Growing evidence points to epigenetic traits as crucially important in driving the acquisition of resistance towards anticancer regimens. This suggests that only a comprehensive assessment of both genetic changes of the cancer genome and the concomitant chromatin remodelling events ensuing after treatment could finally provide the insights required to tackle this pressing unmet clinical need. Additionally, given the rampant heterogeneity that is present within cancer cell populations, single-cell approaches are emerging as truly revolutionary tools to reliably and comprehensively capture cancer heterogeneity and inform on treatment resistance mechanisms.

Next-generation sequencing (NGS) has transformed genomic research by reducing turnaround time and cost. Library construction plays an important role for high-throughput NGS. A plethora of library construction methods have been developed, including the traditional ligation-based methods and the more recently developed transposase-based Nextera method.

The transposase-based Nextera approach employs an in vitro transposition reaction, using a transposome complex formed of a transposase Tn5 and a free transposon end that contains a transposase recognition site mosaic end (ME) and a sequencing adaptor (which may be a sequencing primer). When the transposome complex is incubated with target double-stranded DNA (dsDNA), the target dsDNA undergoes tagmentation by the transposase. Thus, the target dsDNA is fragmented and the transposon (including the ME and the sequencing primer) is covalently attached to the 5′ end of the target dsDNA fragment, resulting in a sequencing-ready DNA library. Nextera libraries can also incorporate tagging sequences (also termed barcodes), enabling multiplexed sequencing in a single run.

Whilst significant improvements have been made in genome sequencing approaches, methodologies currently used for sequencing of chromatin fragments suffer from various limitations.

Conventional chromatin immunoprecipitation with sequencing (ChIP-seq) is a complex, time consuming and multistep process involving crosslinking of DNA and protein in live cells, extraction followed by shearing of crosslinked material, immunoprecipitation of crosslinked DNA-protein complexes (by antibody binding of the protein of interest), reverse crosslinking, and the sequencing of the resulting DNA molecules. Thus, ChIP-seq and its variations involve performing DNA sequence analysis on the fraction of DNA isolated by immunoprecipitation with antibodies specific to the protein of interest, which is directly or indirectly associated with DNA. These methodologies suffer from low signals, high backgrounds, epitope masking due to cross-linking, low yields which require large numbers of cells, limitations associated with efficient immunocapture of protein-associated DNA, and technical challenges associated with the use of antibodies. In particular, ChIP-seq and other antibody-based approaches are limited to a single library per immunoprecipitation, i.e. these methods are not suitable for multiplex sequencing analysis of different epigenetic markers.

ChIP-seq and Nextera sequencing have also been integrated in an approach termed transposase assisted chromatin immunoprecipitation (TAM-ChIP). This approach combines the antibody-mediated targeting of chromatin immunoprecipitation with the ability of Tn5 to tagment DNA, leading to chromatin fragmentation and tagging of the chromatin surrounding the antibody binding site. In this process, a transposase is conjugated to an antibody such that antibody-directed tagmentation of DNA by the transposase occurs following antibody binding to the target molecule. This approach relies on antibodies, which pose technical challenges.

Recently, a method for determining chromatin accessibility has been developed, termed Assay for Transposase-Accessible Chromatin using sequencing (ATACseq). This method uses transposases to probe accessible chromatin. Transposases allow for the fragmenting and then sequencing of native accessible chromatin in bulk (ATACseq), as well as at the single-cell level (scATAC-seq). This approach is providing key insights on the cellular status of open chromatin. However, the epigenetic modifications of large portions of the genome which exert essential roles in cellular physiology are excluded from this analysis.

Hence, while recent efforts have succeeded in surveying open chromatin, the high-throughput, single-cell assessment of genomic and epigenetic landscapes remains challenging.

Thus, there is a significant need in the art for a tool which comprehensively audits, for example at the single cell level, both the genomic and the epigenetic landscape.

### SUMMARY OF THE INVENTION

The present inventors have developed engineered transposases which have been redirected to bind to a different component of chromatin compared to the corresponding wild type transposase. This permits the analysis of chromatin modifications which were previously excluded from sequencing analyses.

In addition, the present inventors have devised a genomic and epigenetic approach, termed “genome and epigenome by transposases sequencing” (GET-seq), which can be performed at the single-cell level (scGET-seq), that may exploit such engineered transposases to comprehensively probe open and closed chromatin, concomitantly recording the underlying genomic sequences. Hence, a comprehensive epigenetic assessment of heterochromatin is achieved. Additionally, building upon the differential enrichment between closed and open chromatin, the present inventors devised a method using scGET-seq, termed “Chromatin Velocity”, which identifies the trajectories of epigenetic modifications at the single-cell level. Thus, GET-seq, and in particular, scGET-seq, may illuminate the dynamic and evolving genomic and epigenetic landscapes of single cell populations in physiology and human diseases.

Furthermore, the present inventors have devised a multiomics approach (i.e. an approach which combines multiple omics technologies), termed GET²-seq, which can be performed at the single-cell level (scGET²-seq), that may exploit the engineered transposases described herein to comprehensively probe open and closed chromatin, concomitantly recording the underlying genomic sequences while simultaneously capturing RNA. Hence, a comprehensive genomic, epigenomic and transcriptomic approach may be achieved. Thus, GET²-seq, and in particular, scGET²-seq, may illuminate the dynamic and evolving genomic, epigenetic and transcriptomic landscapes of single cell populations in physiology and human diseases.

The methods of the invention significantly improve the principle techniques currently used for sequencing of chromatin fragments, such as for epigenetic analysis, including Nextera (transposon-based), ATAC-seq (transposon-based), ChIP and TAM-ChIP. In particular, existing methodologies may not be suitable for single cell analysis, require extraction and optionally fragmentation of genomic DNA, exclude epigenetic modifications of large portions of the genome and/or rely on antibodies, which pose technical challenges. The methods of the invention permit multiplex sequencing analysis and is less time-consuming, i.e. more rapid and efficient, since they do not require steps such as histone-DNA crosslinking, chromatin shearing and de-crosslinking. Further, the GET²-seq method permits simultaneous genomic, epigenomic and transcriptiomic profiling.

Advantages of the methods of the invention over conventional techniques include the following:
the need for antibodies is eliminated, thus providing a more efficient and robust process;
the insertion of the oligonucleotide to the DNA by the transposase reduces the input DNA requirement;
the need for pre-processing of genetic material is eliminated (i.e. the method can be performed on intact cells), providing a more efficient and cost effective process;
the methods of the invention may be applicable to a broader range of chromatin targets which were previously excluded due to the limited targeting of the available transposases and/or the lack of suitable antibodies for certain targets;
the methods of the invention are applicable to single cell analysis;
the methods of the invention are applicable to multiplexed sequencing applications; the methods of the invention permit simultaneous and dynamic profiling of both accessible and compacted chromatin, i.e. simultaneous and dynamic genomic and epigenetic analysis, even at the single cell level; and.
the multiomics methods of the invention achieve simultaneous and dynamic profiling of the chromatin conformation state (euchromatin and heterochromatin) and capture of RNA, e.g. simultaneous and dynamic genomic, epigenomic and transcriptomic profiling, even at the single cell level.

Accordingly, in one aspect, the invention provides a method for making a DNA sequence library or libraries comprising the steps:
a) providing a sample comprising genomic DNA;
b) adding at least one engineered transposome complex according to the invention;
c) optionally amplifying tagged DNA; and
d) optionally isolating the amplified DNA.

In some embodiments, the method further comprises the step of sequencing tagged DNA, the amplified DNA or the isolated DNA.

In a further aspect, the invention provides a method for DNA sequencing comprising the steps:
a) providing a sample comprising genomic DNA;
b) adding at least one engineered transposome complex according to the invention;
c) optionally amplifying tagged DNA;
d) optionally isolating the amplified DNA; and
e) sequencing tagged DNA, the amplified DNA or the isolated DNA.

In a further aspect, the invention provides a method for genome sequence and/or epigenome analysis comprising the steps:
a) providing a sample comprising genomic DNA;
b) adding at least one engineered transposome complex according to the invention;
c) optionally amplifying tagged DNA;
d) optionally isolating the amplified DNA; and
e) sequencing tagged DNA, the amplified DNA or the isolated DNA.

In some embodiments, the sample further comprises RNA.

In some embodiments, the methods further comprise the steps of tagging the RNA, optionally amplifying the tagged RNA, optionally isolating the amplified cDNA and optionally sequencing the tagged RNA, amplified cDNA or isolated cDNA. Suitably, the RNA is tagged using a polyA capture probe(s) which may comprising an RNA tagging sequence.

In a further aspect, the invention provides a method for making a DNA sequence library or libraries and an RNA sequence library or libraries comprising the steps:
a) providing a sample comprising genomic DNA and RNA;
b) (i) adding at least one engineered transposome complex; and
   (ii) tagging the RNA;
c) optionally amplifying tagged DNA and/or tagged RNA;
d) optionally isolating the amplified DNA and/or the amplified cDNA; and
e) optionally sequencing tagged DNA, the amplified DNA or the isolated DNA and/or optionally sequencing tagged RNA, the amplified cDNA or the isolated cDNA.

In a further aspect, the invention provides a method for DNA sequencing and RNA sequencing comprising the steps:
a) providing a sample comprising genomic DNA and RNA;
b) (i) adding at least one engineered transposome complex; and
   (ii) tagging the RNA;
c) optionally amplifying tagged DNA and/or tagged RNA;
d) optionally isolating the amplified DNA and/or the amplified cDNA; and
e) sequencing tagged DNA, the amplified DNA or the isolated DNA and sequencing tagged RNA, the amplified cDNA or the isolated cDNA.

In a further aspect, the invention provides a method for a method for genome sequence, epigenome and/or transcriptome analysis comprising the steps:
a) providing a sample comprising genomic DNA and RNA;
b) (i) adding at least one engineered transposome complex; and
   (ii) tagging the RNA;
c) optionally amplifying tagged DNA and/or tagged RNA;
d) optionally isolating the amplified DNA and/or the amplified cDNA; and
e) sequencing tagged DNA, the amplified DNA or the isolated DNA and sequencing tagged RNA, the amplified cDNA or the isolated cDNA.

In some embodiments, the sequencing comprises single-cell sequence analysis. Suitably, the method may use a microfluidic device. Suitably, the method may use a droplet-based microfluidic device and/or beads comprising an RNA tagging sequence(s).

In some embodiments, the engineered transposome complex comprises an oligonucleotide and an engineered transposase.

In some embodiments, the oligonucleotide comprises a sequencing primer site, a tagging sequence and/or a mosaic end.

In some embodiments, the oligonucleotide comprises a 5′ phosphate group.

In some embodiments, the engineered transposase comprises a transposase operably linked to a polypeptide that binds to a component of heterochromatin and/or euchromatin. In preferred embodiments, the engineered transposase comprises a transposase operably linked to a polypeptide that binds to a component of heterochromatin.

In some embodiments, the polypeptide binds to methylated histone.

In some embodiments, the polypeptide binds to H3K9me3, H3K27me3 and/or H3K36me3.

In some embodiments, the polypeptide binds to H3K9me3.

In some embodiments, the polypeptide comprises a chromodomain, a bromodomain, a HMG-box domain, a JmJc domain, a KRAB domain or a PWWP domain.

In some embodiments, the polypeptide comprises a chromodomain.

In some embodiments, the chromodomain is selected from the chromodomain of heterochromatin protein 1-α, of chromobox protein homolog 2, of chromobox protein homolog 5, of chromobox protein homolog 7, of chromobox protein homolog 8, of yeast protein Eaf3 or of M phase phosphoprotein 8.

In preferred embodiments, the chromodomain is the chromodomain of heterochromatin protein 1-α.

In some embodiments, the transposase is a DD [E/D] transposase.

In some embodiments, the transposase is selected from Tn5, Sleeping Beauty, Tn10, Drosophila P element, bacteriophage Mu, Tc1/Mariner, IS10 and IS50.

In preferred embodiments, the transposase is Tn5.

In preferred embodiments, the engineered transposase comprises Tn5 operably linked to a chromodomain, preferably chromodomain of heterochromatin protein 1-α.

In some embodiments, the engineered transposase comprises:
a) a sequence having at least 70% sequence identity to the sequence set forth in SEQ ID NO: 9; and/or
b) a sequence having at least 70% sequence identity to the sequence set forth in SEQ ID NO: 22 or SEQ ID NO: 24.

In some embodiments, the engineered transposase comprises a sequence having at least 70% sequence identity to the sequence set forth in SEQ ID NO: 1, SEQ ID NO: 3, SEQ ID NO: 5 or SEQ ID NO: 7.

In preferred embodiments, the engineered transposase comprises a sequence having at least 70% sequence identity to the sequence set forth in SEQ ID NO: 1. In some embodiments, the engineered transposase comprises a sequence having at least 70% sequence identity to the sequence set forth in SEQ ID NO: 3. In some embodiments, the engineered transposase comprises a sequence having at least 70% sequence identity to the sequence set forth in SEQ ID NO: 5. In some embodiments, the engineered transposase comprises a sequence having at least 70% sequence identity to the sequence set forth in SEQ ID NO: 7.

In some embodiments, the analysis determines genomic copy number variants (CNVs). In some embodiments, the analysis determines single nucleotide variations (SNV), for example within single cells.

In some embodiments, step b) further comprises adding at least one further transposome complex.

In some embodiments:
a) the at least one engineered transposome complex; and
b) the at least one further transposome complex,
   each binds to a different methylated histone.

In some embodiments:
a) the at least one engineered transposome complex; and
b) the at least one further transposome complex,
   each preferentially binds to a different methylated histone.

In some embodiments:
a) the at least one engineered transposome complex; and
b) the at least one further transposome complex,
   each has a different methylated histone binding specificity.

In some embodiments, the tagging sequence of the at least one engineered transposome complex differs from the tagging sequence of the at least one further transposome complex.

In some embodiments, the sample comprising genomic DNA is a sample of isolated cells, tissue, or whole organs. In some embodiments, the sample has not been pre-processed. In some embodiments, the sample comprising genomic DNA comprises genomic DNA which has been extracted from isolated cells, tissue, or whole organs, and optionally fragmented.

In some embodiments, nuclei in the sample have been permeabilized.

In some embodiments, the sample comprising genomic DNA is a sample comprising permeabilized nuclei.

In some embodiments, the sample comprising genomic DNA is a sample comprising permeabilized cells.

In some embodiments, the sample comprising genomic DNA comprises a single cell. In some embodiments, the sample comprising genomic DNA comprises an intact single cell.

In some embodiments, the sequencing comprises single-cell sequence analysis.

In some embodiments, the signals obtained from the at least one further transposome complex and the at least one engineered transposome complex at a DNA locus are compared.

In some embodiments, the at least one further transposase and/or at least one further transposome complex binds to euchromatin.

In some embodiments, the ratio between signals obtained from the at least one further transposome complex and the at least one engineered transposome complex at a DNA locus is determined. In some embodiments, an increase in the ratio indicates an increase in open chromatin. In some embodiments, a decrease in the ratio indicates an increase in compact chromatin.

In a further aspect, the invention provides an engineered transposase as described herein.

In a further aspect, the invention provides an engineered transposase comprising a transposase operably linked to a polypeptide that binds to a component of heterochromatin and/or euchromatin.

In a further aspect, the invention provides an engineered transposase comprising a transposase operably linked to a polypeptide that binds to a component of heterochromatin.

In some embodiments, the polypeptide binds to methylated histone.

In some embodiments, the polypeptide binds to H3K9me3, H3K27me3 and/or H3K36me3.

In some embodiments, the polypeptide binds to H3K9me3.

In some embodiments, the polypeptide comprises a chromodomain, a bromodomain, a HMG-box domain, a JmJc domain, a KRAB domain or a PWWP domain.

In some embodiments, the polypeptide comprises a chromodomain.

In some embodiments, the chromodomain is selected from the chromodomain of heterochromatin protein 1-α, of chromobox protein homolog 2, of chromobox protein homolog 5, of chromobox protein homolog 7, of chromobox protein homolog 8, of yeast protein Eaf3 or of M phase phosphoprotein 8.

In preferred embodiments, the chromodomain is the chromodomain of heterochromatin protein 1-α.

In some embodiments, the transposase is selected from Tn5, Sleeping Beauty, Tn10, Drosophila P element, bacteriophage Mu, Tc1/Mariner, IS10 and IS50.

In preferred embodiments, the transposase is Tn5.

In preferred embodiments, the engineered transposase comprises Tn5 operably linked to a chromodomain, preferably chromodomain of heterochromatin protein 1-α.

In some embodiments, the engineered transposase comprises:
a) a sequence having at least 70% sequence identity to the sequence set forth in SEQ ID NO: 9; and/or
b) a sequence having at least 70% sequence identity to the sequence set forth in SEQ ID NO: 22 or SEQ ID NO: 24.

In some embodiments, the engineered transposase comprises a sequence having at least 70% sequence identity to the sequence set forth in SEQ ID NO: 1, SEQ ID NO: 3, SEQ ID NO: 5 or SEQ ID NO: 7.

In preferred embodiments, the engineered transposase comprises a sequence having at least 70% sequence identity to the sequence set forth in SEQ ID NO: 1. In some embodiments, the engineered transposase comprises a sequence having at least 70% sequence identity to the sequence set forth in SEQ ID NO: 3. In some embodiments, the engineered transposase comprises a sequence having at least 70% sequence identity to the sequence set forth in SEQ ID NO: 5. In some embodiments, the engineered transposase comprises a sequence having at least 70% sequence identity to the sequence set forth in SEQ ID NO: 7.

In a further aspect, the invention provides an engineered transposome complex as described herein.

In a further aspect, the invention provides an engineered transposome complex comprising an oligonucleotide and an engineered transposase according to the invention.

In some embodiments, the oligonucleotide comprises a sequencing primer site, a tagging sequence and/or a mosaic end. In some embodiments, the oligonucleotide comprises a sequencing primer site, a tagging sequence and a mosaic end.

In a further aspect, the invention provides a kit comprising:
a) at least one engineered transposase according to the invention and at least one further transposase; or
b) at least one engineered transposome complex according to the invention and at least one further transposome complex.

In a further aspect, the invention provides the use of an engineered transposase according to the invention for making a DNA sequence library or libraries.

In a further aspect, the invention provides the use of an engineered transposome according to the invention for making a DNA sequence library or libraries.

In a further aspect, the invention provides the use of an engineered transposase according to the invention for DNA sequencing.

In a further aspect, the invention provides the use of an engineered transposome according to the invention for DNA sequencing.

In a further aspect, the invention provides the use of an engineered transposase according to the invention for genome and epigenetic sequencing.

In a further aspect, the invention provides the use of an engineered transposome according to the invention for genome and epigenetic sequencing.

In a further aspect, the invention provides a method for making a DNA sequence library or libraries comprising the steps:
a) providing a sample comprising genomic DNA;
b) adding at least one engineered transposome complex comprising an oligonucleotide and an engineered transposase;
c) optionally amplifying tagged DNA; and
d) optionally isolating the amplified DNA,

wherein the engineered transposase comprises a transposase operably linked to a polypeptide that binds to a component of heterochromatin and/or euchromatin, preferably heterochromatin.

In a further aspect, the invention provides a method for DNA sequencing comprising the steps:
a) providing a sample comprising genomic DNA;
b) adding at least one engineered transposome complex comprising an oligonucleotide and an engineered transposase;
c) amplifying tagged DNA;
d) optionally isolating the amplified DNA; and
e) sequencing the amplified DNA or the isolated DNA,

wherein the engineered transposase comprises a transposase operably linked to a polypeptide that binds to a component of heterochromatin and/or euchromatin, preferably heterochromatin.

In a further aspect, the invention provides a method for genome sequence and/or epigenome analysis comprising the steps:
a) providing a sample comprising genomic DNA;
b) adding at least one engineered transposome complex comprising an oligonucleotide and an engineered transposase;
c) amplifying tagged DNA;
d) optionally isolating the amplified DNA; and
e) sequencing the amplified DNA or the isolated DNA,

wherein the engineered transposase comprises a transposase operably linked to a polypeptide that binds to a component of heterochromatin and/or euchromatin, preferably heterochromatin.

In a further aspect, the invention provides a method for making a DNA sequence library or libraries and an RNA sequence library or libraries comprising the steps:
a) providing a sample comprising genomic DNA and RNA;
b) (i) adding at least one engineered transposome complex comprising an oligonucleotide and an engineered transposase; and
   (ii) tagging the RNA;
c) optionally amplifying tagged DNA and/or tagged RNA; and
d) optionally isolating the amplified DNA and/or the amplified cDNA,

wherein the engineered transposase comprises a transposase operably linked to a polypeptide that binds to a component of heterochromatin and/or euchromatin, preferably heterochromatin.

In a further aspect, the invention provides a method for DNA sequencing and RNA sequencing comprising the steps:
a) providing a sample comprising genomic DNA and RNA;
b) (i) adding at least one engineered transposome complex comprising an oligonucleotide and an engineered transposase; and
   (ii) tagging the RNA;
c) amplifying tagged DNA and tagged RNA;
d) optionally isolating the amplified DNA and/or the amplified cDNA; and
e) sequencing the amplified DNA or the isolated DNA and sequencing the amplified cDNA or the isolated cDNA,

wherein the engineered transposase comprises a transposase operably linked to a polypeptide that binds to a component of heterochromatin and/or euchromatin, preferably heterochromatin.

In a further aspect, the invention provides a method for genome sequence, epigenome and/or transcriptome analysis comprising the steps:
a) providing a sample comprising genomic DNA and RNA;
b) (i) adding at least one engineered transposome complex comprising an oligonucleotide and an engineered transposase; and
   (ii) tagging the RNA;
c) amplifying tagged DNA and tagged RNA;
d) optionally isolating the amplified DNA and/or the amplified cDNA; and
e) sequencing the amplified DNA or the isolated DNA and sequencing the amplified cDNA or the isolated cDNA,

wherein the engineered transposase comprises a transposase operably linked to a polypeptide that binds to a component of heterochromatin and/or euchromatin, preferably heterochromatin.

In a further aspect, the invention provides a method for making a DNA sequence library or libraries comprising the steps:
a) providing a sample comprising genomic DNA;
b) adding at least one engineered transposome complex and at least one further transposome complex;
c) optionally amplifying tagged DNA; and
d) optionally isolating the amplified DNA,

wherein the at least one engineered transposome complex comprises an oligonucleotide and an engineered transposase, and wherein the engineered transposase comprises a transposase operably linked to a polypeptide that binds to a component of heterochromatin and/or euchromatin, preferably heterochromatin.

In a further aspect, the invention provides a method for DNA sequencing comprising the steps:
a) providing a sample comprising genomic DNA;
b) adding at least one engineered transposome complex and at least one further transposome complex;
c) amplifying tagged DNA;
d) optionally isolating the amplified DNA; and
e) sequencing the amplified DNA or the isolated DNA.

wherein the at least one engineered transposome complex comprises an oligonucleotide and an engineered transposase, and wherein the engineered transposase comprises a transposase operably linked to a polypeptide that binds to a component of heterochromatin and/or euchromatin, preferably heterochromatin.

In a further aspect, the invention provides a method for genome sequence and/or epigenome analysis comprising the steps:
a) providing a sample comprising genomic DNA;
b) adding at least one engineered transposome complex and at least one further transposome complex;
c) amplifying tagged DNA;
d) optionally isolating the amplified DNA; and
e) sequencing the amplified DNA or the isolated DNA.

wherein the at least one engineered transposome complex comprises an oligonucleotide and an engineered transposase, and wherein the engineered transposase comprises a transposase operably linked to a polypeptide that binds to a component of heterochromatin and/or euchromatin, preferably heterochromatin.

In a further aspect, the invention provides a method for making a DNA sequence library or libraries and an RNA sequence library or libraries comprising the steps:
a) providing a sample comprising genomic DNA and RNA;
b) (i) adding at least one engineered transposome complex and at least one further transposome complex; and
   (ii) tagging the RNA;
c) optionally amplifying tagged DNA and/or tagged RNA; and
d) optionally isolating the amplified DNA and/or the amplified cDNA,

wherein the at least one engineered transposome complex comprises an oligonucleotide and an engineered transposase, and wherein the engineered transposase comprises a transposase operably linked to a polypeptide that binds to a component of heterochromatin and/or euchromatin, preferably heterochromatin.

In a further aspect, the invention provides a method for DNA sequencing and RNA sequencing comprising the steps:
a) providing a sample comprising genomic DNA and RNA;
b) (i) adding at least one engineered transposome complex and at least one further transposome complex; and
   (ii) tagging the RNA;
c) amplifying tagged DNA and tagged RNA;
d) optionally isolating the amplified DNA and/or the amplified cDNA; and
e) sequencing the amplified DNA or the isolated DNA and sequencing the amplified cDNA or the isolated cDNA,

wherein the at least one engineered transposome complex comprises an oligonucleotide and an engineered transposase, and wherein the engineered transposase comprises a transposase operably linked to a polypeptide that binds to a component of heterochromatin and/or euchromatin, preferably heterochromatin.

In a further aspect, the invention provides a method for genome sequence, epigenome and/or transcriptome analysis comprising the steps:
a) providing a sample comprising genomic DNA and RNA;
b) (i) adding at least one engineered transposome complex and at least one further transposome complex; and
   (ii) tagging the RNA;
c) amplifying tagged DNA and tagged RNA;
d) optionally isolating the amplified DNA and/or the amplified cDNA; and
e) sequencing the amplified DNA or the isolated DNA and sequencing the amplified cDNA or the isolated cDNA,

wherein the at least one engineered transposome complex comprises an oligonucleotide and an engineered transposase, and wherein the engineered transposase comprises a transposase operably linked to a polypeptide that binds to a component of heterochromatin and/or euchromatin, preferably heterochromatin.

### BRIEF DESCRIPTION OF THE FIGURES

FIG. 1: Tn5 transposase is able to tagment compacted chromatin featuring H3K9me3. a, General scheme of TAM-ChIP technique (created with BioRender.com). A primary antibody (ChIP-validated antibody, dark grey) binds to a specific histone modification (light grey) over the genome (blue-red). A secondary antibody (TAM-ChIP conjugate, blue) is linked to the Tn5 transposon, which is made of Tn5 transposase (yellow) and the respective barcoded adapters (green). Upon the binding of the secondary antibody to the primary antibody, the linked Tn5 transposase targets and cuts the genomic regions flanking the histone modification, adding the barcoded adapters. TAM-ChIP was performed on two biological replicates for each condition (H3K4me3, H3K9me3 and NoAb). b, H3K4me3 (green) and H3K9me3 (red) enrichment profiles obtained either by ChIP-seq or TAM-ChIP-seq, compared with Input ChIP control (violet). c, Hilbert curves representing overlap of signals obtained by H3K4me3 (green) and H3K9me3 (red) obtained by ChIP-seq with H3K4me3 and H3K9me3 (blue) obtained by TAM-ChIP-seq. Data for chromosome 19 are presented. d, Enrichment profile of heterochromatic genes FAM5B, NTF3, CACNA1E obtained from TAM-ChIP libraries assessed by Real Time-qPCR confirms Tn5 is able to target heterochromatic loci when redirected by H3K9me3 antibody. For each biological replicate three technical replicates were analyzed by Real-Time qPCR; one of the two H3K4me3 biological replicate was excluded because no significant signal was detected for any condition. Bars represent standard deviations (n=3 technical replicates). Data shown in b, c and d refer to experiments performed on Caki-1 cell line.

FIG. 2: Hybrid CD (HP1α)-Tn5 targets H3K9me3 chromatin regions. a, According to the cloning strategy, two CD (HP1α)-containing regions (spanning amino acids 1-93 and 1-112) were linked to Tn5, using either a 3 or 5 poly-tyrosine-glycine-serine (TGS) linker, resulting in four hybrid constructs: TnH #1-4 (TnH #1: 93aaCD (HP1α)-3×(TGS)-Tn5; TnH #2: 93aaCD (HP1α)-5×(TGS)-Tn5; TnH #3: 112aaCD (HP1α)-3×(TGS)-Tn5; TnH #4: 112aaCD (HP1α)-5×(TGS)-Tn5 (created with BioRender.com). b-c, Tagmentation profiles relative to the four hybrid constructs (TnH #1-4) showing no difference in tagmentation efficiency relative to the native Tn5 enzyme (Nextera and Tn5 in-house produced) when targeting either genomic DNA, panel b, or native chromatin on permeabilized nuclei, panel c. d, Enrichment profiles relative to ATAC-seq performed with the four hybrid constructs (TnH #1-4, red) compared with native Tn5 enzyme (Nextera and Tn5 in-house produced) and with H3K4me3 and H3K9me3 ChIP-seq signals (green). e, Distribution of the enrichment of four TnH hybrid constructs (TnH #1-4) relative to genomic background in regions enriched for H3K4me3 (orange) or H3K9me3 (blue) expressed as log 2 (ratio) of the signal over the genomic Input. Enrichment over the same regions for native Tn5 enzyme (Nextera and Tn5 in-house produced), H3K4me3 and H3K9me3 ChIP-seq are reported as reference. Ec: global enrichment over H3K9me3-marked regions; Eo: global enrichment over H3K4me3-marked regions; Mc: modal enrichment over H3K9me3-marked regions; Mo: modal enrichment over H3K4me3-marked regions. Data shown in b, c and d refer to experiments performed on Caki-1 cell line.

FIG. 3: Tn5 transposon is able to target H3K9me3-enriched regions. a, Enrichment profile of H3K4me3 (green) and H3K9me3 (red)-associated regions obtained by ChIP-seq compared to Tn5 (green) and TnH (red) tagmentation profile obtained by ATAC-seq. ChIP-seq input track is shown as control (violet). b, Distribution of the enrichment of Tn5 and TnH transposons relative to genomic background in regions enriched for H3K4me3 (orange) or H3K9me3 (blue) expressed as log 2 (ratio) of the signal over the genomic Input. Enrichment over the same regions for H3K4me3 and H3K9me3 ChIP-seq are reported as reference. Ec: global enrichment over H3K9me3-marked regions; Eo: global enrichment over H3K4me3-marked regions; Mc: modal enrichment over H3K9me3-marked regions; Mo: modal enrichment over H3K4me3-marked regions. c, General scheme of the GET-seq transposon structure. Standard Tn5ME-A oligo was replaced by 49 nt oligos composed by 22 nt for Read 1 sequencing primer binding, 8 nt tags to discriminate Tn5 from TnH tagmentation products, and standard 19-bp ME sequence for transposase binding (created with BioRender.com). d, Hilbert curves representing the overlap of signal obtained by Tn5 or TnH (red) with H3K4me3 (blue) and H3K9me3 (green). Data for chromosome 19 are presented. Data shown in a,b and d refers to experiments performed on Caki-1 cells.

FIG. 4: Optimization of ATAC-seq protocol introducing a combination of Tn5 and TnH transposases. a, Effect of altering Tn5 (green) to TnH (red) ratio on tagmentation profiles when adding both enzymes simultaneously at the beginning of the 60 minutes of the transposition reaction. b, Sequential addition of the same quantity of Tn5 and then TnH enzyme after 30 minutes of the transposition reaction results in a balanced distribution of enrichment signals between the two enzymes. Experiments performed on Caki-1 cell line.

FIG. 5: Assessment of scGET-seq strategy and genomic copy number at the single-cell level. a, Abundance of unique cell barcodes retrieved by scATAC-seq performed on Caki-1 cells using the standard the provided ATAC transposition enzyme (10× Tn5; 10× Genomics) (blue) compared to cell barcodes countable by TnH (orange) or Tn5 (green) alone. scGET-seq performance (Tn5+TnH) is represented in red. The curves are largely overlapping, indicating no evident bias in single cell identification. b, Distribution of per-cell coverage is reported for 10× Tn5 (blue) and for signal obtained by TnH (orange) and Tn5 (green). While Tn5 is comparable to 10× Tn5, TnH returns higher coverages. c, UMAP embedding showing individual cells in a mixture of Caki-1/HeLa at known proportions (80:20). Cells are identified according to a signature calculated on specific DHS identified from bulk studies. d, Spearman's correlation between the segmentation profile of Caki-1 and Hela cells at increasing resolution. Signal from bulk sequencing is compared to average cell signal obtained in single cell profiling. scGET-seq (orange) shows consistently higher correlation compared to standard scATACseq (blue). e, Segmentation profiles in individual cells profiled by 10× Tn5 (scATAC-seq) (upper panel) or TnH scGET-seq (lower panel) at 500 kb. On top of each heatmap the genome-wide coverage of bulk sequencing of corresponding cell lines is represented. Centromeric regions and gaps (in white) have been excluded from the analysis. f, Spearman's correlation between the segmentation profiles and the density of regulatory elements in the GeneHancer catalog. g, Comparison between Tn5/TnH bulk and pseudo-bulk dataset. Tn5 (green) and TnH (red) enrichment profiles obtained from scGET-seq (pseudo-bulk) or from ATAC-seq performed by using the two enzymes separately, compared with H3K4me3 (green) and H3K9me3 (red) ChIP-seq data. Data shown refer to experiments performed on Caki-1 cells. h, UMAP embedding showing individual cells in a mixture of Caki-1/HeLa at known proportions (80:20) profiled by standard scATAC-seq. Cells are identified according to a signature calculated on specific DHS identified from bulk studies i, Heatmap showing the performance of two different classifiers on genomic alterations (amplifications, deletions and normal segments) in HeLa and CaKi-1 cells. Each classifier has been trained at increasing resolution on scGET-seq and scATAC-seq data separately. Both classifiers perform worse on Hela cells than in CaKi-1 cells given the lower numerosity.

FIG. 6: Copy Number analysis at multiple resolutions. a, Segmentation profiles in individual cells profiled by 10× Tn5 (scATAC-seq) (upper panel) or TnH scGET-seq (lower panel) at 1 Mb. b, Segmentation profiles in individual cells profiled by 10× Tn5 (scATAC-seq) (upper panel) or TnH scGET-seq (lower panel) at 10 Mb. On top of each heatmap the genome-wide coverage of bulk sequencing of corresponding cell lines is represented. Centromeric regions and gaps (in white) have been excluded from the analysis.

FIG. 7: scGET-seq analysis on PDX samples, a, UMAP embedding of individual cells as in FIG. 14, colored by the time PDX were harvested. b, Segmentation profiles in individual cells profiled by scGET-seq at 1 Mb resolution expressed as log 2 (ratio) over the median signal. Cells are clustered according to genetic clones. Red: positive values; Blue: negative values. Centromeric regions (white) have been excluded from the analysis because they correspond to low mapping and not fully characterized regions.

FIG. 8: scGET-seq analysis on PDX samples, a-b, UMAP embeddings of scGET-seq profiles. Cells are colored according to the clones derived from segmentation data, panel a, or epigenome analysis, panel b. c, Abundance of genetic clones over time; colors match the UMAP in panel a. d, Abundance of epigenetic clones over time; colors match the UMAP in panel b. e, Dot plot representing functional enrichment of genes associated to DHS regions enriched in clone 1 and 2. Dot size is proportional to the number of genes, dot color encodes for Enrichr combined score, position on the x-axis is the −10 log10 (p-value) of the term enrichment. Terms are recovered from KEGG pathways (K) or Reactome pathways (R). f, Scatterplot of allele frequency of somatic mutations identified by Exome sequencing of the primary tumor in relation to the allele frequency detected by genotyping scGET-seq data. Dot size is proportional to coverage in scGETseq, while color matches the clones in panels a and c; Grey dots are mutations shared by two clones. Pearson correlation coefficient and the associated p-value are reported (n=389). g, Representative mutations of COSMIC Hallmark genes found in scGET-seq data which were not present in the primary tumor. Each mutation is associated to the corresponding genetic clone using the appropriate color code.

FIG. 9: scGET-seq profiling of NIH-3T3 cells knocked-down for Kdm5c. a, UMAP embedding showing the location of cells transfected with shKdm5c or shScr. b, UMAP embedding of individual cells coloured by the read coverage. Two main clusters appear depending on the coverage. c-d, UMAP embedding highlighting the density of cells with high signal over pericentromeric heterochromatin marked by the major primer (see text), as recovered by TnH, panel c, or Tn5, panel d. The two signals are unevenly distributed and tend to localize where higher amounts of shScr cells are. All these data refer to experiments performed on NIH-3T3 cell line.

FIG. 10: scGET-seq profiling of NIH-3T3 cells knocked-down for Kdm5c. a, Distribution of early-to-late ratio of 2-stage Repli-seq data for NIH-3T3 cells. Violin plots represent the value of log 2 (E/L) values over DHS regions which are differential in the high-vs-low coverage cells in FIG. 9a (Mann-Whitney U=36169.5, p=1.403e-84). b, Distribution of lamin-B1 DamID scores for NIH-3T3 cells. Violin plots represent the value of DamID scores over DHS regions which are differential in the high-vs-low coverage cells in FIG. 9a (Mann-Whitney U=723.0, p=4.621e-6). c, UMAP embedding of individual cells coloured by cell groups, identified by Leiden algorithm with resolution parameter set to 0.2. d, Results of the linear model calculating the group-wise differences between TnH and Tn5 enrichment. For each group we reported the coefficient of the model, the p-value and the Benjamini-Hochberg corrected p-value. Values are reported for the two genomic regions including the Major primers (see text). Barplot indicates the proportion of shScr-treated for each cell group.

FIG. 11: scGET-seq profiling of a developmental model of iPSC. a, Graph embedding of single cells coloured by cell type. b, Graph embedding of individual cells coloured by cell group as identified by Nested Stochastic Block Model. c, Same as in panel b, but cells are coloured by the donor. d, Graph embedding of scGET-seq profiled cells, coloured by differentiation potential, as result of Palantir algorithm. (FIB: Fibroblasts, iPSC: induced-Pluripotent Stem Cells, NPC: Neural Progenitor Cells).

FIG. 12: scGET-seq profiling of a developmental model of iPSC. a, Graph embedding of individual cells coloured by the density of cells having an undifferentiated score in the 3rd quartile of values. b, Proportion of cells derived from individual donors in each cell group identified by schist. c, Schematic representation of the phase portraits underlying Chromatin Velocity. In RNA-velocity, the time derivative of the unspliced/spliced RNA is used to estimate synthesis or degradation of RNA; in Chromatin Velocity, the same procedure is applied on Tn5/TnH data to estimate chromatin relaxation or compaction. d, Graph embedding of individual cells coloured by latent time, estimated using scvelo. e, Violin plot shows the distributions of latent times of donor-specific NPC (Mann-Whitney U=8871194, p=3.775e-111).

FIG. 13: Chromatin velocity. a-b, Graph embedding of differentiating single cell as in FIG. 11b, e. Cells are coloured by differentiation potential, panel a, or cell group, panel b. Arrows indicate the epigenetic velocity extracted using scvelo. Arrow length is proportional to the cell velocity. c, Heatmap representing the velocity over top 1,655 dynamic regions according to the model likelihood (rows). Regions are selected to be in the 95th percentile of the likelihood values. Columns are individual cells, sorted according to the latent time estimated by scvelo. The coloured bar on the top indicates cell groups as appear in panel b. d, Selected KEGG pathways enriched for genes associated to the top dynamic regions. Association was performed by closest gene. e, Schematic representation of the TF analysis. The matrix of velocities calculated over the top dynamic regions is multiplied by the matrix of Total Binding Affinity calculated for all PWM in HOCOMOCO v11 over the same regions. The final matrix contains a single value for each cell for each PWM representing the relevance of a specific TF in the dynamic process happening over that cell. f, PLS plot of cell TF analysis matrix. Each dot represents the centroid of all cells belonging to a specific cell group, dots are coloured according to cell groups in panel b. Arrows indicate the loading of the top 3 PWM on each axis. Heatmap surrounding the scatterplot indicates the average Differentiation Potential (DP) of individual cells over the y axis. g, Heatmap shows average expression profiles of TF with the top 5 most negative and top 5 most positive loading on PLS2 during the early brain development. Darker colour indicates higher expression. w.p.c.: weeks post conception.

FIG. 14: Analysis of Patient Derived Organoids by scGET-seq. a, evaluation of clonal structure of two PDO (CRC6 and CRC17) by exome sequencing; the histogram show the distribution of the cancer cell fraction estimated from the analysis of somatic mutations; in both organoids we observe a monoclonal structure; b, 5× (left panel) and 10× (right panel) magnification contrast phase images of PDO #CRC17 obtained from a liver metastasis of a CRC patient; c, genetic structure of CRC6 and CRC17 as revealed by scGET-seq (heatmap) and exome sequencing (panels above and below the heatmap). scGET-seq data are expressed as normalized log 2 (ratio) of the signal in 1 Mb windows with respect to the average per-cell coverage. Centromeric regions and genome gaps were excluded from the analysis and colored in white. d, distribution of the marginal posterior probability of the number of cell clusters identified using TnH-derived reads (orange) or Tn5-derived reads (blue). Analysis of clonal structure with Tn5-derived reads, as in scATAC-seq, may lead to overclustering. e, analysis of the performance of variant calling in PDO samples as a function of coverage on the profiled variants. The shaded interval represents the range of values for two samples, the solid line represents the geometric mean. Sensitivity is calculated as TP/(TP+FN), Precision is calculated as TP/(TP+FP), where TP=alleles correctly identified; FP=alleles identified by scGET-seq and not by Exome Sequencing; FN=alleles identified by Exome Sequencing and not by scGET-seq. Depth threshold is applied on variants profiled by scGET-seq.

FIG. 15: scGETseq defines cell identity and developmental trajectories of FIB, iPSC and NPC. a, UMAP embedding showing scGET-seq profiling of human fibroblasts (FIB), induced Pluripotent Stem Cells (iPSC) and Neural Precursor Cells (NPC). Black arrow shows a small subset of FIB and NPCs clustering alongside iPSC. b, UMAP embedding showing scRNA-seq profiling of the same cell populations derived from the same samples as in panel a. c, the profiles show the pseudobulk Tn5 signal for three selected regions among the top differentially enriched in the three cell types; tracks are colored according to cell types as in panels a and b; a UMAP embedding colored by the level of expression of the corresponding gene is reported on the right of each profile. d, UMAP embedding of cells profiled by scGET-seq and colored by entropy (differentiation potential) as estimated by Palantir. e, heatmap showing the enrichment of Tn5 over the top 20 regions associated with a high entropy as result of a Generalized Linear Model. The first annotation row is colored by cell cluster, the second annotation row is colored by the cell type. f, UMAP embedding of cells profiled by scRNA-seq and colored by the expression signature derived from genes associated to regions depicted in panel.

FIG. 16: scGET-seq profiling of a developmental model of iPSC. a, UMAP embedding of individual cells colored by the probability of being included in a trajectory branch estimated by Palantir. Three major branches have been identified, roughly corresponding to the three cell types profiled in this study. b, UMAP embedding of individual cells colored by cell clusters. c, Heatmap shows average expression profiles of TF with the top 10 most negative on PLS2 during the early brain development. Darker color indicates higher expression. w.p.c.: weeks post conception.

FIG. 17: Chromatin velocity. a, UMAP embedding of differentiating single cells profiled by scGET-seq. Cells are colored by velocity pseudotime, arrow streams indicate the Chromatin velocity extracted using scvelo b, UMAP embedding of differentiating single cells profiled by scRNA-seq. Cells are colored by velocity pseudotime, arrow streams indicate the RNA velocity extracted using scvelo. c, Selected terms enriched for genes associated to the top dynamic regions. d, Schematic representation of the TF analysis. The matrix of velocities calculated over the top dynamic regions is multiplied by the matrix of Total Binding Affinity calculated for all PWM in HOCOMOCO v11 over the same regions. The final matrix contains a single value for each cell for each PWM representing the relevance of a specific TF in the dynamic process happening over that cell. e, PLS plot of cell TF analysis matrix. Each dot represents the centroid of all cells belonging to a specific cell group, dots are colored according to cell groups in FIG. 16b. Arrows indicate the loading of the top 4 PWM in each quadrant. The colored contours indicate the density estimates of the three cell types. g, Heatmap shows average expression profiles of TF with the top 10 most negative on PLS1 during the early brain development. Darker color indicates higher expression. w.p.c.: weeks post conception.

FIG. 18: GET²-Seq-Library profiles obtained with GET²-seq using Caki-1 nuclei as input for the assay. a, GET²-seq library profiles obtained replacing 10× standard transposase in the Chromium Single Cell Multiome ATAC+Gene Expression kit (10× Genomics) reagent kit; b, library profile for RNA corresponding to the same cells analyzed in panel A.

### DETAILED DESCRIPTION OF THE INVENTION

### Engineered Transposase

In one aspect, the present invention provides an engineered transposase comprising a transposase operably linked to a polypeptide that binds to a component of chromatin.

The engineered transposase may have been redirected to bind to a different component of chromatin compared to the corresponding unmodified transposase. Alternatively, the engineered transposase may have been redirected to bind to an additional component of chromatin compared to the corresponding unmodified transposase. Thus, the tropism of the transposase may be modified, targeting it directly towards a different or an additional component of chromatin. By targeting directly, it is meant that the engineered transposase of the invention directly may bind to a component of chromatin without an antibody intermediate. Thus, the engineered transposase of the invention may retain the affinity of the corresponding unmodified transposase, e.g. the engineered transposase of the invention may bind to the same component of chromatin as the corresponding unmodified transposase and to an additional component of chromatin.

An illustrative example of an engineered transposase (TnH #3) amino acid sequence is shown as SEQ ID NO: 1.

SEQ ID NO: 1
MGKKTKRTADSSSSEDEEEYVVEKVLDRRVVKGQVEYLLKWKGFSEEHNT
WEPEKNLDCPELISEFMKKYKKMKEGENNKPREKSESNKRKSNFSNSADD
IKSKKKREQSNDTGSTGSTGSHMITSALHRAADWAKSVFSSAALGDPRRT
ARLVNVAAQLAKYSGKSITISSEGSKAMQEGAYRFIRNPNVSAEAIRKAG
AMQTVKLAQEFPELLAIEDTTSLSYRHQVAEELGKLGSIQDKSRGWWVHS
VLLLEATTFRTVGLLHQEWWMRPDDPADADEKESGKWLAAAATSRLRMGS
MMSNVIAVCDREADIHAYLQDKLAHNERFVVRSKHPRKDVESGLYLYDHL
KNQPELGGYQISIPQKGVVDKRGKRKNRPARKASLSLRSGRITLKQGNIT
LNAVLAEEINPPKGETPLKWLLLTSEPVESLAQALRVIDIYTHRWRIEEF
HKAWKTGAGAERQRMEEPDNLERMVSILSFVAVRLLQLRESFTPPQALRA
QGLLKEAEHVESQSAETVLTPDECQLLGYLDKGKRKRKEKAGSLQWAYMA
IARLGGFMDSKRTGIASWGALWEGWEALQSKLDGFLAAKDLMAQGIKIC

An illustrative example of a nucleic acid sequence encoding an engineered transposase (TnH #3) is shown as SEQ ID NO: 2.

SEQ ID NO: 2
ATGGGAAAGAAAACCAAGCGGACAGCTGACAGTTCTTCTTCAGAGGATGA
GGAGGAGTATGTTGTGGAGAAGGTGCTAGACAGGCGCGTGGTTAAGGGAC
AAGTGGAATATCTACTGAAGTGGAAAGGCTTTTCTGAGGAGCACAATACT
TGGGAACCTGAGAAAAACTTGGATTGCCCTGAGCTAATTTCTGAATTTAT
GAAAAAGTATAAGAAGATGAAGGAGGGTGAAAATAATAAACCCAGGGAGA
AGTCAGAAAGTAACAAGAGGAAATCCAATTTCTCAAACAGTGCCGATGAC
ATCAAATCTAAAAAAAAGAGAGAGCAGAGCAATGATACGGGGTCGACCGG
CTCAACTGGTTCCCATATGATTACCAGTGCACTGCATCGTGCGGCGGATT
GGGCGAAAAGCGTGTTTTCTAGTGCTGCGCTGGGTGATCCGCGTCGTACC
GCGCGTCTGGTGAATGTTGCGGCGCAACTGGCCAAATATAGCGGCAAAAG
CATTACCATTAGCAGCGAAGGCAGCAAAGCCATGCAGGAAGGCGCGTATC
GTTTTATTCGTAATCCGAACGTGAGCGCGGAAGCGATTCGTAAAGCGGGT
GCCATGCAGACCGTGAAACTGGCCCAGGAATTTCCGGAACTGCTGGCAAT
TGAAGATACCACCTCTCTGAGCTATCGTCATCAGGTGGCGGAAGAACTGG
GCAAACTGGGTAGCATTCAGGATAAAAGCCGTGGTTGGTGGGTGCATAGC
GTGCTGCTGCTGGAAGCGACCACCTTTCGTACCGTGGGCCTGCTGCATCA
AGAATGGTGGATGCGTCCGGATGATCCGGCGGATGCGGATGAAAAAGAAA
GCGGCAAATGGCTGGCCGCTGCTGCAACTTCGCGTCTGAGAATGGGCAGC
ATGATGAGCAACGTGATTGCGGTGTGCGATCGTGAAGCGGATATTCATGC
GTATCTGCAAGATAAACTGGCCCATAACGAACGTTTTGTGGTGCGTAGCA
AACATCCGCGTAAAGATGTGGAAAGCGGCCTGTATCTGTATGATCACCTG
AAAAACCAGCCGGAACTGGGCGGCTATCAGATTAGCATTCCGCAGAAAGG
CGTGGTGGATAAACGTGGCAAACGTAAAAACCGTCCGGCGCGTAAAGCGA
GCCTGAGCCTGCGTAGCGGCCGTATTACCCTGAAACAGGGCAACATTACC
CTGAACGCGGTGCTGGCCGAAGAAATTAATCCGCCGAAAGGCGAAACCCC
GCTGAAATGGCTGCTGCTGACCAGCGAGCCGGTGGAAAGTCTGGCCCAAG
CGCTGCGTGTGATTGATATTTATACCCATCGTTGGCGCATTGAAGAATTT
CACAAAGCGTGGAAAACGGGTGCGGGTGCGGAACGTCAGCGTATGGAAGA
ACCGGATAACCTGGAACGTATGGTGAGCATTCTGAGCTTTGTGGCGGTGC
GTCTGCTGCAACTGCGTGAATCTTTTACTCCGCCGCAAGCACTGCGTGCG
CAGGGCCTGCTGAAAGAAGCGGAACACGTTGAAAGCCAGAGCGCGGAAAC
CGTGCTGACCCCGGATGAATGCCAACTGCTGGGCTATCTGGATAAAGGCA
AACGCAAACGCAAAGAAAAAGCGGGCAGCCTGCAATGGGCGTATATGGCG
ATTGCGCGTCTGGGCGGCTTTATGGATAGCAAACGTACCGGCATTGCGAG
CTGGGGTGCGCTGTGGGAAGGTTGGGAAGCGCTGCAAAGCAAACTGGATG
GCTTTCTGGCCGCGAAAGACCTGATGGCGCAGGGCATTAAAATCTGC

A further illustrative example of an engineered transposase (TnH #1) amino acid sequence is shown as SEQ ID NO: 3.

SEQ ID NO: 3
MGKKTKRTADSSSSEDEEEYVVEKVLDRRVVKGQVEYLLKWKGFSEEHNT
WEPEKNLDCPELISEFMKKYKKMKEGENNKPREKSESNKRKSNTGSTGST
GSHMITSALHRAADWAKSVFSSAALGDPRRTARLVNVAAQLAKYSGKSIT
ISSEGSKAMQEGAYRFIRNPNVSAEAIRKAGAMQTVKLAQEFPELLAIED
TTSLSYRHQVAEELGKLGSIQDKSRGWWVHSVLLLEATTFRTVGLLHQEW
WMRPDDPADADEKESGKWLAAAATSRLRMGSMMSNVIAVCDREADIHAYL
QDKLAHNERFVVRSKHPRKDVESGLYLYDHLKNQPELGGYQISIPQKGVV
DKRGKRKNRPARKASLSLRSGRITLKQGNITLNAVLAEEINPPKGETPLK
WLLLTSEPVESLAQALRVIDIYTHRWRIEEFHKAWKTGAGAERQRMEEPD
NLERMVSILSEVAVRLLQLRESFTPPQALRAQGLLKEAEHVESQSAETVL
TPDECQLLGYLDKGKRKRKEKAGSLQWAYMAIARLGGFMDSKRTGIASWG
ALWEGWEALQSKLDGFLAAKDLMAQGIKIC

A further illustrative example of a nucleic acid sequence encoding an engineered transposase (TnH #1) is shown as SEQ ID NO: 4.

SEQ ID NO: 4
ATGGGAAAGAAAACCAAGCGGACAGCTGACAGTTCTTCTTCAGAGGATGA
GGAGGAGTATGTTGTGGAGAAGGTGCTAGACAGGCGCGTGGTTAAGGGAC
AAGTGGAATATCTACTGAAGTGGAAAGGCTTTTCTGAGGAGCACAATACT
TGGGAACCTGAGAAAAACTTGGATTGCCCTGAGCTAATTTCTGAATTTAT
GAAAAAGTATAAGAAGATGAAGGAGGGTGAAAATAATAAACCCAGGGAGA
AGTCAGAAAGTAACAAGAGGAAATCCAATACGGGGTCGACCGGCTCAACT
GGTTCCCATATGATTACCAGTGCACTGCATCGTGCGGCGGATTGGGCGAA
AAGCGTGTTTTCTAGTGCTGCGCTGGGTGATCCGCGTCGTACCGCGCGTC
TGGTGAATGTTGCGGCGCAACTGGCCAAATATAGCGGCAAAAGCATTACC
ATTAGCAGCGAAGGCAGCAAAGCCATGCAGGAAGGCGCGTATCGTTTTAT
TCGTAATCCGAACGTGAGCGCGGAAGCGATTCGTAAAGCGGGTGCCATGC
AGACCGTGAAACTGGCCCAGGAATTTCCGGAACTGCTGGCAATTGAAGAT
ACCACCTCTCTGAGCTATCGTCATCAGGTGGCGGAAGAACTGGGCAAACT
GGGTAGCATTCAGGATAAAAGCCGTGGTTGGTGGGTGCATAGCGTGCTGC
TGCTGGAAGCGACCACCTTTCGTACCGTGGGCCTGCTGCATCAAGAATGG
TGGATGCGTCCGGATGATCCGGCGGATGCGGATGAAAAAGAAAGCGGCAA
ATGGCTGGCCGCTGCTGCAACTTCGCGTCTGAGAATGGGCAGCATGATGA
GCAACGTGATTGCGGTGTGCGATCGTGAAGCGGATATTCATGCGTATCTG
CAAGATAAACTGGCCCATAACGAACGTTTTGTGGTGCGTAGCAAACATCC
GCGTAAAGATGTGGAAAGCGGCCTGTATCTGTATGATCACCTGAAAAACC
AGCCGGAACTGGGCGGCTATCAGATTAGCATTCCGCAGAAAGGCGTGGTG
GATAAACGTGGCAAACGTAAAAACCGTCCGGCGCGTAAAGCGAGCCTGAG
CCTGCGTAGCGGCCGTATTACCCTGAAACAGGGCAACATTACCCTGAACG
CGGTGCTGGCCGAAGAAATTAATCCGCCGAAAGGCGAAACCCCGCTGAAA
TGGCTGCTGCTGACCAGCGAGCCGGTGGAAAGTCTGGCCCAAGCGCTGCG
TGTGATTGATATTTATACCCATCGTTGGCGCATTGAAGAATTTCACAAAG
CGTGGAAAACGGGTGCGGGTGCGGAACGTCAGCGTATGGAAGAACCGGAT
AACCTGGAACGTATGGTGAGCATTCTGAGCTTTGTGGCGGTGCGTCTGCT
GCAACTGCGTGAATCTTTTACTCCGCCGCAAGCACTGCGTGCGCAGGGCC
TGCTGAAAGAAGCGGAACACGTTGAAAGCCAGAGCGCGGAAACCGTGCTG
ACCCCGGATGAATGCCAACTGCTGGGCTATCTGGATAAAGGCAAACGCAA
ACGCAAAGAAAAAGCGGGCAGCCTGCAATGGGCGTATATGGCGATTGCGC
GTCTGGGCGGCTTTATGGATAGCAAACGTACCGGCATTGCGAGCTGGGGT
GCGCTGTGGGAAGGTTGGGAAGCGCTGCAAAGCAAACTGGATGGCTTTCT
GGCCGCGAAAGACCTGATGGCGCAGGGCATTAAAATCTGC

A further illustrative example of an engineered transposase (TnH #2) amino acid sequence is shown as SEQ ID NO: 5.

SEQ ID NO: 5
MGKKTKRTADSSSSEDEEEYVVEKVLDRRVVKGQVEYLLKWKGFSEEHNT
WEPEKNLDCPELISEFMKKYKKMKEGENNKPREKSESNKRKSNTGSTGST
GSTGSTGSHMITSALHRAADWAKSVFSSAALGDPRRTARLVNVAAQLAKY
SGKSITISSEGSKAMQEGAYRFIRNPNVSAEAIRKAGAMQTVKLAQEFPE
LLAIEDTTSLSYRHQVAEELGKLGSIQDKSRGWWVHSVLLLEATTFRTVG
LLHQEWWMRPDDPADADEKESGKWLAAAATSRLRMGSMMSNVIAVCDREA
DIHAYLQDKLAHNERFVVRSKHPRKDVESGLYLYDHLKNQPELGGYQISI
PQKGVVDKRGKRKNRPARKASLSLRSGRITLKQGNITLNAVLAEEINPPK
GETPLKWLLLTSEPVESLAQALRVIDIYTHRWRIEEFHKAWKTGAGAERQ
RMEEPDNLERMVSILSEVAVRLLQLRESFTPPQALRAQGLLKEAEHVESQ
SAETVLTPDECQLLGYLDKGKRKRKEKAGSLQWAYMAIARLGGFMDSKRT
GIASWGALWEGWEALQSKLDGFLAAKDLMAQGIKIC

A further illustrative example of a nucleic acid sequence encoding an engineered transposase (TnH #2) is shown as SEQ ID NO: 6.

SEQ ID NO: 6
ATGGGAAAGAAAACCAAGCGGACAGCTGACAGTTCTTCTTCAGAGGATGA
GGAGGAGTATGTTGTGGAGAAGGTGCTAGACAGGCGCGTGGTTAAGGGAC
AAGTGGAATATCTACTGAAGTGGAAAGGCTTTTCTGAGGAGCACAATACT
TGGGAACCTGAGAAAAACTTGGATTGCCCTGAGCTAATTTCTGAATTTAT
GAAAAAGTATAAGAAGATGAAGGAGGGTGAAAATAATAAACCCAGGGAGA
AGTCAGAAAGTAACAAGAGGAAATCCAATACGGGGTCGACCGGCTCAACT
GGTTCCACCGGGTCCACGGGCTCGCATATGATTACCAGTGCACTGCATCG
TGCGGCGGATTGGGCGAAAAGCGTGTTTTCTAGTGCTGCGCTGGGTGATC
CGCGTCGTACCGCGCGTCTGGTGAATGTTGCGGCGCAACTGGCCAAATAT
AGCGGCAAAAGCATTACCATTAGCAGCGAAGGCAGCAAAGCCATGCAGGA
AGGCGCGTATCGTTTTATTCGTAATCCGAACGTGAGCGCGGAAGCGATTC
GTAAAGCGGGTGCCATGCAGACCGTGAAACTGGCCCAGGAATTTCCGGAA
CTGCTGGCAATTGAAGATACCACCTCTCTGAGCTATCGTCATCAGGTGGC
GGAAGAACTGGGCAAACTGGGTAGCATTCAGGATAAAAGCCGTGGTTGGT
GGGTGCATAGCGTGCTGCTGCTGGAAGCGACCACCTTTCGTACCGTGGGC
CTGCTGCATCAAGAATGGTGGATGCGTCCGGATGATCCGGCGGATGCGGA
TGAAAAAGAAAGCGGCAAATGGCTGGCCGCTGCTGCAACTTCGCGTCTGA
GAATGGGCAGCATGATGAGCAACGTGATTGCGGTGTGCGATCGTGAAGCG
GATATTCATGCGTATCTGCAAGATAAACTGGCCCATAACGAACGTTTTGT
GGTGCGTAGCAAACATCCGCGTAAAGATGTGGAAAGCGGCCTGTATCTGT
ATGATCACCTGAAAAACCAGCCGGAACTGGGCGGCTATCAGATTAGCATT
CCGCAGAAAGGCGTGGTGGATAAACGTGGCAAACGTAAAAACCGTCCGGC
GCGTAAAGCGAGCCTGAGCCTGCGTAGCGGCCGTATTACCCTGAAACAGG
GCAACATTACCCTGAACGCGGTGCTGGCCGAAGAAATTAATCCGCCGAAA
GGCGAAACCCCGCTGAAATGGCTGCTGCTGACCAGCGAGCCGGTGGAAAG
TCTGGCCCAAGCGCTGCGTGTGATTGATATTTATACCCATCGTTGGCGCA
TTGAAGAATTTCACAAAGCGTGGAAAACGGGTGCGGGTGCGGAACGTCAG
CGTATGGAAGAACCGGATAACCTGGAACGTATGGTGAGCATTCTGAGCTT
TGTGGCGGTGCGTCTGCTGCAACTGCGTGAATCTTTTACTCCGCCGCAAG
CACTGCGTGCGCAGGGCCTGCTGAAAGAAGCGGAACACGTTGAAAGCCAG
AGCGCGGAAACCGTGCTGACCCCGGATGAATGCCAACTGCTGGGCTATCT
GGATAAAGGCAAACGCAAACGCAAAGAAAAAGCGGGCAGCCTGCAATGGG
CGTATATGGCGATTGCGCGTCTGGGCGGCTTTATGGATAGCAAACGTACC
GGCATTGCGAGCTGGGGTGCGCTGTGGGAAGGTTGGGAAGCGCTGCAAAG
CAAACTGGATGGCTTTCTGGCCGCGAAAGACCTGATGGCGCAGGGCATTA
AAATCTGC

A further illustrative example of an engineered transposase (TnH #4) amino acid sequence is shown as SEQ ID NO: 7.

SEQ ID NO: 7
MGKKTKRTADSSSSEDEEEYVVEKVLDRRVVKGQVEYLLKWKGFSEEHNT
WEPEKNLDCPELISEFMKKYKKMKEGENNKPREKSESNKRKSNFSNSADD
IKSKKKREQSNDTGSTGSTGSTGSTGSHMITSALHRAADWAKSVFSSAAL
GDPRRTARLVNVAAQLAKYSGKSITISSEGSKAMQEGAYRFIRNPNVSAE
AIRKAGAMQTVKLAQEFPELLAIEDTTSLSYRHQVAEELGKLGSIQDKSR
GWWVHSVLLLEATTFRTVGLLHQEWWMRPDDPADADEKESGKWLAAAATS
RLRMGSMMSNVIAVCDREADIHAYLQDKLAHNERFVVRSKHPRKDVESGL
YLYDHLKNQPELGGYQISIPQKGVVDKRGKRKNRPARKASLSLRSGRITL
KQGNITLNAVLAEEINPPKGETPLKWLLLTSEPVESLAQALRVIDIYTHR
WRIEEFHKAWKTGAGAERQRMEEPDNLERMVSILSFVAVRLLQLRESFTP
PQALRAQGLLKEAEHVESQSAETVLTPDECQLLGYLDKGKRKRKEKAGSL
QWAYMAIARLGGFMDSKRTGIASWGALWEGWEALQSKLDGFLAAKDLMAQ
GIKIC

An illustrative example of a nucleic acid sequence encoding an engineered transposase (TnH #4) is shown as SEQ ID NO: 8.

SEQ ID NO: 8
ATGGGAAAGAAAACCAAGCGGACAGCTGACAGTTCTTCTTCAGAGGATGA
GGAGGAGTATGTTGTGGAGAAGGTGCTAGACAGGCGCGTGGTTAAGGGAC
AAGTGGAATATCTACTGAAGTGGAAAGGCTTTTCTGAGGAGCACAATACT
TGGGAACCTGAGAAAAACTTGGATTGCCCTGAGCTAATTTCTGAATTTAT
GAAAAAGTATAAGAAGATGAAGGAGGGTGAAAATAATAAACCCAGGGAGA
AGTCAGAAAGTAACAAGAGGAAATCCAATTTCTCAAACAGTGCCGATGAC
ATCAAATCTAAAAAAAAGAGAGAGCAGAGCAATGATACGGGGTCGACCGG
CTCAACTGGTTCCACCGGGTCCACGGGCTCGCATATGATTACCAGTGCAC
TGCATCGTGCGGCGGATTGGGCGAAAAGCGTGTTTTCTAGTGCTGCGCTG
GGTGATCCGCGTCGTACCGCGCGTCTGGTGAATGTTGCGGCGCAACTGGC
CAAATATAGCGGCAAAAGCATTACCATTAGCAGCGAAGGCAGCAAAGCCA
TGCAGGAAGGCGCGTATCGTTTTATTCGTAATCCGAACGTGAGCGCGGAA
GCGATTCGTAAAGCGGGTGCCATGCAGACCGTGAAACTGGCCCAGGAATT
TCCGGAACTGCTGGCAATTGAAGATACCACCTCTCTGAGCTATCGTCATC
AGGTGGCGGAAGAACTGGGCAAACTGGGTAGCATTCAGGATAAAAGCCGT
GGTTGGTGGGTGCATAGCGTGCTGCTGCTGGAAGCGACCACCTTTCGTAC
CGTGGGCCTGCTGCATCAAGAATGGTGGATGCGTCCGGATGATCCGGCGG
ATGCGGATGAAAAAGAAAGCGGCAAATGGCTGGCCGCTGCTGCAACTTCG
CGTCTGAGAATGGGCAGCATGATGAGCAACGTGATTGCGGTGTGCGATCG
TGAAGCGGATATTCATGCGTATCTGCAAGATAAACTGGCCCATAACGAAC
GTTTTGTGGTGCGTAGCAAACATCCGCGTAAAGATGTGGAAAGCGGCCTG
TATCTGTATGATCACCTGAAAAACCAGCCGGAACTGGGCGGCTATCAGAT
TAGCATTCCGCAGAAAGGCGTGGTGGATAAACGTGGCAAACGTAAAAACC
GTCCGGCGCGTAAAGCGAGCCTGAGCCTGCGTAGCGGCCGTATTACCCTG
AAACAGGGCAACATTACCCTGAACGCGGTGCTGGCCGAAGAAATTAATCC
GCCGAAAGGCGAAACCCCGCTGAAATGGCTGCTGCTGACCAGCGAGCCGG
TGGAAAGTCTGGCCCAAGCGCTGCGTGTGATTGATATTTATACCCATCGT
TGGCGCATTGAAGAATTTCACAAAGCGTGGAAAACGGGTGCGGGTGCGGA
ACGTCAGCGTATGGAAGAACCGGATAACCTGGAACGTATGGTGAGCATTC
TGAGCTTTGTGGCGGTGCGTCTGCTGCAACTGCGTGAATCTTTTACTCCG
CCGCAAGCACTGCGTGCGCAGGGCCTGCTGAAAGAAGCGGAACACGTTGA
AAGCCAGAGCGCGGAAACCGTGCTGACCCCGGATGAATGCCAACTGCTGG
GCTATCTGGATAAAGGCAAACGCAAACGCAAAGAAAAAGCGGGCAGCCTG
CAATGGGCGTATATGGCGATTGCGCGTCTGGGCGGCTTTATGGATAGCAA
ACGTACCGGCATTGCGAGCTGGGGTGCGCTGTGGGAAGGTTGGGAAGCGC
TGCAAAGCAAACTGGATGGCTTTCTGGCCGCGAAAGACCTGATGGCGCAG
GGCATTAAAATCTGC

In one embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 1, SEQ ID NO: 3, SEQ ID NO: 5 or SEQ ID NO: 7.

In one embodiment, the engineered transposase comprises a sequence as set forth in SEQ ID NO: 1, SEQ ID NO: 3, SEQ ID NO: 5 or SEQ ID NO: 7.

In a preferred embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 1.

In a preferred embodiment, the engineered transposase comprises a sequence as set forth in SEQ ID NO: 1.

In one embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 3.

In one embodiment, the engineered transposase comprises a sequence as set forth in SEQ ID NO: 3.

In one embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 5.

In one embodiment, the engineered transposase comprises a sequence as set forth in SEQ ID NO: 5.

In one embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 7.

In one embodiment, the engineered transposase comprises a sequence as set forth in SEQ ID NO: 7.

In one embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 2, SEQ ID NO: 4, SEQ ID NO: 6 or SEQ ID NO: 8.

In one embodiment, the engineered transposase is encoded by a nucleic acid sequence comprising a sequence as set forth in SEQ ID NO: 2, SEQ ID NO: 4, SEQ ID NO: 6 or SEQ ID NO: 8.

In a preferred embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 2.

In a preferred embodiment, the engineered transposase is encoded by a nucleic acid sequence comprising a sequence as set forth in SEQ ID NO: 2.

In one embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 4.

In one embodiment, the engineered transposase is encoded by a nucleic acid sequence comprising a sequence as set forth in SEQ ID NO: 4.

In one embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 6.

In one embodiment, the engineered transposase is encoded by a nucleic acid sequence comprising a sequence as set forth in SEQ ID NO: 6.

In one embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 8.

In one embodiment, the engineered transposase is encoded by a nucleic acid sequence comprising a sequence as set forth in SEQ ID NO: 8.

### Transposase

A transposon (also known as a transposable element or a mobile genetic element) is a discrete DNA segment that is able to move from one location to another within a DNA sequence, such as a genome, in the absence of a complementary sequence in the DNA sequence (e.g. the genome). The mobilization of transposons is termed transposition and is catalysed by an enzyme called a transposase. DNA transposons are useful tools to analyze the regulatory genome, study embryonic development, identify genes and pathways implicated in disease or pathogenesis of pathogens, and even contribute to gene therapy. More recently, related in vitro applications have also been developed, including transposase-assisted chromatin immunoprecipitation sequencing (TAM-ChIP sequencing) and CUT & TAG.

Transposases may carry a ribonuclease-like catalytic domain and can use the same target site to catalyse both DNA cleavage and DNA strand transfer. Transposases are active when assembled into a synaptic complex (transposome) on the DNA.

As used herein, the term “transposon” refers to a DNA sequence that can undergo transposition.

As used herein, the term “transposase” may refer to an enzyme which catalyses the transposition of a transposon. Suitably, a transposase is an enzyme that is able to bind to the end of a transposon sequence and move it to other parts of the genome.

As used herein, the term “transposome” may refer to a transposon: transposase complex.

At least five families of transposases have been classified to date. These families use distinct catalytic mechanisms for break/rejoining of DNA. The present invention is not limited to any mechanism of transposition. Thus, any transposase may be employed in the present invention. Methods for producing a recombinant transposase are known in the art (see, for example, Reinius, B. et al. (2014) Genome Res., 24:2033-2040).

DDE transposases carry a triad of conserved amino acids—aspartate (D), aspartate (D) and glutamate (E)—which are required for the coordination of a metal ion required for catalysis. DDE transposases employ a cut-and-paste mechanism of transposition. Examples include the maize Ac transposon, as well as the Drosophila P element, bacteriophage Mu, Tn5, Sleeping Beauty, Tn10, Mariner, IS10, and IS50.

Tyrosine (Y) transposases also use a cut-and-paste mechanism of transposition, but employ a site-specific tyrosine residue. The transposon is excised from its original site (which is repaired); the transposon then forms a closed circle of DNA, which is integrated into a new site by a reversal of the original excision step. These transposons are usually found only in bacteria. Examples include Kangaroo, Tn916, and DIRS1.

Serine(S) transposases use a cut-and-paste (cut-out/paste-in) mechanism of transposition involving a circular DNA intermediate, which is similar to that of tyrosine transposases, only they employ a site-specific serine residue. These transposons are usually found only in bacteria. Examples include Tn5397 and IS607.

Rolling-circle (RC; or Y2) transposases may employ a copy-in mechanism, where the transposase copies a single strand directly into the target site by DNA replication, so that the old (template) and new (copied) transposons both have one newly synthesized strand. These transposons usually employ host DNA replication enzymes. Examples include IS91 and helitrons.

Reverse transcriptases/endonucleaseses (RT/En) catalyse the transposition of retrotransposons. Retrotransposons can vary in their mechanism of transposition. Some use the RT/En method, employing an endonuclease to nick the target site DNA, the nick serving as a primer for reverse transcription of an RNA copy by the reverse transcriptase enzyme. Examples include LINE-1 and TP-retrotransposons.

In some embodiments, the engineered transposase comprises a DD[E/D] (e.g. DDE) transposase. Suitably, the engineered transposase may comprise a transposase selected from Tn5, Sleeping Beauty, Tn10, Drosophila P element, bacteriophage Mu, Tc1/Mariner, IS10, and IS50 transposons. Preferably, the transposase is Tn5 or Sleeping Beauty. Suitably, the transposase may be a hyperactive transposase, such as the Nextera Tn5 transposase. The hyperactive Tn5 transposome complex (comprising a mutated recombinant Tn5 transposase enzyme with two synthetic oligonucleotides containing optimized 19 bp transposase recognition sites) exhibits 1,000 fold greater activity than wild type Tn5.

In a preferred embodiment, the engineered transposase comprises Tn5.

An illustrative example of a Tn5 amino acid sequence is shown as SEQ ID NO: 9.

SEQ ID NO: 9
MITSALHRAADWAKSVESSAALGDPRRTARLVNVAAQLAKYSGKSITISS
EGSKAMQEGAYRFIRNPNVSAEAIRKAGAMQTVKLAQEFPELLAIEDTTS
LSYRHQVAEELGKLGSIQDKSRGWWVHSVLLLEATTFRTVGLLHQEWWMR
PDDPADADEKESGKWLAAAATSRLRMGSMMSNVIAVCDREADIHAYLQDK
LAHNERFVVRSKHPRKDVESGLYLYDHLKNQPELGGYQISIPQKGVVDKR
GKRKNRPARKASLSLRSGRITLKQGNITLNAVLAEEINPPKGETPLKWLL
LTSEPVESLAQALRVIDIYTHRWRIEEFHKAWKTGAGAERQRMEEPDNLE
RMVSILSFVAVRLLQLRESFTPPQALRAQGLLKEAEHVESQSAETVLTPD
ECQLLGYLDKGKRKRKEKAGSLQWAYMAIARLGGFMDSKRTGIASWGALW
EGWEALQSKLDGFLAAKDLMAQGIKI

An illustrative example of a nucleic acid sequence encoding Tn5 is shown as SEQ ID NO: 10.

SEQ ID NO: 10
ATGATTACCAGTGCACTGCATCGTGCGGCGGATTGGGCGAAAAGCGTGTT
TTCTAGTGCTGCGCTGGGTGATCCGCGTCGTACCGCGCGTCTGGTGAATG
TTGCGGCGCAACTGGCCAAATATAGCGGCAAAAGCATTACCATTAGCAGC
GAAGGCAGCAAAGCCATGCAGGAAGGCGCGTATCGTTTTATTCGTAATCC
GAACGTGAGCGCGGAAGCGATTCGTAAAGCGGGTGCCATGCAGACCGTGA
AACTGGCCCAGGAATTTCCGGAACTGCTGGCAATTGAAGATACCACCTCT
CTGAGCTATCGTCATCAGGTGGCGGAAGAACTGGGCAAACTGGGTAGCAT
TCAGGATAAAAGCCGTGGTTGGTGGGTGCATAGCGTGCTGCTGCTGGAAG
CGACCACCTTTCGTACCGTGGGCCTGCTGCATCAAGAATGGTGGATGCGT
CCGGATGATCCGGCGGATGCGGATGAAAAAGAAAGCGGCAAATGGCTGGC
CGCTGCTGCAACTTCGCGTCTGAGAATGGGCAGCATGATGAGCAACGTGA
TTGCGGTGTGCGATCGTGAAGCGGATATTCATGCGTATCTGCAAGATAAA
CTGGCCCATAACGAACGTTTTGTGGTGCGTAGCAAACATCCGCGTAAAGA
TGTGGAAAGCGGCCTGTATCTGTATGATCACCTGAAAAACCAGCCGGAAC
TGGGCGGCTATCAGATTAGCATTCCGCAGAAAGGCGTGGTGGATAAACGT
GGCAAACGTAAAAACCGTCCGGCGCGTAAAGCGAGCCTGAGCCTGCGTAG
CGGCCGTATTACCCTGAAACAGGGCAACATTACCCTGAACGCGGTGCTGG
CCGAAGAAATTAATCCGCCGAAAGGCGAAACCCCGCTGAAATGGCTGCTG
CTGACCAGCGAGCCGGTGGAAAGTCTGGCCCAAGCGCTGCGTGTGATTGA
TATTTATACCCATCGTTGGCGCATTGAAGAATTTCACAAAGCGTGGAAAA
CGGGTGCGGGTGCGGAACGTCAGCGTATGGAAGAACCGGATAACCTGGAA
CGTATGGTGAGCATTCTGAGCTTTGTGGCGGTGCGTCTGCTGCAACTGCG
TGAATCTTTTACTCCGCCGCAAGCACTGCGTGCGCAGGGCCTGCTGAAAG
AAGCGGAACACGTTGAAAGCCAGAGCGCGGAAACCGTGCTGACCCCGGAT
GAATGCCAACTGCTGGGCTATCTGGATAAAGGCAAACGCAAACGCAAAGA
AAAAGCGGGCAGCCTGCAATGGGCGTATATGGCGATTGCGCGTCTGGGCG
GCTTTATGGATAGCAAACGTACCGGCATTGCGAGCTGGGGTGCGCTGTGG
GAAGGTTGGGAAGCGCTGCAAAGCAAACTGGATGGCTTTCTGGCCGCGAA
AGACCTGATGGCGCAGGGCATTAAAATC

In one embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 9.

In one embodiment, the engineered transposase comprises a sequence as set forth in SEQ ID NO: 9.

In one embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 10.

In one embodiment, the engineered transposase comprises a sequence as set forth in SEQ ID NO: 10.

The transposase is operably linked to a polypeptide which binds to a component of chromatin (e.g. of heterochromatin).

As used herein, the term “operably linked” means that parts (e.g. the transposase and the polypeptide that binds to a component of heterochromatin) are linked together in a manner which enables both to carry out their function substantially unhindered. Suitably, the transposase may be conjugated to the polypeptide that binds to a component of heterochromatin or fused to the polypeptide that binds to a component of heterochromatin (e.g. the transposase and polypeptide that binds to a component of heterochromatin may be a fusion protein). Conjugation may be performed using methods known in the art, for example using a chemical cross-linking agent.

In a preferred embodiment, the transposase is fused to the polypeptide that binds to a component of heterochromatin. Suitably, the N-terminus of the transposase may be fused to the polypeptide that binds to a component of heterochromatin. The transposase may be fused to the polypeptide by a linker sequence.

In a preferred embodiment, the transposase and polypeptide that binds to a component of heterochromatin are a fusion protein (e.g. form a single amino acid chain). Suitably, the N-terminus of the transposase may be joined to the polypeptide that binds to a component of heterochromatin via one or more peptide bond. The transposase may be joined to the polypeptide that binds to a component of heterochromatin by a linker sequence.

Suitable linker sequences for fusing two domains are known in the art.

Suitably, the linker may be a single amino acid, e.g. proline, which is suitable to separate the peptides. Suitably, the transposase and polypeptide that binds to a component of heterochromatin may be coupled by a flexible linker peptide.

Illustrative flexible linker peptides are glycine and/or serine-rich peptides. Suitably, the linker may comprise one or more glycine, serine and/or threonine residue. The peptide linker may comprise 4-20, 4-15, 4-10, 8-20 or 8-15 amino acids. The peptide linker may comprise a 3 to 5 poly-tyrosine-glycine-serine (TGS) linker (i.e. a 3× to 5×TGS repeat). Examples of suitable peptide linkers include, but are not limited to, TGSTGSTGS (SEQ ID NO: 11), TGSTGSTGSTGS (SEQ ID NO: 12), TGSTGSTGSTGSTGS (SEQ ID NO: 13), GGSGGS (SEQ ID NO: 14), SGSGSGS (SEQ ID NO: 15), GGGGSGGGGS (SEQ ID NO: 16), GSGSGSGSGS (SEQ ID NO: 17), GGSGGSGGSGGS (SEQ ID NO: 18), GGGGGGGGSGGGGS (SEQ ID NO: 19) and SDP.

Preferably, the linker sequence has the amino acid sequence TGSTGSTGS (SEQ ID NO: 11), or TGSTGSTGSTGSTGS (SEQ ID NO: 13).

### Polypeptide

Chromatin is a highly organised complex of DNA and protein found in the nucleus of eukaryotic cells. The basic structural unit of chromatin is the nucleosome, which consists of a section of DNA (approximately 147 base pairs) wound around an octamer of histones containing two units of each histone H2A, H2B, H3, and H4. DNA may be less tightly compacted in a structure known as euchromatin (also termed “open” chromatin), whilst other regions of DNA are generally more condensed and associated with structural proteins in a structure known as heterochromatin (also termed “closed” chromatin and compacted chromatin). Heterochromatin is assembled and maintained through the tri-methylation of the histone residue H3K9 (i.e. H3K9me3) and its accurate regulation is essential for cells, for example, in the definition of cell identity and the maintenance of genomic integrity. Heterochromatin encompasses up to half of the entire genome and harbours and regulates a large array of transposable elements and ncRNAs.

Histones are the major protein components of chromatin and are small basic proteins with a flexible amino-terminal “tail”. A variety of histone-modifying enzymes are responsible for a multiplicity of post-translational modifications on specific serine, lysine, and arginine residues within the flexible amino-terminal histone tail. The methylation of lysine residues on histones H3 and H4 is well-characterised. Histone methylation may be either associated with transcriptional activation (for example, methylation of H3K4, H3K36, and H3K79) or associated with transcriptional repression (for example, methylation of H3K9, H3K27 and H4K20) depending on which amino acid residue is modified and to what extent (monomethylation, dimethylation, or trimethylation) the residue is modified. Tri-methylation of the histone residue H3K9 (i.e. H3K9me3) leads to the assembly of heterochromatin.

The polypeptide may bind to a component of euchromatin.

In a preferred embodiment, the polypeptide binds to a component of heterochromatin.

As used herein, the term “a component of chromatin” refers to a species (preferably a protein species) present within the chromatin structure. Preferably, the component of chromatin (e.g. of heterochromatin) may be a histone protein, such as a methylated histone.

The polypeptide may bind to a component of chromatin (e.g. of heterochromatin) which is associated with transcriptional activation. Suitably, the polypeptide may bind to a methylated histone which is associated with transcriptional activation. Suitably, the polypeptide may bind to an acetylated histone which is associated with transcriptional activation. For example, the acetylated histone may be H3K27Ac. Domains which bind to acetylated histones are known in the art. For example, bromodomains bind to H3K27Ac.

Preferably, the polypeptide may bind to a component of chromatin (e.g. of heterochromatin) which is associated with transcriptional repression. Suitably, the polypeptide may bind to a methylated histone which is associated with transcriptional repression. For example, the methylated histone may be H3K9me3 and/or H3K27me3.

Suitably, the polypeptide may bind to a methylated histone which is associated with gene bodies and alternative splicing events. For example, the methylated histone may be H3K36me3.

Domains which bind to methylated histones are known in the art. For example, the chromodomain of chromobox protein homolog 8 (CBX8) and JmJc domains bind to H3K27me3, the chromodomain of heterochromatin protein 1-α binds to H3K9me3 and the chromodomains of yeast protein Eaf3 and of CBX5 bind to H3K36me3.

In one embodiment, the polypeptide binds to H3K27Ac, H3K9me3, H3K27me3 and/or H3K36me3.

In a preferred embodiment, the polypeptide binds to H3K9me3.

Numerous binding domains that recognise a component of chromatin (e.g. of heterochromatin) are known in the art. For example, the polypeptide may comprise a chromodomain, a bromodomain, a JmJc domain, a HMG-box domain, a KRAB domain or a PWWP domain. Suitably, the polypeptide may comprise the bromodomain of BRD4, the JmJc domain of KDM6B, the HMG-box domain of HMGB1, the KRAB domain of SSX6P or the PWWP domain of DNMT3a or the PWWP domain of DNMT3b. Preferably, the polypeptide does not comprise an antibody or an antibody binding domain.

The chromodomain may be, for example, a chromodomain of a chromobox protein homolog (CBX).

The chromodomain may be, for example, selected from the chromodomain of heterochromatin protein 1-α, of CBX8, of yeast protein Eaf3, of CBX5, of CBX2, of CBX7 or of M phase phosphoprotein 8.

The chromodomain may be, for example, selected from the chromodomain of heterochromatin protein 1-α, of CBX8, of yeast protein Eaf3 or of CBX5.

In one preferred embodiment, the polypeptide comprises the chromodomain of heterochromatin protein 1-α. Heterochromatin protein 1-α is one of the proteins involved in heterochromatin assembly and maintenance, and specifically (e.g. preferentially) binds to H3K9me3 via its chromodomain.

In one preferred embodiment, the polypeptide comprises the chromodomain of CBX5. CBX5 specifically (e.g. preferentially) binds to H3K36me3, which is associated with gene bodies and alternative splicing events, via its chromodomain.

In preferred embodiments, the engineered transposase comprises Tn5 operably linked to a chromodomain, preferably the chromodomain of heterochromatin protein 1-α.

An illustrative example of a heterochromatin protein 1-α amino acid sequence is shown as SEQ ID NO: 20.

SEQ ID NO: 20
MGKKTKRTADSSSSEDEEEYVVEKVLDRRVVKGQVEYLLKWKGFSEEHNT
WEPEKNLDCPELISEFMKKYKKMKEGENNKPREKSESNKRKSNESNSADD
IKSKKKREQSNDIARGFERGLEPEKIIGATDSCGDLMFLMKWKDTDEADL
VLAKEANVKCPQIVIAFYEERLTWHAYPEDAENKEKETAKS

An illustrative example of a nucleic acid sequence encoding heterochromatin protein 1-α is shown as SEQ ID NO: 21.

SEQ ID NO: 21
ATGGGAAAGAAAACCAAGCGGACAGCTGACAGTTCTTCTTCAGAGGATGA
GGAGGAGTATGTTGTGGAGAAGGTGCTAGACAGGCGCGTGGTTAAGGGAC
AAGTGGAATATCTACTGAAGTGGAAAGGCTTTTCTGAGGAGCACAATACT
TGGGAACCTGAGAAAAACTTGGATTGCCCTGAGCTAATTTCTGAATTTAT
GAAAAAGTATAAGAAGATGAAGGAGGGTGAAAATAATAAACCCAGGGAGA
AGTCAGAAAGTAACAAGAGGAAATCCAATTTCTCAAACAGTGCCGATGAC
ATCAAATCTAAAAAAAAGAGAGAGCAGAGCAATGATATCGCTCGGGGCTT
TGAGAGAGGACTGGAACCAGAAAAGATCATTGGGGCAACAGATTCCTGTG
GTGATTTAATGTTCCTAATGAAATGGAAAGACACAGATGAAGCTGACCTG
GTTCTTGCAAAAGAAGCTAATGTGAAATGTCCACAAATTGTGATAGCATT
TTATGAAGAGAGACTGACATGGCATGCATATCCTGAGGATGCGGAAAACA
AAGAGAAAGAAACAGCAAAGAGCTAA

An illustrative example of a heterochromatin protein 1-α chromodomain amino acid sequence (1-75aa chromodomain plus 37aa natural linker of HP1-α which connects the chromodomain with the chromoshadow domain) is shown as SEQ ID NO: 22.

SEQ ID NO: 22
MGKKTKRTADSSSSEDEEEYVVEKVLDRRVVKGQVEYLLKWKGFSEEHNT
WEPEKNLDCPELISEFMKKYKKMKEGENNKPREKSESNKRKSNFSNSADD
IKSKKKREQSND

An illustrative example of a nucleic acid sequence encoding heterochromatin protein 1-α chromodomain (1-75aa chromodomain plus 37aa natural linker of HP1-α) is shown as SEQ ID NO: 23.

SEQ ID NO: 23
ATGGGAAAGAAAACCAAGCGGACAGCTGACAGTTCTTCTTCAGAGGATGA
GGAGGAGTATGTTGTGGAGAAGGTGCTAGACAGGCGCGTGGTTAAGGGAC
AAGTGGAATATCTACTGAAGTGGAAAGGCTTTTCTGAGGAGCACAATACT
TGGGAACCTGAGAAAAACTTGGATTGCCCTGAGCTAATTTCTGAATTTAT
GAAAAAGTATAAGAAGATGAAGGAGGGTGAAAATAATAAACCCAGGGAGA
AGTCAGAAAGTAACAAGAGGAAATCCAATTTCTCAAACAGTGCCGATGAC
ATCAAATCTAAAAAAAAGAGAGAGCAGAGCAATGAT

An illustrative example of a heterochromatin protein 1-α chromodomain amino acid sequence (1-75aa chromodomain plus 18aa natural linker of HP1-α) is shown as SEQ ID NO: 24.

SEQ ID NO: 24
MGKKTKRTADSSSSEDEEEYVVEKVLDRRVVKGQVEYLLKWKGFSEEHNT
WEPEKNLDCPELISEFMKKYKKMKEGENNKPREKSESNKRKSN

An illustrative example of a nucleic acid sequence encoding heterochromatin protein 1-α chromodomain (1-75aa chromodomain plus 18aa natural linker of HP1-α) is shown as SEQ ID NO: 25.

SEQ ID NO: 25
ATGGGAAAGAAAACCAAGCGGACAGCTGACAGTTCTTCTTCAGAGGATGA
GGAGGAGTATGTTGTGGAGAAGGTGCTAGACAGGCGCGTGGTTAAGGGAC
AAGTGGAATATCTACTGAAGTGGAAAGGCTTTTCTGAGGAGCACAATACT
TGGGAACCTGAGAAAAACTTGGATTGCCCTGAGCTAATTTCTGAATTTAT
GAAAAAGTATAAGAAGATGAAGGAGGGTGAAAATAATAAACCCAGGGAGA
AGTCAGAAAGTAACAAGAGGAAATCCAAT

In one embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 20, SEQ ID NO: 22 or SEQ ID NO: 24.

In one embodiment, the engineered transposase comprises a sequence as set forth in SEQ ID NO: 20, SEQ ID NO: 22 or SEQ ID NO: 24.

In one embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 22.

In one embodiment, the engineered transposase comprises a sequence as set forth in SEQ ID NO: 22.

In one embodiment, the engineered transposase comprises a sequence having at least 70% (suitably, at least 80%, at least 85%, at least 90%, at least 95%, at least 96%, at least 97%, at least 98% or at least 99%) sequence identity to the sequence set forth in SEQ ID NO: 24.

In one embodiment, the engineered transposase comprises a sequence as set forth in SEQ ID NO: 24.

In one embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 21, SEQ ID NO: 23 or SEQ ID NO: 25.

In one embodiment, the engineered transposase is encoded by a nucleic acid sequence comprising a sequence as set forth in SEQ ID NO: 21, SEQ ID NO: 23 or SEQ ID NO: 25.

In one embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 23.

In one embodiment, the engineered transposase is encoded by a nucleic acid sequence comprising a sequence as set forth in SEQ ID NO: 23.

In one embodiment, the engineered transposase is encoded by a nucleic acid sequence comprising a sequence having at least 70% (suitably, at least 80%, at least 85%, at least 90%, at least 95%, at least 96%, at least 97%, at least 98% or at least 99%) sequence identity to the sequence set forth in SEQ ID NO: 25.

In one embodiment, the engineered transposase is encoded by a nucleic acid sequence comprising a sequence as set forth in SEQ ID NO: 25.

It is preferred that the polypeptide preferentially binds to one component of chromatin (e.g. of heterochromatin) as compared to other components of chromatin (e.g. of heterochromatin), i.e. that the polypeptide has a greater binding affinity for one component compared to its binding affinity for another component of chromatin (e.g. of heterochromatin). For example, the polypeptide may preferentially bind to H3K9me3 compared to H3K4me3. Thus, the polypeptide may have a greater binding affinity for H3K9me3 compared to H3K4me3 (e.g. a binding affinity for H3K9me3 of at least 10, 50, 100, 1000 or 10000 times that of its affinity to bind H3K4me3).

The polypeptide may have a high binding affinity for the component of chromatin (e.g. of heterochromatin), e.g. may have a Kd in the range of 10-5M, 10-6M, 10-7M or 10-9M or less. The polypeptide may have a binding affinity for the component of chromatin (e.g. of heterochromatin) that corresponds to a Kd of less than 30 nM, 20 nM, 15 nM or 10 nM, more preferably of less than 10, 9.5, 9, 8.5, 8, 7.5, 7, 6.5, 6, 5.5, 5, 4.5, 4, 3.5, 3, 2.5, 2, 1.5 or 1 nM, most preferably less than 0.9, 0.8, 0.7, 0.6, 0.5, 0.4, 0.3, 0.2 or 0.1 nM. Any appropriate method of determining Kd may be used, e.g. BIAcore analysis.

The polypeptide may preferentially bind to two components of chromatin (e.g. of heterochromatin) as compared to other components of chromatin (e.g. of heterochromatin), i.e. the polypeptide may have a greater binding affinity for the two components compared to other components of chromatin (e.g. of heterochromatin). For example, the polypeptide may have a binding affinity for each of the two components that is at least 10, 50, 100, 1000 or 10000 times that of its affinity to other components.

Suitable methods for determining binding will be known to those of skill in the art. For example, binding can be assessed by flow cytometry, immunohistochemistry, Western blotting, ELISA and surface plasmon resonance. It is within the ambit of the skilled person to select and implement a suitable assay to determine if a candidate polypeptide (e.g. a chromodomain) is capable of binding to a component of chromatin (e.g. a methylated histone). Suitably, the ability of the polypeptide to direct the transposase to the target component of chromatin may be determined as described herein (see Example 2).

### Engineered Transposome Complex

In a further aspect, the present invention provides an engineered transposome complex comprising an oligonucleotide and an engineered transposase as described herein.

The oligonucleotide may comprise a transposase recognition site mosaic end (ME). Suitably, when the transposon is Tn5, the ME may comprise the sequence AGATGTGTATAAGAGACAG (SEQ ID NO: 26).

As used herein, the term “mosaic end” refers to a transposase recognition site mosaic end (ME). The ME sequence may be required by the transposase for catalysis of the transposition reaction.

Suitably, the oligonucleotide may be from 1 to 100, from 1 to 50 or from 1 to 20 nucleotides in length.

For NGS applications, the oligonucleotide may further comprise a sequencing adaptor. Suitably, the sequencing adaptor may be an NGS platform-specific tag required for sequencing. Preferably, the sequencing adaptor is a sequencing primer.

For multiplexed sequencing applications, the oligonucleotide may further comprise a unique tagging sequence (also termed a barcode sequence). Suitably, the tagging sequence uniquely labels the oligonucleotide species so that it can be distinguished from other oligonucleotide species in the reaction (which may correspond to further transposome complexes) for identification in multiplexed sequencing applications in which multiple transposome complexes are used simultaneously with a single sample. The tagging sequence may be a short nucleotide sequence. Suitably, the tagging sequence may be less than 20, less than 10 or 8 bases in length. Preferably, the tagging sequence is 8 bases in length.

In one embodiment, the oligonucleotide comprises a sequencing primer site, a tagging sequence and a mosaic end.

In some embodiments, the oligonucleotide comprises a 5′ phosphate group. Suitably, the 5′ phosphate group facilitates binding of the oligonucleotide (and thereby binding of a tagged DNA sequence) to a capture moiety, e.g. a bead, such as a hydrogel bead.

The oligonucleotide may for example comprise a sequence as set forth in SEQ ID NO: 27, SEQ ID NO: 28, SEQ ID NO: 29, SEQ ID NO: 30, SEQ ID NO: 31, SEQ ID NO: 32, SEQ ID NO: 33 or SEQ ID NO: 34.

Methods for assembling the transposome complex, i.e. loading the oligonucleotide onto a transposase, such as an engineered transposase as described herein, are known in the art (see, for example, Reinius, B. et al. (2014) Genome Res., 24:2033-2040).

### Methods and Uses

The present invention provides methods for tagging genomic DNA (e.g. chromatin) for sequencing applications. Generally, the methods may comprise preparing engineered transposome complexes containing sequencing adaptors with an engineered transposase that binds to a component of chromatin. The complexes may be added to a sample comprising genomic DNA such that the engineered transposase binds to the component of chromatin. Tagmentation by the engineered transposase of the genomic DNA surrounding the binding site then occurs. Thus, the genomic DNA is fragmented and tagged with the sequencing adaptor to form a sequencing-ready library. The library may subsequently be sequenced.

The methods of the invention may employ an engineered transposome complex which binds to heterochromatin or which binds to distinct regions of chromatin, e.g. to euchromatin and to heterochromatin. Thus, this approach covers a large portion of the genome inaccessible to approaches surveying accessible chromatin to obtain a comprehensive perspective on the epigenetic and genomic landscape. A further advantage of this approach is that it is applicable to single cell analysis.

### Method for DNA Sequencing and Optionally RNA Sequencing

In a further aspect, the present invention provides a method for DNA sequencing comprising the steps:
a) providing a sample comprising genomic DNA;
b) adding at least one engineered transposase as described herein;
c) amplifying tagged DNA;
d) optionally isolating the amplified DNA; and
e) sequencing tagged DNA, the amplified DNA or the isolated DNA.

In a further aspect, the present invention provides a method for DNA sequencing comprising the steps:
a) providing a sample comprising genomic DNA;
b) adding at least one engineered transposome complex as described herein;
c) amplifying tagged DNA;
d) optionally isolating the amplified DNA; and
e) sequencing tagged DNA, amplified DNA or the isolated DNA.

In a further aspect, the invention provides a method for DNA sequencing and RNA sequencing comprising the steps:
a) providing a sample comprising genomic DNA and RNA;
b) (i) adding at least one engineered transposome complex as described herein; and
   (ii) tagging the RNA;
c) optionally amplifying tagged DNA and/or tagged RNA;
d) optionally isolating the amplified DNA and/or the amplified cDNA; and
e) sequencing tagged DNA, the amplified DNA or the isolated DNA and sequencing tagged RNA, the amplified cDNA or the isolated cDNA.

One embodiment of the methods of the invention (termed “genome and epigenome by transposases sequencing” or “GET-seq”) is a method which improves the methods currently used for DNA sequencing applications. GET-seq may employ two different transposome complexes which bind to distinct regions of chromatin, e.g. to euchromatin and to heterochromatin. Thus, this approach covers a large portion of the genome inaccessible to approaches surveying accessible chromatin to obtain a comprehensive and dynamic perspective on the epigenetic and genomic landscape. A further advantage of this approach is that it is applicable to single cell analysis, termed “single cell genome and epigenome by transposases sequencing” or “scGET-seq”.

Another embodiment of the methods of the invention (“GET²-seq”) is a method which improves the methods currently used for combined (e.g. simultaneous) DNA sequencing and RNA sequencing applications. GET²-seq is based upon GET-seq. Thus, similarly to GET-seq, GET²-seq may employ two different transposome complexes which bind to distinct regions of chromatin, e.g. to euchromatin and to heterochromatin. Thus, this approach also allows to obtain a comprehensive and dynamic perspective on the epigenetic and genomic landscape and is applicable to single cell analysis, termed “single cell genome and epigenome by transposases sequencing” or “scGET²-seq”. A further advantage of this approach is that it combines DNA sequencing with RNA sequencing.

Accordingly, in one embodiment, step b) further comprises adding at least one further transposome complex.

In a further aspect, the invention provides a method for DNA sequencing comprising the steps:
a) providing a sample comprising genomic DNA;
b) adding at least one engineered transposome complex as described herein and at least one further transposome complex as described herein;
c) amplifying tagged DNA;
d) optionally isolating the amplified DNA; and
e) sequencing tagged DNA, the amplified DNA or the isolated DNA.

In a further aspect, the invention provides a method for DNA sequencing and RNA sequencing comprising the steps:
a) providing a sample comprising genomic DNA and RNA;
b) (i) adding at least one engineered transposome complex as described herein and at least one further transposome complex as described herein; and
   (ii) tagging the RNA;
c) optionally amplifying tagged DNA and/or tagged RNA;
d) optionally isolating the amplified DNA and/or the amplified cDNA; and
e) sequencing tagged DNA, the amplified DNA or the isolated DNA and sequencing tagged RNA, the amplified cDNA or the isolated cDNA.

Preferably, the at least one further transposome complex binds to a different component of chromatin (e.g. of heterochromatin) to the at least one engineered transposome complex. Suitably, the at least one further transposome complex binds to a distinct region of chromatin to the first transposome complex, i.e. the at least one engineered transposome complex and the at least one further transposome complex may differentially bind to a component of open chromatin and to a component of condensed chromatin.

In a preferred embodiment, the at least one further transposome complex and the at least one engineered transposome complex have overlapping, but not identical, binding specificity. Suitably, both transposome complexes bind to one region of chromatin and the at least one further transposome complex additionally binds to a distinct region of chromatin to the first transposome complex, e.g. the at least one engineered transposome complex and the at least one further transposome complex may both bind to a component of open chromatin and differentially bind to a component of condensed chromatin.

Any suitable further transposome complex may be added. Suitable transposome complexes are known in the art. For example, the at least one further transposome complex may comprise Tn5, such as a hyperactive Tn5 transposase (e.g. the Nextera Tn5 transposase). Suitably, the at least one further transposome complex may comprise an engineered transposome complex as described herein. The engineered additional transposases, e.g. including domains targeting other portions of the genome, may extend and integrate the information provided by TnH.

In one embodiment, the at least one engineered transposome complex and the at least one further transposome complex may each bind (e.g. preferentially bind) to a different methylated histone. In one embodiment, the at least one engineered transposome complex and the at least one further transposome complex may each have a different methylated histone binding specificity. For example, the at least one engineered transposome complex may bind (e.g. preferentially bind) to H3K9me3 and the at least one further transposome complex may bind (e.g. preferentially bind) to H3K4me3. In one embodiment, the two transposome complexes have overlapping, but not identical, binding specificity. For example, the at least one engineered transposome complex may bind (e.g. preferentially bind) to both H3K9me3 and H3K4me3, and the at least one further transposome complex may bind (e.g. preferentially bind) to H3K4me3. Thus, simultaneous analysis of both open and condensed chromatin may be performed using the methods of the invention.

Suitably, the at least one engineered transposome complex and the at least one further transposome complex may be added simultaneously or sequentially. Preferably, the at least one engineered transposome complex and the at least one further transposome complex are added sequentially. More preferably, the at least one engineered transposome complex is added following the addition of the at least one further transposome complex. The ratio of the at least one engineered transposome complex to the at least one further transposome complex which is added to the genomic DNA may be varied. Suitably, the ratio of the at least one engineered transposome complex to the at least one further transposome complex may be varied from 1:99 to 99:1 (suitably, 5:95, 10:90, 25:75, 50:50, 75:25, 90:10 or 95:5).

The term “tagging sequence” is used interchangeably herein with the term “identifier sequence” to refer to a short sequence that can be added to a primer or otherwise included in the oligonucleotide or otherwise used as label to provide a unique identifier. Such an identifier sequence (or tag) can be a unique base sequence of varying but defined length, typically from 4-16 bp used for identifying a specific nucleic acid sample. Identifier sequences are useful according to the invention, as by using such identifier sequence, the origin of a (PCR) sample can be determined upon further processing. In the case of combining processed products originating from different nucleic acid samples, the different nucleic acid samples may be identified using different identifier sequences, i.e. identifier sequences may then assist in identifying the sequences corresponding to the different samples. Identifier sequences preferably differ from each other by at least two base pairs and preferably do not contain two identical consecutive bases to prevent misreads.

In one embodiment, the tagging sequence of the at least one engineered transposome complex differs from the tagging sequence of the at least one further transposome complex. Thus, the methods of the invention may be used for multiplexed sequencing applications.

The step of tagging the RNA may be performed prior to, at the same time as or after the step of adding the at least one engineered transposase as described herein. Suitably, the step of tagging the RNA is performed after the step of adding the at least one engineered transposase as described herein.

The step of tagging the RNA may be performed prior to, at the same time as or after the step of adding the at least one engineered transposome complex as described herein. Suitably, the step of tagging the RNA is performed after the step of adding the at least one engineered transposome complex as described herein.

The step of tagging the RNA may be performed prior to, at the same time as or after the step of adding the at least one engineered transposome complex as described herein and at least one further transposome complex as described herein. Suitably, the step of tagging the RNA is performed after the step of adding the at least one engineered transposome complex as described herein and at least one further transposome complex as described herein.

The term “tagging the RNA” refers to the attachment of an RNA tagging sequence as described herein onto one end of an RNA sequence, e.g. to one end of RNA sequences within the sample. Suitably, tagging the RNA involves RNA capture and RNA tagging. For example, tagging the RNA may be performed using an RNA capture probe which further comprises an RNA tagging sequence. Suitably, the RNA capture probe may comprise a polyA capture probe. A capture probe may be a nucleotide sequence such as an oligonucleotide. Suitably, the RNA capture probe may be complexed with a bead, e.g. a hydrogel bead. Suitably, the RNA tagging sequence is attached to the 3′ end of mRNA molecules in the sample. Hence, the RNA tagging sequence as described herein may be complexed with one end (e.g. the 3′ end) of the RNA molecules in the sample to generate a compatible library (e.g. an NGS compatible library) for sequencing applications.

The term “RNA capture probe” may refer to a nucleotide sequence which is specific for RNA. Suitably, the RNA capture probe may comprise a nucleotide sequence which is complementary to the RNA sequence. In the context of the present invention, the RNA capture probe preferably further comprises an RNA tagging sequence as described herein and may be complexed with a hydrogel bead.

Suitably, the RNA capture probe is a polyA capture probe, i.e. comprises a nucleotide sequence which is specific for polyA. The polyA capture probe may comprise a nucleotide sequence which is complementary to polyA. In the context of the present invention, the polyA capture probe preferably further comprises an RNA tagging sequence as described herein and may be complexed with a hydrogel bead.

In some embodiments, tagging the RNA is performed using an RNA capture probe as described herein.

In some embodiments, tagging the RNA is performed using a polyA capture probe as described herein.

Methods for tagging RNA are known in the art. Tagging the RNA may be carried out using any suitable method, for example, the method disclosed herein (see Example 11).

In one embodiment, the RNA tagging sequence may be from 1 to 100, from 1 to 50 or from 1 to 20 nucleotides in length.

For sequencing (e.g. NGS or RNA-Seq) applications, the RNA tagging sequence may comprise a sequencing adaptor. Suitably, the sequencing adaptor may be an NGS platform-specific tag or RNA-Seq specific required for sequencing. Preferably, the sequencing adaptor is a sequencing primer.

For multiplexed sequencing applications, the RNA tagging sequence may further comprise a unique tagging sequence (also termed a barcode sequence). Suitably, the barcode sequence uniquely labels the RNA tagging sequence species so that it can be distinguished from other RNA tagging sequence species in the reaction for identification in multiplexed sequencing applications in which multiple RNA tagging sequences are used simultaneously with a single sample. The barcode sequence may be a short nucleotide sequence. Suitably, the barcode sequence may be less than 20, less than 10 or 8 bases in length. Preferably, the barcode sequence is 8 bases in length.

In one embodiment, the RNA tagging sequence comprises a sequencing adaptor (e.g. a sequencing primer site).

In one embodiment, the RNA tagging sequence comprises a barcode sequence.

In one embodiment, the RNA tagging sequence comprises a sequencing adaptor (e.g. a sequencing primer site) and a barcode sequence.

One embodiment of the methods of the invention (termed “Chromatin Velocity”) is a method which improves the methods currently used for DNA sequencing applications. Chromatin Velocity exploits the ratio between signals obtained from open vs condensed chromatin, at any given location, with an increase in this value pointing to a dynamic process leading to a more relaxed chromatin, while the opposite is indicative of chromatin compaction. Thus, Chromatin Velocity investigates developmental dynamics in terms of differential compaction of chromatin, i.e. captures single cell trajectories in terms of the overall direction and the velocity of chromatin remodelling. This permits the analysis of epigenetic transitions underlying crucial biological processes in health and disease.

In one embodiment, the signal obtained from the at least one further transposome complex and the at least one engineered transposome complex at a DNA locus may be compared.

“Amplifying” refers to a polynucleotide amplification reaction, namely, a population of polynucleotides that are replicated from one or more starting polynucleotides. Amplifying may refer to a variety of amplification reactions, including but not limited to polymerase chain reaction (PCR), linear polymerase reactions, nucleic acid sequence-based amplification, rolling circle amplification, reverse-transcriptase PCR (RT-PCR) and like reactions. RT-PCR uses RNA rather than DNA as the PCR template. RT-PCR involves the conversion of RNA molecules by reverse transcription into DNA molecules to yield complementary DNA (cDNA), followed by amplification the cDNA (e.g. universal amplification or amplification of specific cDNA targets) by PCR. In one embodiment, the amplifying RNA (e.g. the tagged RNA) is by RT-PCR.

“Sequencing” refers to determining the order of nucleotides (base sequences) in a nucleic acid sample, e.g. DNA or RNA. Many techniques are available such as Next Generation Sequencing (NGS), Sanger sequencing and High throughput sequencing technologies such as offered by Roche, Illumina and Applied Biosystems, as well as approaches such as Nanopore, pacBio and Ion Torrent. These techniques are also applicable for sequencing RNA (RNA-Seq) or cDNA-Seq (the sequencing of a cDNA library derived from RNA). Techniques for RNA sequencing also include direct RNA sequencing technologies offered by Oxford Nanopore Technologies and IsoSeq technologies offered by Pacific Biosciences.

Any suitable amplification method may be used, e.g. PCR or RT-PCR.

In one embodiment, the method comprises the step of isolating the amplified DNA.

In one embodiment, the method comprises the step of isolating tagged DNA.

In one embodiment, the method comprises the step of isolating the amplified cDNA.

In one embodiment, the method comprises the step of isolating tagged cDNA.

In one embodiment, the method comprises the step of isolating the amplified DNA and the amplified cDNA.

In one embodiment, the method comprises the step of isolating tagged DNA and tagged RNA.

Suitably, the DNA and/or RNA may be isolated using methods known in the art. For example, the DNA and/or RNA may be isolated using hybridisation-based capturing or magnetic beads.

The sample comprising genomic DNA (e.g. chromatin) may be, for example, a sample of isolated cells, tissue, or whole organs (or other cell-containing biological samples). Suitably, the genomic DNA comprises heterochromatin and euchromatin. Suitably, the sample may comprise genomic DNA which has been extracted from isolated cells, tissue, or whole organs (or other cell-containing biological samples) and optionally fragmented. The sample comprising genomic DNA (e.g. chromatin) may be a sample of permeabilized cells. Preferably, the sample comprising genomic DNA (e.g. chromatin) is a sample of permeabilized nuclei.

The sample comprising genomic DNA (e.g. chromatin) and RNA may be, for example, a sample of isolated cells, tissue, or whole organs (or other cell-containing biological samples). Suitably, the genomic DNA comprises heterochromatin and euchromatin. Suitably, the sample may comprise genomic DNA and RNA which has been extracted from isolated cells, tissue, or whole organs (or other cell-containing biological samples) and optionally fragmented. Preferably, the sample is a nuclei suspension. The sample comprising genomic DNA (e.g. chromatin) and RNA may be a sample of permeabilized cells. Preferably, the sample comprising genomic DNA (e.g. chromatin) and RNA is a sample of permeabilized nuclei.

The methods of the invention do not require pre-processing of genetic material. Thus, the sample may comprise intact cells.

In one embodiment, the method further comprises the step of inducing tagmentation of the genomic DNA following step b), i.e. following addition of the at least one engineered transposase or at least one engineered transposome complex. Certain transposases, such as Tn5, require a Mg²⁺ cofactor for catalysis of transposition. Thus, tagmentation may be induced by the addition of a cofactor, e.g. Mg²⁺, after addition of the transposase.

The sequencing may be single cell sequence analysis.

Methods for DNA sequencing and for RNA sequencing are known in the art (see, for example, Rondinelli, B. et al. (2015) J. Clin. Invest., 125:4625-4637; Reinius, B. et al. (2014) Genome Res., 24:2033-2040; and Buenrostro, J. D. et al. (2013) Nat. Methods, 10:1213-8) may be improved using the engineered transposase, methods and/or use of the present invention.

Bioinformatic methods for the analysis of sequencing data are known in the art. Example methods are described in the Examples herein, although it will be appreciated that any suitable methods and analysis tools may be applied.

The methods of the invention may be used in combination with other approaches for transcriptomic, genomic and/or epigenomic analysis known in the art (e.g. RNA-seq).

Methods for the simultaneous capture of RNA and of euchromatin and heterochromatin, and for the simultaneous preparation of a DNA sequence library and an RNA sequence library, include those described herein (see Example 11).

Simultaneous profiling of the transcriptome, genome and epigenome from the same sample (e.g. from the same cell) provides several advantages. This combined approach, i.e. multiomics approach, maximises the information obtained from limited samples, permits linking of the gene expression profiles with the chromatin conformation state (e.g. regions of accessible chromatin and of condensed chromatin), and eliminates the need for computational analysis across different datasets. In addition, this approach facilitates insights which cannot be gathered solely from a single omics analysis. For example, RNA sequencing does not provide information on copy number variation or non-coding regions of the genome, whereas the present approach provides this information since gene expression analysis is combined with genomic and epigenomic analysis.

The methods of the invention may be used in other aspects of genomic and/or epigenomic research (e.g. to detect chromosomal rearrangements).

In a further aspect, the present invention provides the use of an engineered transposase as described herein for DNA sequencing.

In a further aspect, the present invention provides the use of an engineered transposome as described herein for DNA sequencing.

In a further aspect, the present invention provides the use of an engineered transposase as described herein for genome and epigenetic sequencing.

In a further aspect, the present invention provides the use of an engineered transposome as described herein for genome and epigenetic sequencing.

In a further aspect, the present invention provides the use of an engineered transposase as described herein and at least one further transposase for DNA sequencing.

In a further aspect, the present invention provides the use of an engineered transposome as described herein and at least one further transposome complex for DNA sequencing.

In a further aspect, the present invention provides the use of an engineered transposase as described herein and at least one further transposase for genome and epigenetic sequencing.

In a further aspect, the present invention provides the use of an engineered transposome as described herein and at least one further transposome complex for genome and epigenetic sequencing.

### Method for Making a DNA Sequence Library or Libraries and Optionally a RNA Sequence Library or Libraries

Accordingly, in a further aspect, the present invention provides a method for making a DNA sequence library or libraries comprising the steps:
a) providing a sample comprising genomic DNA;
b) adding at least one engineered transposase as described herein;
c) optionally amplifying tagged DNA; and
d) optionally isolating the amplified DNA.

In a further aspect, the present invention provides a method for making a DNA sequence library or libraries comprising the steps:
a) providing a sample comprising genomic DNA;
b) adding at least one engineered transposome complex as described herein;
c) optionally amplifying tagged DNA; and
d) optionally isolating the amplified DNA.

In a further aspect, the invention provides a method for making a DNA sequence library or libraries and an RNA sequence library or libraries comprising the steps:
a) providing a sample comprising genomic DNA and RNA;
b) (i) adding at least one engineered transposome complex as described herein; and
   (ii) tagging the RNA;
c) optionally amplifying tagged DNA and/or tagged RNA;
d) optionally isolating the amplified DNA and/or the amplified cDNA; and
e) optionally sequencing tagged DNA, the amplified DNA or the isolated DNA and/or optionally sequencing tagged RNA, the amplified cDNA or the isolated cDNA.

In one embodiment, step b) further comprises adding at least one further transposome complex as described herein. The at least one further transposase and/or at least one further transposome complex may bind a component of euchromatin. A DNA sequence library or library for the analysis of both open and condensed chromatin may be generated using the methods of the invention. Suitably, the at least one engineered transposome complex and the at least one further transposome complex may be added simultaneously or sequentially. Preferably, the at least one engineered transposome complex and the at least one further transposome complex are added sequentially. More preferably, the at least one engineered transposome complex is added following the addition of the at least one further transposome complex.

Accordingly, in a further aspect, the invention provides a method for making a DNA sequence library or libraries comprising the steps:
a) providing a sample comprising genomic DNA;
b) adding at least one engineered transposome complex as described herein and at least one further transposome complex as described herein;
c) optionally amplifying tagged DNA; and
d) optionally isolating the amplified DNA.

In a further aspect, the invention provides a method for making a DNA sequence library or libraries and an RNA sequence library or libraries comprising the steps:
a) providing a sample comprising genomic DNA and RNA;
b) (i) adding at least one engineered transposome complex as described herein and at least one further transposome complex as described herein; and
   (ii) tagging the RNA;
c) optionally amplifying tagged DNA and/or tagged RNA;
d) optionally isolating the amplified DNA and/or the amplified cDNA; and
e) optionally sequencing tagged DNA, the amplified DNA or the isolated DNA and/or optionally sequencing tagged RNA, the amplified cDNA or the isolated cDNA.

Suitably, the RNA sequence library or libraries made by the methods of the invention may be a cDNA library or libraries. The cDNA library or libraries is derived from the RNA sequences within the sample.

In one embodiment, the methods of the invention comprise the step of amplifying tagged DNA.

In one embodiment, the methods of the invention comprise the step of amplifying tagged RNA.

In one embodiment, the methods of the invention comprise the step of amplifying tagged DNA and tagged RNA.

Any suitable amplification method may be used, e.g. PCR or RT-PCR.

In one embodiment, the methods of the invention comprise the steps of amplifying tagged DNA and of isolating the amplified DNA.

In one embodiment, the methods of the invention comprise the step of isolating tagged DNA.

In one embodiment, the method comprises the step of isolating the amplified cDNA.

In one embodiment, the method comprises the step of isolating tagged cDNA.

In one embodiment, the method comprises the step of isolating the amplified DNA and the amplified cDNA.

In one embodiment, the method comprises the step of isolating tagged DNA and tagged RNA.

Suitably, the DNA and RNA may be isolated using methods known in the art. For example, the DNA and RNA may be isolated using magnetic beads.

The sample comprising genomic DNA (e.g. chromatin) may be, for example, a sample of isolated cells, tissue, or whole organs (or other cell-containing biological samples). Suitably, the genomic DNA comprises heterochromatin and euchromatin. Suitably, the sample may comprise genomic DNA which has been extracted from isolated cells, tissue, or whole organs (or other cell-containing biological samples) and optionally fragmented. The sample comprising genomic DNA (e.g. chromatin) may be a sample of permeabilized cells. Preferably, the sample comprising genomic DNA (e.g. chromatin) is a sample of permeabilized nuclei.

The sample comprising genomic DNA (e.g. chromatin) and RNA may be, for example, a sample of isolated cells, tissue, or whole organs (or other cell-containing biological samples). Suitably, the genomic DNA comprises heterochromatin and euchromatin. Suitably, the sample may comprise genomic DNA and RNA which has been extracted from isolated cells, tissue, or whole organs (or other cell-containing biological samples) and optionally fragmented. Preferably, the sample is a nuclei suspension. The sample comprising genomic DNA (e.g. chromatin) and RNA may be a sample of permeabilized cells. Preferably, the sample comprising genomic DNA (e.g. chromatin) and RNA is a sample of permeabilized nuclei.

The methods of the invention do not require pre-processing of genetic material. Thus, the sample may comprise intact cells.

Adding the at least one engineered transposase or the at least one engineered transposome complex in step b) results in tagmentation of the sample comprising genomic DNA.

In one embodiment, the method further comprises the step of inducing tagmentation of the genomic DNA following step b), i.e. following addition of the at least one engineered transposase or at least one engineered transposome complex. Certain transposases may require a divalent cation cofactor for catalysis of transposition, e.g. DDE transposases, such as Tn5, may require a Mg²⁺ cofactor. Thus, tagmentation may be induced by the addition of a cofactor, e.g. Mg²⁺, after addition of the transposase.

As used herein, the terms “tagmentation” and “tagment” are used interchangeably to refer to the fragmentation, i.e. cleavage, and tagging of double-stranded DNA. Suitably, in the context of the present invention, tagmentation is performed by the transposase, i.e. by transposition such that the DNA is tagged with the oligonucleotide as described herein. Hence, the oligonucleotide as described herein (i.e. the oligonucleotide comprising ME and optionally tagging sequences and/or sequencing adaptors) may be inserted into the flanking DNA regions of the polypeptide binding site to generate a compatible library (e.g. an NGS compatible library) for sequencing applications.

The methods of the invention may further comprise the step of sequencing tagged DNA, the amplified DNA or the isolated DNA, as appropriate. The methods of the invention may further comprise the step of sequencing tagged DNA, the amplified DNA or the isolated DNA and of sequencing the tagged RNA, the amplified cDNA or the isolated cDNA or RNA, as appropriate. The sequencing may be single cell sequence analysis.

In one embodiment, the tagging sequence of the at least one engineered transposome complex differs from the tagging sequence of the at least one further transposome complex. Thus, the methods of the invention may be used for multiplexed sequencing applications.

In one embodiment, the signal obtained from the at least one further transposome complex and the at least one engineered transposome complex at a DNA locus may be compared.

Methods for making a DNA sequence library or libraries and a RNA sequence library or libraries are known in the art (see, for example, Rondinelli, B. et al. (2015) J. Clin. Invest., 125:4625-4637; Reinius, B. et al. (2014) Genome Res., 24:2033-2040; and Buenrostro, J. D. et al. (2013) Nat. Methods, 10:1213-8) may be improved using the engineered transposase, methods and/or use of the present invention.

The step of tagging the RNA may be performed prior to, at the same time as or after the step of adding the at least one engineered transposase as described herein. Suitably, the step of tagging the RNA is performed after the step of adding the at least one engineered transposase as described herein.

The step of tagging the RNA may be performed prior to, at the same time as or after the step of adding the at least one engineered transposome complex as described herein. Suitably, the step of tagging the RNA is performed after the step of adding the at least one engineered transposome complex as described herein.

The step of tagging the RNA may be performed prior to, at the same time as or after the step of adding the at least one engineered transposome complex as described herein and at least one further transposome complex as described herein. Suitably, the step of tagging the RNA is performed after the step of adding the at least one engineered transposome complex as described herein and at least one further transposome complex as described herein.

In some embodiments, tagging the RNA is performed using an RNA capture probe as described herein.

In some embodiments, tagging the RNA is performed using a polyA capture probe as described herein.

Methods for tagging RNA are known in the art. Tagging the RNA may be carried out using any suitable method, for example, the method disclosed herein (see Example 11).

In one embodiment, the RNA tagging sequence may be from 1 to 100, from 1 to 50 or from 1 to 20 nucleotides in length.

For sequencing (e.g. NGS or RNA-Seq) applications, the RNA tagging sequence may comprise a sequencing adaptor. Suitably, the sequencing adaptor may be an NGS platform-specific tag or RNA-Seq specific required for sequencing. Preferably, the sequencing adaptor is a sequencing primer.

For multiplexed sequencing applications, the RNA tagging sequence may further comprise a unique tagging sequence (also termed a barcode sequence). Suitably, the barcode sequence uniquely labels the RNA tagging sequence species so that it can be distinguished from other RNA tagging sequence species in the reaction for identification in multiplexed sequencing applications in which multiple RNA tagging sequences are used simultaneously with a single sample. The barcode sequence may be a short nucleotide sequence. Suitably, the barcode sequence may be less than 20, less than 10 or 8 bases in length. Preferably, the barcode sequence is 8 bases in length.

In one embodiment, the RNA tagging sequence comprises a sequencing adaptor (e.g. a sequencing primer site).

In one embodiment, the RNA tagging sequence comprises a barcode sequence.

In one embodiment, the RNA tagging sequence comprises a sequencing adaptor (e.g. a sequencing primer site) and a barcode sequence.

In a further aspect, the present invention provides the use of an engineered transposase as described herein for making a DNA sequence library or libraries.

In a further aspect, the present invention provides the use of an engineered transposome complex as described herein for making a DNA sequence library or libraries.

In a further aspect, the present invention provides the use of an engineered transposase as described herein and at least one further transposase for making a DNA sequence library or libraries.

In a further aspect, the present invention provides the use of an engineered transposome complex as described herein and at least one further transposome complex for making a DNA sequence library or libraries.

### Kit

In a further aspect, the present invention provides a kit comprising:
a) at least one engineered transposase as described herein and at least one further transposase; or
b) at least one engineered transposome complex as described herein and at least one further transposome complex.

The kit may further comprise instructions for use of the kit.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of’ as used herein are synonymous with “including”, “includes” or “containing”, “contains”, and are inclusive or open-ended and do not exclude additional, non-recited members, elements or method steps. The terms “comprising”, “comprises” and “comprised of’ also include the term “consisting of’.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Materials and Methods

### Cell Culture

All established cell lines were purchased from American Type Culture Collection (ATCC), except for HEK293T cell line that was a kind gift from Prof. Luigi Naldini (San Raffaele Telethon Institute for Gene Therapy, Milan). Cells were cultured in DMEM (NIH-3T3, HeLa, and HEK293T) or RPMI (Caki-1) supplemented with 10% Fetal Bovine Serum (FA30WS1810500, Carlo Erba for HEK293T and 10270-106 Gibco™ for all the other cell lines) and 1% penicillin-streptomycin (ECB3001D, Euroclone).

### TAM-ChIP

TAM-ChIP (Active Motif) was performed following manufacturer's instructions. Briefly, 10,000,000 of Caki-1 cells crosslinked with 38% formaldehyde; fixation was stopped with 0.125 M glycine. Sonication was then performed on Covaris E220 with the following parameters: total time 6 min, 175 Peak Incident Power, 200 cycles per burst. 8 μg of sonicated chromatin were used as input for each experimental condition. No Antibody (No Ab), Ab anti-H3K9me3 (ab8898 Abcam), Ab anti-H3K4me3 (07-473 Millipore). ChIP-seq, performed as already described in Rondinelli, B. et al. (Rondinelli, B. et al. (2015) J. Clin. Invest., 125:4625-4637), were used as reference for TAM-ChIP-seq (Ab anti-H3K9me3 (ab8898 Abcam) and Ab anti-H3K4me3 (07-473 Millipore) have been used).

### TAM-ChIP-qPCR

TAM-ChIP was performed on two biological replicates for each condition (H3K4me3, H3K9me3 and NoAb). For each biological replicate three technical replicates were analyzed in Real-Time qPCR. In TAMChIP-qPCR one of the two H3K4me3 biological replicates was excluded because no significant signal was detected for any condition. For each TAM-ChIP condition, 10 ng of final libraries were used as input. Water was used as negative control. Real time PCR analysis was performed using Sybr Green Master Mix (Applied Biosystems) on the Viia 7 Real Time PCR System (Applied Biosystems). All primers used were designed on H3K9me3-enriched chromatin regions derived from reference ChIP-seq data (as previously described in Rondinelli, B. et al., supra) and used at a final concentration of 400 nM. To determine the enrichment obtained, we normalized TAM-ChIP-qPCR data for No Ab sample. Primers are listed below in Table 1.

TABLE 1
Primers

| Primer | Forward sequence | Reverse sequence |
|---|---|---|
| FAM5B | GCGCCTTCCTTACTTCCA | AGTGGCCATCTCATTTCCCA |
| | TG | (SEQ ID NO: 36) |
| | (SEQ ID NO: 35) | |
| NTF3 | AAAGGCCTTGGTCCCAGA | ATTGAAGGAACGCAGCCC |
| | (SEQ ID NO: 37) | (SEQ ID NO: 38) |
| CACNA1E | GAGGGAGGAGAAAGCCGA | TTGTCCAGACCAGCCCTT |
| | (SEQ ID NO: 39) | (SEQ ID NO: 40) |

### Tn5 Transposase Production

Tn5 transposase was produced as previously described (Reinius, B. et al. (2014) Genome Res., 24:2033-2040) using pTXB1-Tn5 vector (Addgene, Plasmid #60240). For hybrid transposases, the DNA fragment encoding human HP1a was derived from the pET15b-HP1a (pHP1α-pre) vector (Machida, S. et al. (2018) Mol. Cell, 69:385-397.e8), kindly provided by Dr. Hitoshi Kurumizaka. According to the cloning strategy, two CD (HP1α)-containing regions (spanning residues 1-93 and 1-112) were linked to Tn5, using either a 3 or 5 poly-tyrosine-glycine-serine (TGS) linker, resulting in four hybrid constructs: TnH #1-4 (TnH #1: 93aaCD (HP1α)-3×(TGS)-Tn5; TnH #2: 93aaCD (HP1α)-5×(TGS)-Tn5; TnH #3: 112aaCD (HP1α)-3×(TGS)-Tn5; TnH #4: 112aaCD (HP1α)-5×(TGS)-Tn5. Construct amino acid sequences are detailed below in Table 2.

TABLE 2
Construct amino acid sequences (TGS residues underlined)

| Construct | |
|---|---|
| # | Sequence |
| TnH#1 | MGKKTKRTADSSSSEDEEEYVVEKVLDRRVVKGQVEYLLKWKGFSEEHNTWEPEKN |
| | LDCPELISEFMKKYKKMKEGENNKPREKSESNKRKSNTGSTGSTGSHMITSALHRA |
| | ADWAKSVFSSAALGDPRRTARLVNVAAQLAKYSGKSITISSEGSKAMQEGAYRFIR |
| | NPNVSAEAIRKAGAMQTVKLAQEFPELLAIEDTTSLSYRHQVAEELGKLGSIQDKS |
| | RGWWVHSVLLLEATTFRTVGLLHQEWWMRPDDPADADEKESGKWLAAAATSRLRMG |
| | SMMSNVIAVCDREADIHAYLQDKLAHNERFVVRSKHPRKDVESGLYLYDHLKNQPE |
| | LGGYQISIPQKGVVDKRGKRKNRPARKASLSLRSGRITLKQGNITLNAVLAEEINP |
| | PKGETPLKWLLLTSEPVESLAQALRVIDIYTHRWRIEEFHKAWKTGAGAERQRMEE |
| | PDNLERMVSILSFVAVRLLQLRESFTPPQALRAQGLLKEAEHVESQSAETVLTPDE |
| | CQLLGYLDKGKRKRKEKAGSLQWAYMAIARLGGFMDSKRTGIASWGALWEGWEALQ |
| | SKLDGFLAAKDLMAQGIKIC (SEQ ID NO: 3) |
| TnH#2 | MGKKTKRTADSSSSEDEEEYVVEKVLDRRVVKGQVEYLLKWKGFSEEHNTWEPEKN |
| | LDCPELISEFMKKYKKMKEGENNKPREKSESNKRKSNTGSTGSTGSTGSTGSHMIT |
| | SALHRAADWAKSVFSSAALGDPRRTARLVNVAAQLAKYSGKSITISSEGSKAMQEG |
| | AYRFIRNPNVSAEAIRKAGAMQTVKLAQEFPELLAIEDTTSLSYRHQVAEELGKLG |
| | SIQDKSRGWWVHSVLLLEATTFRTVGLLHQEWWMRPDDPADADEKESGKWLAAAAT |
| | SRLRMGSMMSNVIAVCDREADIHAYLQDKLAHNERFVVRSKHPRKDVESGLYLYDH |
| | LKNQPELGGYQISIPQKGVVDKRGKRKNRPARKASLSLRSGRITLKQGNITLNAVL |
| | AEEINPPKGETPLKWLLLTSEPVESLAQALRVIDIYTHRWRIEEFHKAWKTGAGAE |
| | RQRMEEPDNLERMVSILSFVAVRLLQLRESFTPPQALRAQGLLKEAEHVESQSAET |
| | VLTPDECQLLGYLDKGKRKRKEKAGSLQWAYMAIARLGGFMDSKRTGIASWGALWE |
| | GWEALQSKLDGFLAAKDLMAQGIKIC (SEQ ID NO: 5) |
| TnH#3 | MGKKTKRTADSSSSEDEEEYVVEKVLDRRVVKGQVEYLLKWKGFSEEHNTWEPEKN |
| | LDCPELISEFMKKYKKMKEGENNKPREKSESNKRKSNFSNSADDIKSKKKREQSND |
| | TGSTGSTGSHMITSALHRAADWAKSVFSSAALGDPRRTARLVNVAAQLAKYSGKSI |
| | TISSEGSKAMQEGAYRFIRNPNVSAEAIRKAGAMQTVKLAQEFPELLAIEDTTSLS |
| | YRHQVAEELGKLGSIQDKSRGWWVHSVLLLEATTFRTVGLLHQEWWMRPDDPADAD |
| | EKESGKWLAAAATSRLRMGSMMSNVIAVCDREADIHAYLQDKLAHNERFVVRSKHP |
| | RKDVESGLYLYDHLKNQPELGGYQISIPQKGVVDKRGKRKNRPARKASLSLRSGRI |
| | TLKQGNITLNAVLAEEINPPKGETPLKWLLLTSEPVESLAQALRVIDIYTHRWRIE |
| | EFHKAWKTGAGAERQRMEEPDNLERMVSILSFVAVRLLQLRESFTPPQALRAQGLL |
| | KEAEHVESQSAETVLTPDECQLLGYLDKGKRKRKEKAGSLQWAYMAIARLGGFMDS |
| | KRTGIASWGALWEGWEALQSKLDGFLAAKDLMAQGIKIC (SEQ ID NO: 1) |
| TnH#4 | MGKKTKRTADSSSSEDEEEYVVEKVLDRRVVKGQVEYLLKWKGFSEEHNTWEPEKN |
| | LDCPELISEFMKKYKKMKEGENNKPREKSESNKRKSNFSNSADDIKSKKKREQSND |
| | TGSTGSTGSTGSTGSHMITSALHRAADWAKSVFSSAALGDPRRTARLVNVAAQLAK |
| | YSGKSITISSEGSKAMQEGAYRFIRNPNVSAEAIRKAGAMQTVKLAQEFPELLAIE |
| | DTTSLSYRHQVAEELGKLGSIQDKSRGWWVHSVLLLEATTFRTVGLLHQEWWMRPD |
| | DPADADEKESGKWLAAAATSRLRMGSMMSNVIAVCDREADIHAYLQDKLAHNERFV |
| | VRSKHPRKDVESGLYLYDHLKNQPELGGYQISIPQKGVVDKRGKRKNRPARKASLS |
| | LRSGRITLKQGNITLNAVLAEEINPPKGETPLKWLLLTSEPVESLAQALRVIDIYT |
| | HRWRIEEFHKAWKTGAGAERQRMEEPDNLERMVSILSEVAVRLLQLRESFTPPQAL |
| | RAQGLLKEAEHVESQSAETVLTPDECQLLGYLDKGKRKRKEKAGSLQWAYMAIARL |
| | GGFMDSKRTGIASWGALWEGWEALQSKLDGFLAAKDLMAQGIKIC |
| | (SEQ ID NO: 7) |

### Transposon Assembly

Assembly of standard and modified pre-annealed Mosaic End Double-Stranded (MEDS) oligonucleotides, Tn5MEDS-A, Tn5MEDS-B, Tn5MEDS-A and TnHMEDS-A was performed in solution following published protocol (Reznikoff, W. S. (2008) Annu. Rev. Genet., 42:269-286). For single cell GET-seq, standard ME-A oligo49 was replaced by a combination of eight different sequences containing 8 nt tags before the 19 nt ME sequence to allow differentiation of fragments derived from either Tn5 or TnH tagmentation. Four sequences were used to replace standard Tn5ME-A (Tn5ME-A. 1, Tn5ME-A.2, Tn5ME-A.7, Tn5ME-A.8) and other four sequences for TnHME-A (TnHME-A.4, TnHME-A.5, TnHME-A.9, TnHME-A. 10). A Read 1 primer binding site was reconstituted adding 8 nt (TCCGATCT) upstream the Tn5/TnH tag. Modified Tn5ME-A sequences are detailed below in Table 3.

TABLE 3
Modified Tn5ME-A sequences (Tn or TnH associated barcode underlined):

| Tn5/HME-A. X | Sequence |
|---|---|
| Tn5ME-A. 1 | 5′-TCGTCGGCAGCGTCTCCGATCTCGTACTAGAGATGTGTATAAGAGACAG-3′ |
| | (SEQ ID NO: 27) |
| Tn5ME-A. 2 | 5′-TCGTCGGCAGCGTCTCCGATCTTCCTGAGCAGATGTGTATAAGAGACAG-3′ |
| | (SEQ ID NO: 28) |
| Tn5ME-A. 7 | 5′-TCGTCGGCAGCGTCTCCGATCTTCATGAGCAGATGTGTATAAGAGACAG-3′ |
| | (SEQ ID NO: 29) |
| Tn5ME-A. 8 | 5′-TCGTCGGCAGCGTCTCCGATCTCCTGAGATAGATGTGTATAAGAGACAG-3′ |
| | (SEQ ID NO: 30) |
| TnHME-A. 4 | 5′-TCGTCGGCAGCGTCTCCGATCTTAAGGCGAAGATGTGTATAAGAGACAG-3′ |
| | (SEQ ID NO: 31) |
| TnHME-A. 5 | 5′-TCGTCGGCAGCGTCTCCGATCTGCTACGCTAGATGTGTATAAGAGACAG-3′ |
| | (SEQ ID NO: 32) |
| TnHME-A. 9 | 5′-TCGTCGGCAGCGTCTCCGATCTAGGCTCCGAGATGTGTATAAGAGACAG-3′ |
| | (SEQ ID NO: 33) |
| TnHME-A.10 | 5′-TCGTCGGCAGCGTCTCCGATCTCTGCGCATAGATGTGTATAAGAGACAG-3′ |
| | (SEQ ID NO: 34) |

Creation of functional transposon was performed following previously published protocol (Reinius, B. et al. (2014) Genome Res., 24:2033-2040).

### Bulk Tagmentation Reaction and ATAC-Seq

Bulk tagmentation was performed on Caki-1 genomic DNA (gDNA) following published protocol (Reinius, B. et al. (2014) Genome Res., 24:2033-2040). Specifically, 500 ng of gDNA was incubated for 7 min at 55° C. with 1 μL of functional transposon in 1×TAPS-PEG8000 buffer in a final 20 μL volume. As control, a parallel reaction was carried out on Caki-1 gDNA but using the Nextera DNA Library Prep Kit according to the manufacturer's protocol. Reactions were stopped adding SDS at a final concentration of 0.05% and incubated for 5 min at room temperature (RT). Then 5 L of this mixture was used as input for indexing PCR using standard Nextera N7xx and S5xx oligos and KAPA HiFi enzyme (Roche) using the following protocol: 3 min at 72° C., 30 sec at 98° C. followed by 13 cycles of 45 sec at 98° C., 30 sec at 55° C., 30 sec at 72° C. . . . Libraries were then purified using 1× volume of Ampure XP beads (Beckman-Coulter) and checked for fragment distribution on TapeStation (Agilent).

ATAC-seq was performed following published protocols (Buenrostro, J. D. et al. (2013) Nat. Methods, 10:1213-8) with minor modifications.

Briefly, 100,000 Caki-1 cells pellets were washed in 100 μL cold 1×PBS, centrifuged for 10 min at 500*g at 4° C., and permeabilized in 100 μL of cold lysis buffer (10 mM Tris·Cl, pH 7.4, 10 mM NaCl, 3 mM MgCl2, 0.1% (v/v) Igepal CA-630), then centrifuged again for 10 min at 500*g at 4° C. Tagmentation was performed on cell pellets-using either Tn5 or TnH—by adding 100 μL of transposition mix (5×TAPSPEG8000 buffer mixed with 10 μL of 1.39 UM of functional transposon in a final volume of 100 μL). As control, a parallel reaction was carried out on 100,000 Caki-1 cells pellets using the Nextera XT DNA Library Prep Kit according to the manufacturer's protocol. Reactions were performed at 37° C. for 30 min and stopped adding SDS at a final concentration of 0.05%. After 5 min of incubation at RT, reactions were purified using QIAquick Gel Extraction Kit (Qiagen) and eluted in 15 μL of EB buffer. 5 μL of this reaction was used as input for indexing PCR as described before.

Libraries were sequenced on Illumina platforms with 2×50 bp sequencing protocol.

### Single Cell ATAC-Seq and GET-Seq

Single-cell ATAC-seq was performed on Chromium platform (10× Genomics) using “Chromium Single Cell ATAC Reagent Kit” V1 Chemistry (manual version CG000168 Rev C), and “Nuclei Isolation for Single Cell ATAC Sequencing” (manual version CG000169 Rev B) protocols. Nuclei suspension was prepared in order to get 10,000 nuclei as target nuclei recovery.

Single cell GET-seq was performed as previously described but replacing the provided ATAC transposition enzyme (10× Tn5; 10× Genomics) with a combination of Tn5 and TnH functional transposons, in the transposition mix assembly step. Specifically, a sequential Tn5 to TnH reaction was performed: a transposition mix contained 1.5 μL of 1.39 UM Tn5 was incubated for 30 min at 37° C., then 1.5 μL of 1.39 μM TnH was added and the reaction was continued for a total of 1 h incubation.

When scGET-seq was performed on 20:80 proportion of HeLa: Caki-1 cells, nuclei suspension was prepared in duplicate in order to get 10,000 nuclei as target nuclei recovery for each replicate.

Final libraries were loaded on Novaseq6000 platform (Illumina) to obtain 50,000 reads/nucleus with 2×50 bp read length. For GET-seq, the sequencing target was 100,000 reads/nucleus; and a custom Read 1 primer (0.5 μM final concentration) was added to the standard Illumina mixture (5′-TCGTCGGCAGCGTCTCCGATCT-3′; SEQ ID NO: 41).

### Single Cell RNA-Seq

Single-cell RNA-seq was performed on Chromium platform (10× Genomics) using “Chromium Single Cell 3′ Reagent Kits v3” kit manual version CG000183 Rev C (10× Genomics). Final libraries were loaded on Novaseq6000 platform (Illumina) to obtain 50,000 reads/cells.

### Kdm5c Knock-Down Experiment

Lentiviral vectors were produced by transfecting HEK293T cells (a kind gift from Prof. Luigi Naldini, San Raffaele Telethon Institute for Gene Therapy, Milan) with pLK0.1 plasmid containing shRNAs targeting Kdm5c (shKdm5c, CCGGGCAGTGTAACACACGTCCATTCTCGAGAATGGACGTGTGTTACACTGCTTTT; SEQ ID NO: 42) or scramble (shScr; Rondinelli, B. et al. (2015) J. Clin. Invest., 125:4625-4637).

Calcium chloride method was used for transfection. Specifically, a mix containing 30 μg of transfer vector, 12.5 μg of Δr 8.74, 9 μg of Env VSV-G, 6.25 μg of REV, 15 μg of ADV plasmid, was prepared and filled up to 1125 μl with 0.1× TE/dH2O (2:1); after 30 min of incubation on rotation, 125 μl of 2.5 M CaCl2 were added to the mix and, after 15 min of incubation, the precipitate was formed by dropwise addition of 1,250 μl of 2×HBS to the mix while vortexing at full speed; finally 2.5 ml of precipitate was added drop by drop to 15 cm dishes with HEK293T cells at 50% confluency. After 12-14 h the medium was replaced with 16 ml fresh medium/dish supplemented with 16 μl of NAB/dish. After 30 h the medium containing viral particles was collected, filtered with 0.22 μm filter and stored at −80° C. in small aliquots to avoid freeze-thaw cycles.

NIH-3T3 cells were transduced in 6 well-plate format. To this end, 2 ml of shKdm5c/shScr lentiviral vector supplemented with Polibrene (final concentration 8 μg/ml) were added to actively cycling (50% confluency) NIH-3T3; one well of untransduced cells was used as negative control. After 24 h transduced cells were splitted in a 10 cm dish and Puromycin selection (final concentration 4 μg/ml) was performed. 48 h post selection half of transduced cells were detached, washed twice with cold 1×PBS and tested for gene knockdown by Real Time (RT)-PCR as described below. Upon validation of knock-down, 72 h post selection, all the remaining cells were collected and subjected to scGET-seq as already described. Nuclei suspension was prepared in order to get 10,000 nuclei as target nuclei recovery.

### Gene Knock-Down Validation by Real Time (RT)-qPCR

Total RNA was isolated using Trizol (Invitrogen, Carlsbad, CA, USA) and purified using RNeasy mini kit (Qiagen); cDNA was generated using First-Strand cDNA Synthesis ImpromII A3800 kit (Promega), with random primers. RT-qPCR was performed using Sybr Green Master Mix (Applied Biosystems) on the Viia 7 Real Time PCR System (Applied Biosystems). 10 ng of cDNA were used as input, water was used as negative control. Amplification was performed using previously validated primers (Rondinelli, B. et al. (2015) J. Clin. Invest., 125:4625-4637) and used at a final concentration of 400 nM except for major that were used 200 nM. Primers for minor ncRNA were taken from Zhu, Q. et al. (Zhu, Q. et al. (2011) Nature, 477:179-184) and were used at a final concentration of 400 nM.

Fibroblast Reprogramming Towards iPSC and iPSC Differentiation Towards NPC

Dermal fibroblasts (FIB) obtained from skin biopsies of two different healthy subjects (A and B) were cultured in fibroblast medium and reprogrammed with the Sendai virus technology (CytoTune-iPS Sendai Reprogramming Kit, ThermoFisher, Waltham, MA, USA) to generate Human induced pluripotent Stem Cells (iPSC) clones. iPSC clones were individually picked, expanded and maintained in mTeSR1 on hESCqualified Matrigel. Human iPSC-derived neural progenitor cells (NPC) were generated following the standard protocol based on a dual-smad inhibition (Reinhardt, P. et al. (2013) PLOS One, 8: e59252). Briefly, iPSCs were differentiated in NPC via human embryoid bodies. Neural induction was initiated through inhibition using the dual-small inhibition molecules dorsomorphin, purmorphamine, and SB43152. The small molecule CHIR99021, a GSK3b inhibitor, was added to stimulate the canonical WNT signalling pathway. The study was approved by Comitato Etico Ospedale San Raffaele (BANCA-INSPE Sep. 3, 2017).

Human FIB, iPSC and NPC derived from patient A and B were collected, counted and subjected to GETseq as already described. Nuclei suspension was prepared in order to get 5,000 nuclei as target nuclei recovery.

### Patient-Derived Colorectal Cancer Organoids (PDOs)

Samples from 2 patients were obtained upon written informed consent. This study was carried out in accordance with protocols approved by the San Raffaele Hospital Istitutional Review Board, and the procedures followed were in accordance with the Declaration of Helsinki of 1975, as revised in 2000.

Establishment and culture of PDOs were performed as previously reported (Vlachogiannis, G. et al. (2018) Science, 359:920-926). Briefly, fresh tumor specimens obtained from patients with liver metastatic gastrointestinal cancers were used immediately after surgery. Tissues were minced, conditioned in PBS/5 mM EDTA and digested in a solution composed of PBS/1 mM EDTA, 2× TrypLE™ Select Enzyme (Thermofisher) and DNAse I (Merck) for 1 h at 37° C. Release of the cells from the tissue was facilitated by pipetting. Dissociated cells were collected, resuspended in 120 μl growth factor reduced (GFR) Matrigel™ (Corning™ 356231, FisherScientific), seeded in single domes in 24-well flat bottom cell culture plate (Corning) and, after dome solidification, overlaid with 1 ml of complete human organoid medium (Vlachogiannis, G. et al. (2018) Science, 359:920-926) and medium replaced every two/three days. PDOs were dissociated to single cells either for passaging after reaching confluence or for the subsequent downstream applications by mechanical and enzymatic digestion. PDOs were retrieved from Matrigel™ in a solution composed of PBS/1 mM EDTA and 1× TrypLE™ Select Enzyme (Thermofisher), incubated for 20 min at 37° C. then dissociated to single cells by pipetting. Cells were harvested, resuspended in growth factor reduced (GFR) Matrigel™ (Corning™ 356231, FisherScientific), and seeded at an appropriate ratio. Alternatively, 100.000 cells were suspended in 15 μl nucleic buffer.

### Patient-Derived Colorectal Cancer Xenografts

Specimen collection and annotation-EGFR blockade responsive colorectal cancer and matched normal samples were obtained from one patient that underwent liver metastasectomy at the Azienda Ospedaliera Mauriziano Umberto I (Torino). The patient provided informed consent. Samples were procured and the study was conducted under the approval of the Review Boards of the Institution.

PDX models and in vivo treatment-Tumour implantation and expansion were performed in 6-week-old male and female NOD (nonobese diabetic)/SCID (severe combined immunodeficient) mice as previously described (Bertotti, A. et al. (2011) Cancer Discov., 1:508-523). Once tumours reached an average volume of ˜400 mm3, mice were randomized into treatment arms that received either placebo or cetuximab (Merck, 20 mg/kg twice weekly, intraperitoneally) as follows: untreated n=1; 72 hours cetuximab treatment n=2; 4 weeks cetuximab treatment n=4; 7 weeks cetuximab treatment n=5. Each of the treatment arms was replicated twice. In order to reach the endpoint of all the experimental groups on the same day, treatments were started asynchronously. Tumour growth was monitored once weekly by caliper measurements, and approximate tumour volumes were calculated using the formula 4/3p. (d/2) 2. D/2, where d and D are the minor tumour axis and the major tumour axis, respectively. Operators were blinded during measurements. In vivo procedures and related biobanking data were managed using the Laboratory Assistant Suite (DOI 10.1007/s10916-012-9891-6). Animal procedures were approved by the Italian Ministry of Health (authorization 806/2016-PR).

Single cell GET-seq on PDXA—At the end of treatments, mice were sacrificed and tumors collected. All the tumours pertaining to each treatment arm were pooled together and minced through mechanical procedure with sterile scalpels. The dissociation step was performed through mechanical and enzymatic means using the Human Tumor Dissociation Kit (Miltenyi Biotec) in disposable gentleMACS™ C Tubes (Miltenyi Biotech) with the gentleMACS™ Dissociator (Miltenyi Biotec) according to the manufacturer's protocol. The suspensions were then filtered through a 100 UM and a 40 μM cell strainer (Corning Life Sciences). The number of recovered viable cells was evaluated with the automated cell counter Countess (Invitrogen) coupled with Trypan Blue staining. Single cells were then subjected to single-cell GET-seq as already described. Nuclei suspension was prepared in order to get 10,000 nuclei as target nuclei recovery for each replicate.

### Bioinformatics Analysis

### Data Preprocessing

Illumina sequencing data for bulk sequencing were demultiplexed using bcl2fastq using default parameters. Sequencing data for single cell experiments were demultiplexed using cellranger-atac (v1.0.1). Identification of cell barcodes was performed using umitools (v1.0.1; Smith, T. et al. (2017) Genome Res., 27:491-499) using R2 as input.

Read tags for GET-seq and scGET-seq experiments, where TnH and Tn5 data are mixed, were processed with tagdust (v2.33; Lassmann, T. (2015) BMC Bioinformatics, 16:1-8) specifying transposase-specific barcodes as first block in the HMM model.

Bulk data are processed using the following code:

tagdust −1 \
B:TAAGGCGA, GCTACGCT, AGGCTCCG, CTGCGCAT,
CGTACTAG, TCCTGAGC, TCATGAGC, CCTGAGAT \
−2 R:N \
${sample}_R[12]*fastq.gz \
−o $ {sample}

Single cell data were processed using the following code:

tagdust −1 \
B:TAAGGCGA, GCTACGCT, AGGCTCCG, CTGCGCAT,
CGTACTAG, TCCTGAGC, TCATGAGC, CCTGAGAT \
−2 S:AGATATATATAAGGAGACAG −3 R:N \
${sample}_R[123]*fastq.gz ${sample}_I1_*.fastq.gz \
−o ${sample}

fastq files were then merged according to the barcode sets (TnH: TAAGGCGA, GCTACGCT, AGGCTCCG, CTGCGCAT; Tn5: CGTACTAG, TCCTGAGC, TCATGAGC, CCTGAGAT). Reads for ChIP-seq, GET-seq, scGET-seq experiments were aligned to reference genome (hg38 or mm10) using bwa mem v0.7.12 (Li, H. (2013) arXiv, 00:1-3).

### Analysis of Bulk Sequencing Data

Aligned reads were deduplicated using samblaster (Faust, G. G. & Hall, I. M. (2014) Bioinformatics, 30:2503-2505). Genome bigwig tracks were generated using bamCoverage from the deepTools suite (Ramirez, F. et al. (2014) Nucleic Acids Res., 42:187-191). H3K4me3 enriched regions were identified using MACS v2.2.7 (Zhang, Y. et al. (2008) Genome Biol., 9: R137). H3K9me3 enriched regions were identified using SICER v2 (Anders, S. (2009) Bioinformatics, 25:1231-1235), using default parameters. Hilbert curves were generated using hc_bigwig.py script from gilbert (https://bitbucket.org/dawe/gilbert), a reimplementation of HilbertVis al. (Breeze, C. E. et (2020) bioRxiv doi: 10.1101/2020.06.26.172718), using level 8 summarization and log-scale plotting. Overlay of Hilbert curves was obtained using ImageJ (Schneider, C. A. et al. (2012) Nat. Methods, 9:671-675).

### Definition of Epigenome Reference Sets

In order to analyze single cell data with joint information of accessible and compacted chromatin, we segmented the genome according to DNAsel Hypersensitive Sites (DHS), as previously described (Giansanti, V. et al. (2020) F1000Research, 9:199).

Briefly, we downloaded the index of DHS for human (Meuleman, W. et al. (2020) Nature, 584:244-251.) and mouse genome (Breeze, C. E. et al. (2020) bioRxiv doi: 10.1101/2020.06.26.172718), intervals closer than 500 bp were merged using bedtools (Quinlan, A. R. (2014) Current Protocols in Bioinformatics doi: 10.1002/0471250953.bi1112s47) to create the interval set for accessible chromatin (named “DHS”). We then took the complement of the set to create the interval set for compacted chromatin (named “complement”).

Analysis of scGET-Seq Data

Lists of accepted cellular barcodes were assigned to reads inside aligned BAM files using bc2rg.py script from scatACC (https://github.com/dawe/scatACC), duplicated reads were then identified at cell level using cbdedup.py script from the same repository. For each scGET-seq experiment we generated four count matrices: Tn5-dhs, Tn5-complement, Tnh-dhs and TnH-complement, profiling Tn5 and TnH over accessible and compacted chromatin respectively. Count matrices were generated using peak_count.py script from scatACC repository. Each count matrix was processed using scanpy v1.4.6 (Wolf, F. A et al. (2018) Genome Biol., 19:1-5); after an initial filtering on shared regions and number of detected regions per cell, matrices were normalized and log-transformed. The number of regions was used as covariate for linear regression and data were then scaled with a maximum value set to 10. Neighbourhood was evaluated using Batch balanced KNN (Polański, K. et al. (2020) Bioinformatics, 36:964-965), cell groups were identified with Leiden algorithm (Traag, V. A. et al. (2019) Sci. Rep., 9:1-12) for cell lines or schist (Morelli, L. et al. (2020) bioRxiv. doi: 10.1101/2020.06.28.176180) choosing the hierarchy level that maximizes modularity. In order to extract a unique representation of four datasets, we applied graph fusion using scikit-fusion (Žitnik, M. & Zupan, B. (2015) IEEE Trans. Pattern Anal. Mach. Intell., 37:41-53): we first extracted a 20-components UMAP reduction of each view, then we built a relation graph where all views are connected to a 20-components Latent Space (LS). Matrix factorization was run with 1000 iterations 5 times. The resulting LS was then added in each scanpy object as the basis for neighbourhood evaluation and cell clustering.

### Library Saturation Estimates

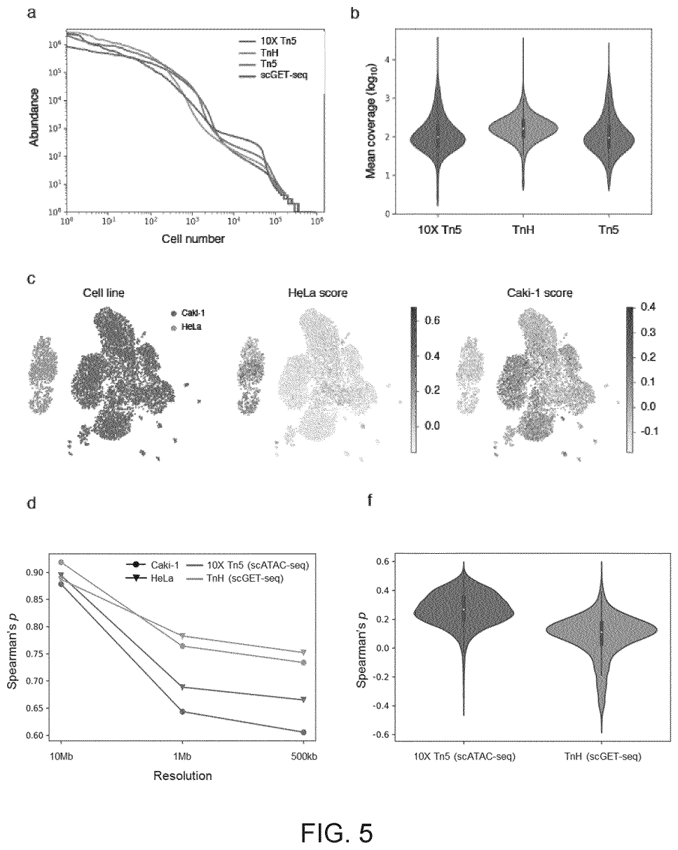

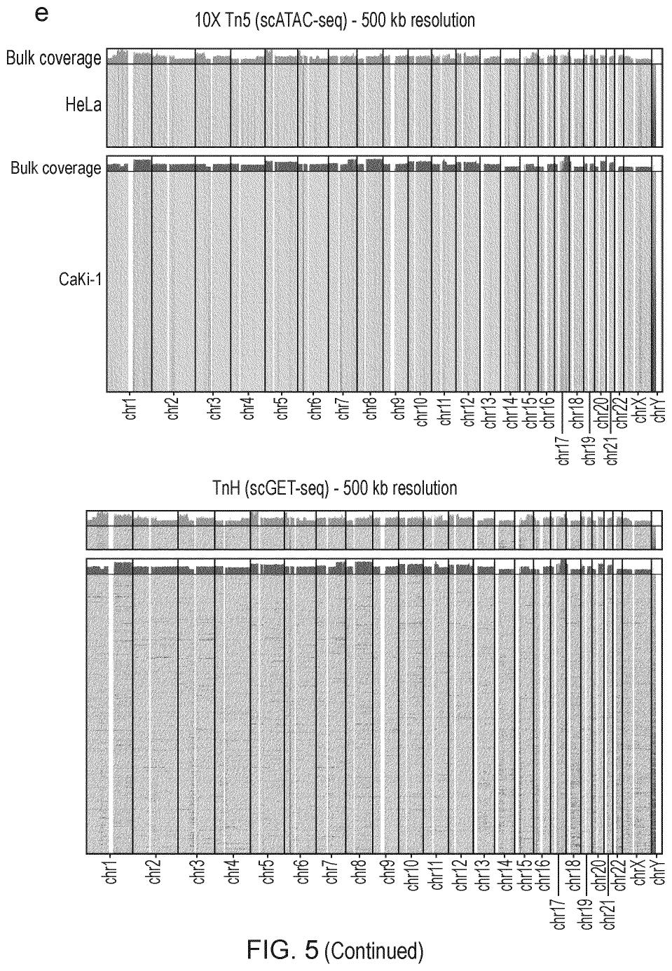

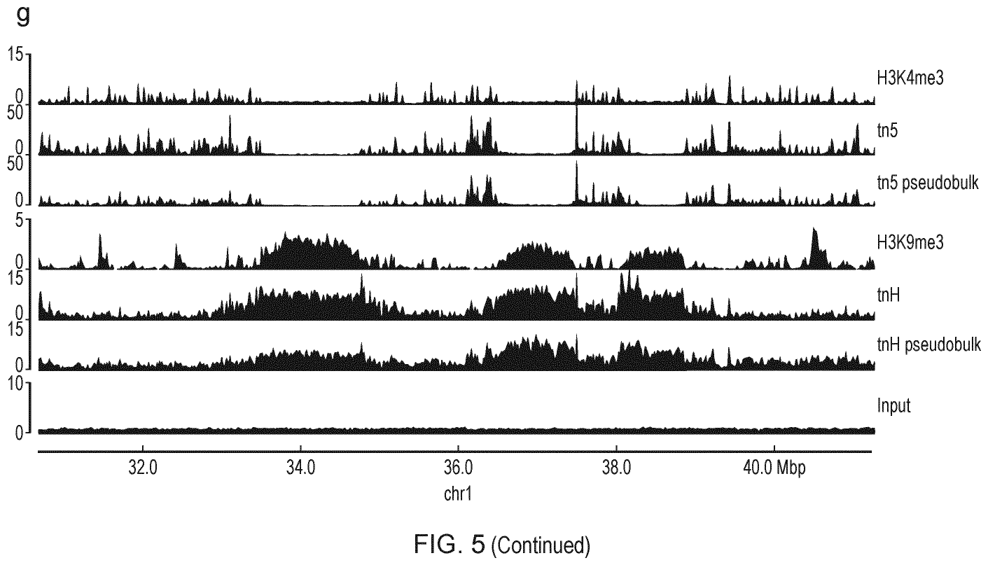

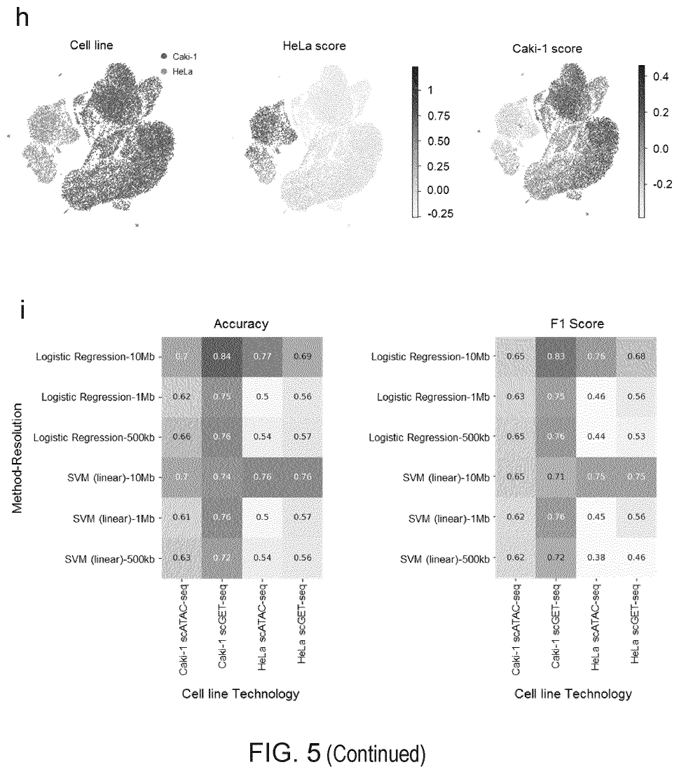

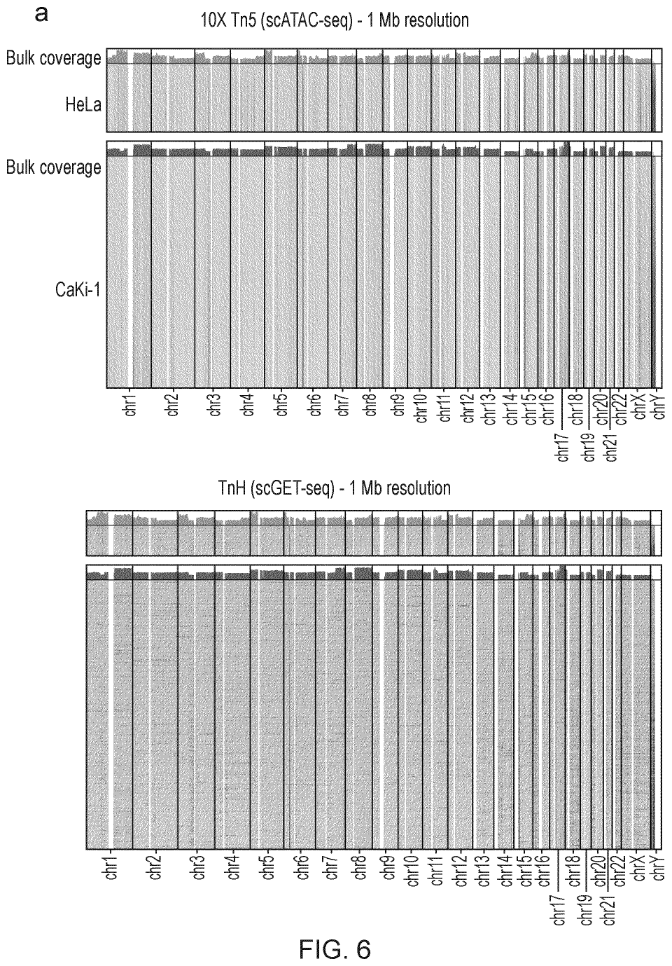

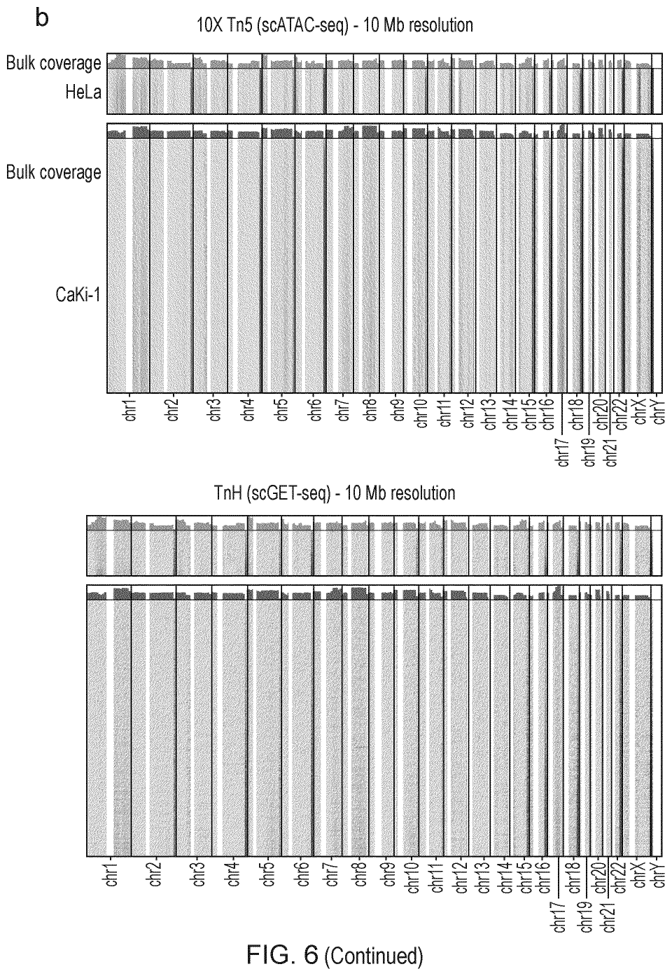

To estimate the library complexity we first downsampled 10 datasets (4 depicted in FIG. 5a and 6 randomly chosen) at different proportions (0.1×, 0.2×, 0.5×) and calculated the number of genomic bins (5 kb) that could be found in each dataset. For each dataset we fitted the shape parameter s of a lower incomplete Gamma function. We then built a linear model fitting the number of cells and the number of duplicates to predict s. We obtained the model s=0.815·N_{cells}+0.406. (1-d)+0.2316, where N_{cells} is the number of cells divided by 1000 and d is the fraction of duplicated reads.

### Analysis of HeLa/Caki-1 Cell Identity

In order to identify cell identity in Caki-1/HeLa mixture, we downloaded publicly available bulk ATACseq for Hela cells (GSE106145; Cho, S. W. et al. (2018) Cell, 173:1398-1412.e22) and preprocessed as described above. We then generated a count matrix for Hela cells and our bulk ATAC-seq for Caki-1 cells over the DHS regions, using bedtools.

The resulting matrix was analyzed using edgeR (Robinson, M. D et al. (2009) Bioinformatics, 26:139-140) using RLE normalization and contrasting HeLa vs Caki by exact test. We selected HeLa specific regions by filtering for FDR <1e-3, log CPM>3 and log FC>0 (i.e. regions enriched in Hela cells, with detectable read counts), and we took the top 200 regions that were present in scGET-seq data. We used this list to create a HeLa score using the score_genes function implemented in scanpy.

### Cell Cycle Analysis

Identification of cell cycle phase using replication data was performed as follows. First, we identified high-coverage and low-coverage cells in each experiment, by analyzing TnH-complement data, we then identified the top 500 Tn5-dhs regions characterizing each cluster.

2-stage Repli-seq data for NIH-3T3 cells were downloaded from the 4DNucleome project

(https://data.4dnucleome.org/experiment-set-replicates/4DNES7ZVDD5G/), replicated data were averaged and the log 2-ration between early stage (E) and late stage (L) was calculated. Entries in Tn5-dhs list were assigned the average log 2 (E/L) value over the its interval.

LaminB1 DamID data for NIH-3T3 cells were also downloaded from UCSC genome browser tables, converted to bigwig format and lifted over mm10 assembly coordinates using Crossmap (Zhao, H. et al. (2014) Bioinformatics, 30:1006-1007). Average value of LaminB1 data over Tn5-dhs regions was assigned as described above.

Differences in distribution of log 2 (E/L) and LaminB1 values were evaluated by Mann-Whitney U test.

### Analysis of Copy Number Alterations

Copy Number Alteration were derived from TnH data counted over the entire genome, binned at 5 kbp resolution. Counts were extracted using peak_count.py script from the scatACC repository.

After that, data were processed by collapsing values into larger bins at different resolutions (10 Mb, 1 Mb, 500 kb). The value of each bin is divided by the average per-cell read count; we apply linear regression of per bin GC content and mappability (Karimzadeh, M. et al., (2018) Nucleic Acids Res., 46: e120), retrieved from UCSC genome browser, and finally express values as log 2 of the scaled residuals. Cell clustering was performed using schist applied on the kNN graph built with bbknn and using correlation as distance metric. The number of clusters is defined by the highest level of the hierarchy that splits more than one group. Evaluation of the posterior distribution of number of groups is performed by equilibration of a Markov Chain Monte Carlo model with at most 1,000,000 iterations.

### Classification of CNA in Caki-1: Hela Cells

We created a ground truth dataset by calling copy number alterations in Caki-1 and Hela cells with Control-FREEC (Karimzadeh, M. et al., (2018) Nucleic Acids Res., 46: e120) on Whole Genome Sequencing data. We binned the resulting segments according to the desired resolution in single cell experiments (10 Mb, 1 Mb and 500 kb), retaining three classes (loss, gain and normal).

We subsampled scATAC-seq cells and scGET-seq cells to match cell numbers and coverage distributions, to avoid biases due to different data sizes. We split log 2 ratio matrices into a training and a test set in 70:30 proportion. We trained a Logistic Regression classifier and a Support Vector Machine with the one vs-rest strategy and increasing the number of iterations to ensure convergence. We recorded accuracy and F1-score on the test sets. This process was applied on each resolution, cell type and platform.

### Bulk Analysis of Organoids Whole Exome Sequencing Data

Reads were aligned to hg38 reference genome using bwa, reads were then processed using bwa. Alignment were processed using GATK MarkDuplicates and Base Quality Score Recalibration (Karimzadeh, M. et al., (2018) Nucleic Acids Res., 46: e120). Somatic mutations and copy number segments were identified with Sequenza (Favero, F. et al. (2015) Ann. Oncol. Off. J. Eur. Soc. Med. Oncol., 26:64-70) with default parameters. Evaluation of CNV was performed using CNAqc (Househam, J. et al. (2021) bioRxiv 2021.02.13.429885), clonal deconvolution was performed using MOBSTER and BMix (Caravagna, G., et al. (2020) BMC Bioinformatics 21:531) with default parameters.

### Analysis of Mutations

Reads for Tn5 and TnH data were separated to individual BAM files using separate_bam.py script from the scatACC repository. Known somatic mutations were genotyped using freebayes v. 1.3.2 (Garrison and Marth).

freebayes −f hg38.fa −1 −@ somatic.vcf.gz −C 2 −F 0.01*, bam

Only variants with depth >1 were then considered for the analysis.

Variant calling without priors was performed using freebayes using the same thresholds. VCF files were annotated using snpEff v4.3p (Cingolani, P. et al. (2012) Fly (Austin)., 6:80-92) using GRCh38.86 annotation model. Known cancer variants were annotated using COSMIC catalog (Forbes, S. A. et al. (2011) Nucleic Acids Res., 39:945-950). Variants were then filtered for depth >10, quality >5 if unknown, and quality >1 if profiled in COSMIC.

### Chromatin Velocity

Chromatin velocity was calculated using scvelo (Bergen, V. et al. (2020) Nat. Biotechnol. doi: 10.1101/820936). Normalized count matrices over DHS regions for Tn5 and TnH were first filtered to include regions common to both. Then a proper object was created injecting Tn5 and TnH data in the unspliced and spliced layers respectively. Moments were calculated using default parameters. Dynamical modelling was then applied and final velocity was calculated using the differential kinetics knowledge. Regions having a likelihood value higher than the 95-percentile were considered as marker regions.

Analysis of scRNA-Seq Data

Reads were demultiplexed using cellranger (v4.0.0). Identification of valid cellular barcodes and UMIs was performed using umitools with default parameters for 10× v3 chemistry. Reads were aligned to hg38 reference genome using STARsolo (v2.7.7a) (Dobin, A. et al. (2013) Bioinformatics, 29:15-21 and/or f1000research. 1117634.1). Quantification of spliced and unspliced reads on genes was performed by STARsolo itself on GENCODE v36 (Harrow, J. et al. (2012) Genome Res., 22:1760-1774). Count matrices were imported into scanpy, doublet rate was estimated using scrublet (Wolock, S. L., et al., (2019) Cell Syst., 8:281-291.e9). Count matrix was filtered (min_genes=200, min_cells=5, pct_mito<20) before normalization and log-transformation. kNN graph was built using bbknn. RNA velocity was estimated using scvelo dynamical modeling with latent time regularization.

### Total Binding Affinity Analysis

For each DHS region selected for likelihood, we extracted the 500 bp sequence flanking summits there included, as annotated in the DHS index. We downloaded the HOCOMOCO v11 list of PWM (Kulakovskiy, I. V. et al. (2018) Nucleic Acids Res., 46: D252-D259) and calculated the Total Binding Affinity as defined in Molineris, I. et al. (Molineris, I. et al. (2011) Mol. Biol. Evol., 28:2173-2183) using tba_nu.py script from the scatACC repository. TBA values for multiple summits within a DHS regions were summed. Final values were divided by the length of the corresponding DHS region. In order to obtain a cell-specific TBA value, the region-by-TBA matrix was multiplied by the cell-by-region velocity matrix.

PLS analysis was performed using PLSCanonical function from the python sklearn.cross_decomposition library, using cell groups as targets for the matrix transformation.

### Example 1-Tn5 is Able to Tagment Compacted Chromatin Featuring H3K9Me3

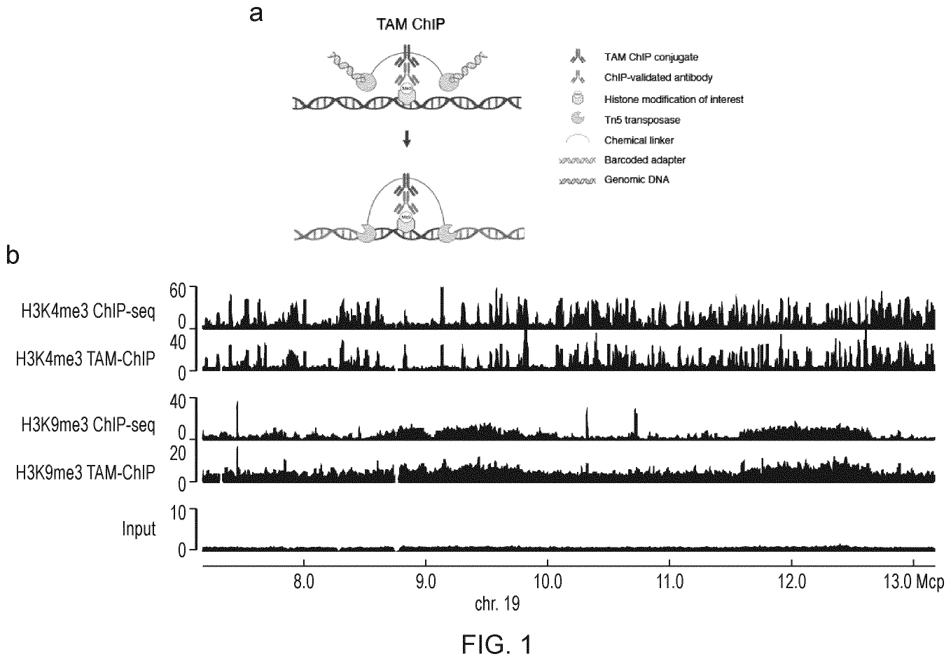

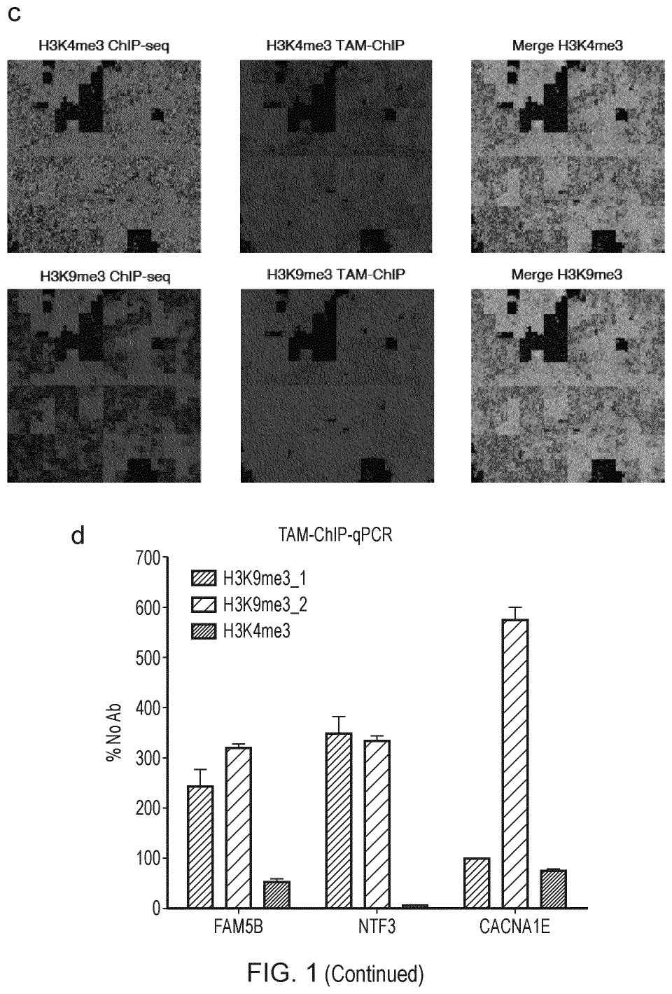

We first determined whether transposase 5 (Tn5), which is commonly used to probe accessible DNA in ATAC-seq, is also able to tagment compacted chromatin, if properly redirected. To this end, we exploited a Transposase-Assisted Chromatin Immuno-Precipitation (TAM-ChIP) approach, which combines the antibody-mediated targeting of chromatin immuneprecipitation with the ability of Tn5 to tagment DNA, leading to chromatin fragmentation and barcoding of the chromatin surrounding the antibody binding site (FIG. 1a).

Because of its relevance, we decided to explore H3K9me3 histone modifications. We choose a primary antibody recognizing the histone mark H3K9me3 (or H3K4me3, as control), which was then bound by a secondary antibody conjugated to Tn5. H3K4me3 TAM-ChIP-seq profiles mirrored the corresponding ChIP-seq profiles obtained with a H3K4me3 antibody. Instead, when conjugated with an antibody targeting H3K9me3, Tn5 tagmented preferentially H3K9me3-enriched, compacted chromatin regions (FIGS. 1b and c). These results were also confirmed by Real Time-qPCR (FIG. 1d).

All together, these experiments demonstrate that Tn5 is able to fragment and tag not only accessible chromatin regions, but if properly redirected, also H3K9me3-compacted chromatin.

### Example 2-Hybrid CD (HP1α)-Tn5 Targets H3K9Me3 Chromatin Regions

TAM-ChIP using Tn5 targeted towards H3K9me3 was only partially effective in redirecting the transposase towards closed chromatin. Additionally, this approach relies on antibodies, which pose technical challenges.

We hence explored targeting compacted chromatin via the modification of the natural tropism of the Tn5 transposase, targeting it directly towards H3K9me3-labeled chromatin. To this end, we selected heterochromatin protein 1-α (HP1α), involved in heterochromatin assembly and maintenance, which specifically binds H3K9me3, through its chromodomain (CD).

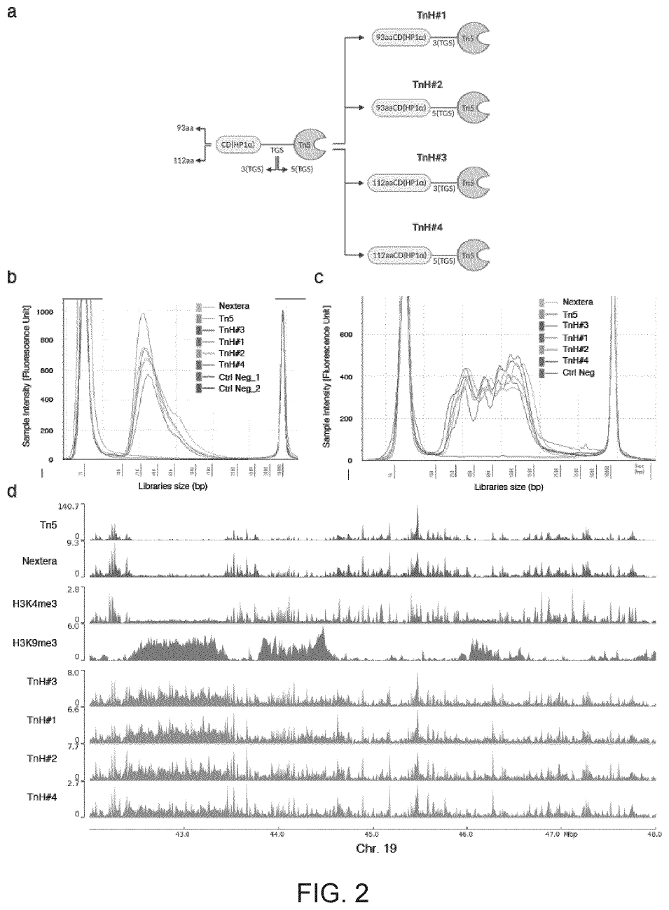

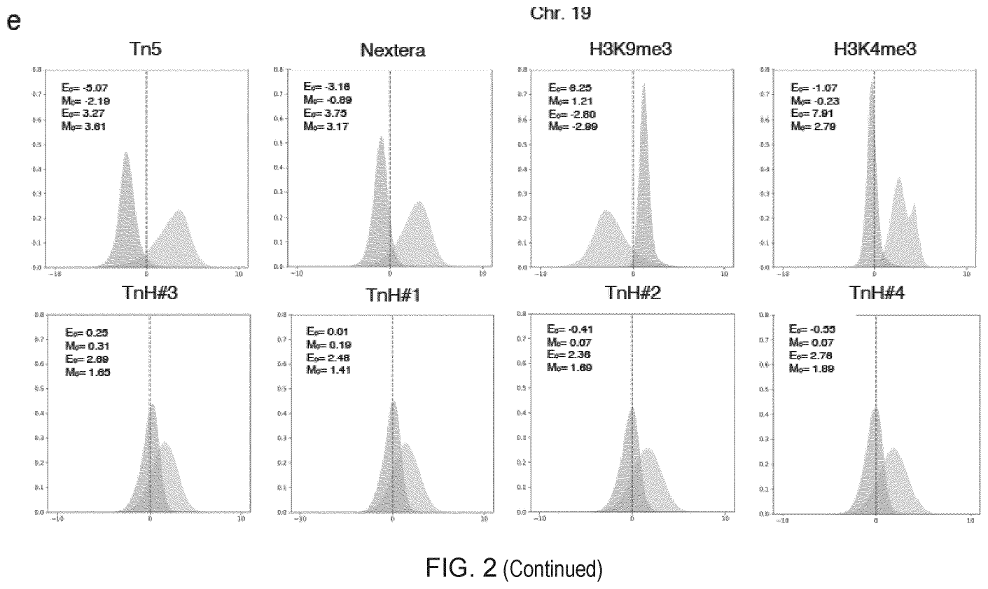

We generated a hybrid protein, whereby CD (HP1α) was cloned alongside Tn5 (FIG. 2a). We tested four constructs, generated linking two CD (HP1α)-containing regions (spanning amino acids 1-93 and 1-112) to Tn5, using either a 3 or 5 poly tyrosine-glycine-serine (TGS) linker (TnH #1-4, FIG. 2a). All four hybrid constructs were as efficient as the native Tn5 (either commercialized Nextera enzyme or in-house produced, from now on, Tn5) to fragment and insert oligos on genomic DNA (FIG. 2b).

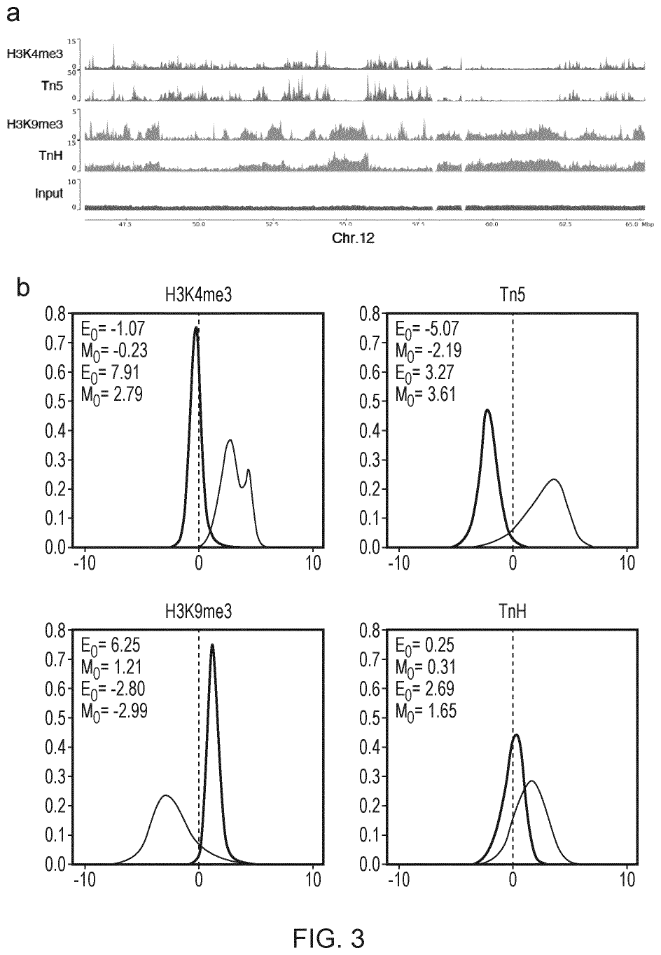

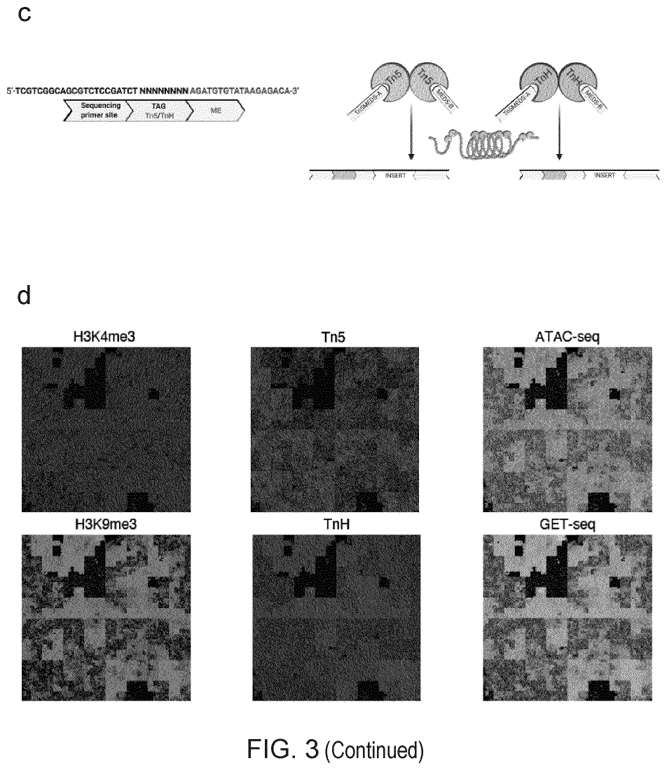

We then determined whether TnH #1-4 were able to target chromatin harbouring H3K9me3 histone modifications by tagmenting native chromatin on permeabilized nuclei (FIG. 2c). Unlike Nextera and Tn5 enzymes, hybrid Tn5 constructs indeed cut and inserted oligos in regions enriched for H3K9me3, suggesting that the CD (HP1α) redirects Tn5 towards heterochromatic regions (FIG. 3a and FIGS. 2c and d). We identified the construct TnH #3, from now on TnH, as the most efficient (FIGS. 2d and e).

Notably, TnH retained affinity toward accessible sequences as well (FIGS. 3a and b). Indeed, comparing the results of H3K9me3 and H3K4me3 ChIP-seq with TnH and Tn5 ATACseq, we observed that TnH captures both H3K4me3- and H3K9me3-enriched regions, unlike Tn5 which closely matches H3K4me3 profiles (FIGS. 1a and b).

We next reasoned that combining Tn5 and TnH in a single experiment could provide a comprehensive perspective of accessible chromatin, alongside compacted chromatin defined by H3K9me3 (FIG. 3c). We thus loaded each of the two transposases with a set of specific barcoded oligos, in order to discriminate Tn5 from TnH tagmentation products (FIG. 3c).

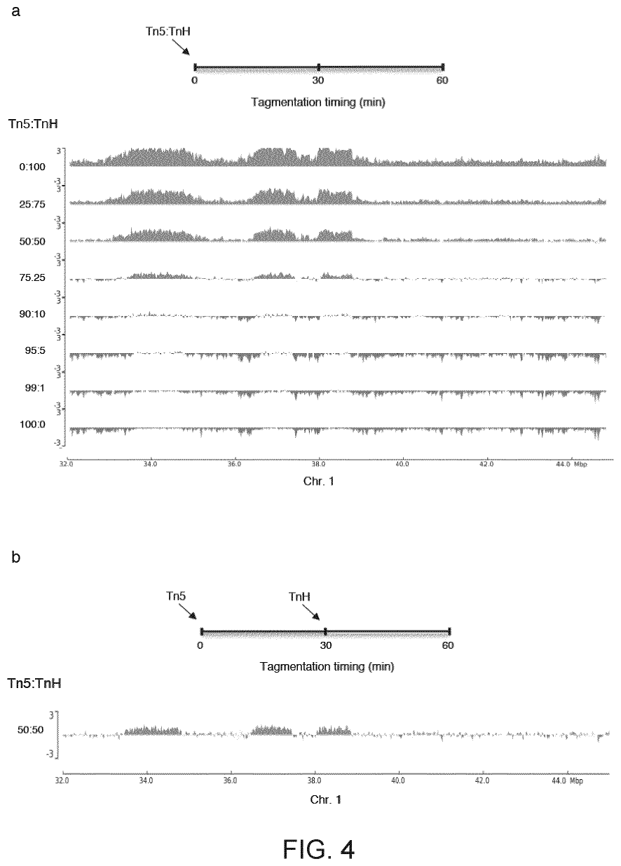

Seeking to sample either accessible or H3K9me3-labeled compacted chromatin with the highest efficiency, we tested the effect of varying the Tn5-to-TnH ratio (FIG. 4a) or adding sequentially the two enzymes (FIG. 4b) in the transposition reaction. We determined that the sequential use of the native Tn5, followed by TnH, provided the most comprehensive and accurate mapping of the two chromatin profiles. Remarkably, while the signal emerging from Tn5 overlapped closely with H3K4me3 ChIP-seq profiles, TnH mimicked more closely H3K9me3 data, and to a lesser extent H3K4me3 (FIG. 3d).

All together, these results demonstrate that a sequential combination of a Tn5 and a Tn5 which incorporates a CD derived from HP1α, TnH, is able to differentiate the signal emerging from accessible versus compacted chromatin, thus defining the whole-genome epigenetic distribution of eu- and hetero-chromatin. We nominated this method GET-seq (genome and epigenome by transposases sequencing).

### Example 3-GET-Seq can be Applied to Single-Cell Genomic Analysis (scGET-Seq) and Define Genomic Copy Number Variants at Single Cell Level

We then attempted to implement this method also to single-cell analysis. To obtain droplet-based scGET-seq, we modified the Chromium Single Cell ATAC v1 protocol (10× Genomics), replacing the provided ATAC transposition enzyme (10× Tn5; 10× Genomics) with Tn5 and TnH in appropriate enzyme proportions.

We first assessed the distribution of reads assigned to unique cell barcodes, using 10× Tn5, TnH, Tn5, or a combination of TnH and Tn5 (scGET-seq) in Caki-1 cells, and found that the 4 profiles were overlapping (FIG. 5a). We next explored the portion of the genome which was captured by each transposase. We found that TnH had the higher mean distribution of coverage per cell, with a smaller standard deviation, when compared with either Tn5 and or 10× Tn5 (FIG. 5b), suggesting indeed that even at the single-cell level TnH captures genome areas that are not targeted by conventional transposases.

Indeed, when single cell Tn5 and TnH data were each combined in pseudobulks and compared with the ChIP-seq data obtained in the same cells using H3K9me3 and H3K4me3 antibodies, we confirmed that TnH was able to target regions positive for H3K9me3 as well as H3K4me3 (FIG. 5g), as anticipated by the bulk TnH results (FIG. 3a).

We then determined whether scGET-seq was able to capture cell identity. To this end, we sequenced a mixture of the cancer cell lines HeLa and Caki-1, which originate from different tissues (cervix and kidney, respectively) and present heavily rearranged and profoundly different genome anatomies. Cells were mixed to obtain a 20:80 proportion of HeLa: Caki-1 cells.

Combining in a single UMAP embedding the scGET-seq data from the two cell lines, cells were clearly separated in two clusters sized with the expected proportions, including respectively HeLa and Caki-1 cells (FIG. 5c).

To further confirm the identity of the clusters, we used available bulk ATAC-seq data for both cell lines and generated a score for each cell line. The respective scores clearly distinguished each cell line clusters (FIG. 5c). These results were in line with what could be profiled using standard scATAC-seq (FIG. 5h).

In all, these data confirm that GET-seq could be applied to droplet-based single-cell approaches and is able to easily differentiate cells derived from different genetic backgrounds.

The definition of genomic copy number variants (CNVs) using scATAC-seq remains imprecise. It has previously been determined that only accessible chromatin regions are surveyed by this approach and the remaining genomic sequences could only be imputed from adjacent regions, thus reducing the accuracy of the measure.

As TnH targets wider portions of the genome (FIGS. 3a, 3b, 2d and 2e), we tested whether it could be exploited also to define CNVs. We first determined the global genomic landscape of HeLa and Caki-1 cells by whole genome sequencing, which showed a large fraction of the genome involved into CNVs in both cell lines (Fraction of Genome Altered, FGA: Caki-1=0.475, HeLa=0.508).

We then compared and contrasted the whole genome bulk data obtained in these cell lines with the average pseudo-bulk profile obtained with 10× Tn5 (scATAC-seq, FIG. 5e) and TnH respectively (scGET-seq, FIG. 5e). Overall, the correlation between the genomic profiles obtained with whole genome sequencing and the single-cell data was much higher for the TnH signal, when compared with 10× Tn5, at various resolutions (10 Mb, 1 Mb, and 500 kb) (FIGS. 5d and e, and FIGS. 6a and b).

A closer inspection to the segmentation profile at the single-cell level revealed that scATAC-seq is able to define CNVs but only at a coarse resolution (10 Mb), as previously determined.

Even at this resolution, scGET-seq, using TnH, showed a much higher consistency, for both cell lines, than 10× Tn5 (FIG. 6b). Increasing the resolution, up to 500 kb, scGET-seq remained reliable while the ability of scATAC-seq to identify CNVs degraded considerably, as large swaths of the genome were excluded from the analysis (FIGS. 5e and 6a). In fact, the signal emerging from scATAC-seq correlated closely with the location of regulatory elements throughout the genome, which was not the case for scGET-seq (FIG. 5f).

We tested the ability of scGET and 10× to call actual CNA events (amplification, deletion and normal status) using a machine learning approach. To this end we called CNA from bulk WGS sequencing of Caki and Hela cells. We then split scGET-seq and scATAC-seq genomic bins into training and test sets (proportion 70:30) and trained a logistic regression classifier (LR) and a Support Vector Machine with linear kernel (SVM). We calculated their accuracy and F1-score on the test set. The results are shown in FIG. 5i, and indicate that scGET-seq performs better than scATAC-seq regardless of the classifier and the resolution. We found that the performance depends on the number of cells included in the analysis, as in Hela cells, which were approximately ⅕ with respect of the Caki-1 cells, as the classifier is given less data in the training step.

All together, these results suggest that scGET-seq can be successfully used to concomitantly obtain detailed information on the single-cell epigenetic landscape as well on the underlying genomic structure.

### Example 4-scGET-Seq Defines the Genomic and the Epigenetic Landscape of Cancer Clones Resistant to Drug Treatment

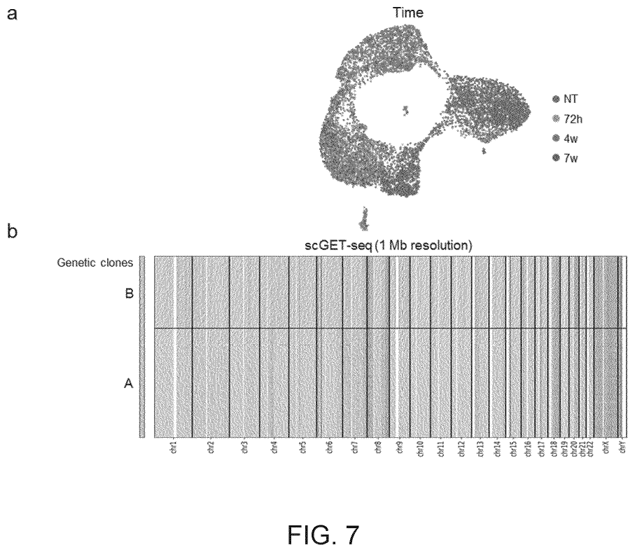

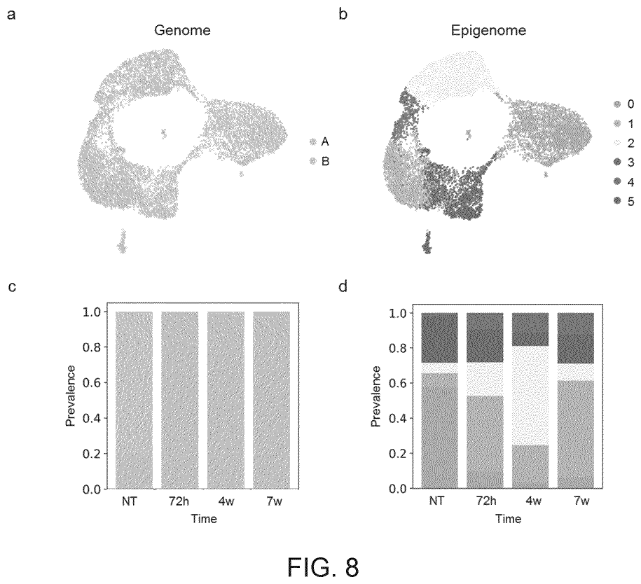

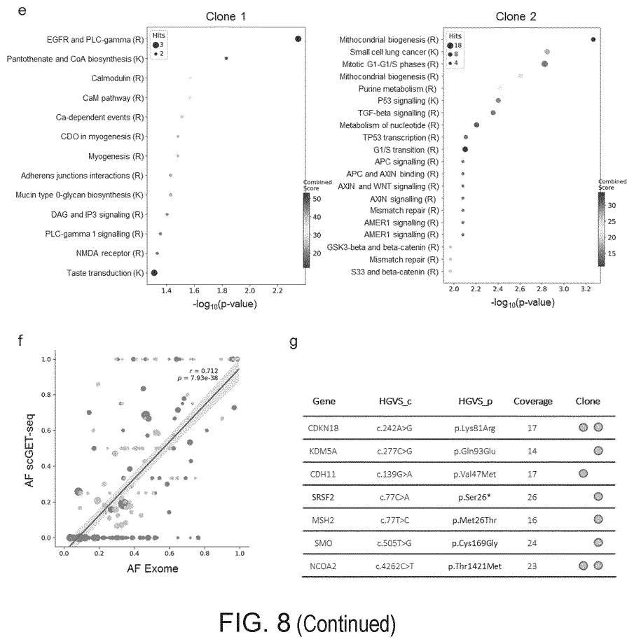

To exploit the ability of scGET-seq to capture the genomic and epigenetic landscape of single cells, we used a model system based on patient derived xenograft (PDX) models of colon carcinoma. In this setting, we have shown that resistance to therapy may arise from the selection of clones endowed with specific genetic lesions, alongside with features of plasticity that are not driven by genomic modifications but most likely by chromatin reshaping. We hence followed cancer evolution in one PDX model throughout several weeks of treatment with the clinically approved EGFR antibody cetuximab (FIG. 7a). Analysis of genomic segmentation by scGET-seq revealed 2 major clones in the absence of treatment (FIGS. 8a and c, and FIG. 7b). Conversely, cells were separated into 6 different clones when assessing the pre-treatment epigenetic landscape (FIGS. 8b and d). When the impact of treatment was assayed, clone A was predominant, while clone B was present at very low frequency (FIG. 8c). In contrast, the epigenetic landscape of cetuximab-treated PDX samples was more heterogenous, with epigenetic subclones embedded within genetic clones (FIG. 8d).

We next sought to identify processes that might provide biological insights into epigenetic mechanisms of resistance to EGFR blockade. To this end, we performed functional enrichment analysis using the genes associated to the DNase I hypersensitive sites (DHS) differentially affected in the various clones. In the epigenetic clones most associated with resistance, there was a significant enrichment on pathways associated with resistance to EGFR inhibitors, including the phospholipase C pathway, which resides downstream of the EGFR receptor and whose deregulated expression has been proposed as a mechanism exploited by cancer cells to withstand EGFR inhibition, TGFb signaling and the WNT pathway (FIG. 8e). These results are in line with our previous observations, that cancer cells exposed to targeted therapies do show resistance patterns which are not only linked to specific genetic clones but are related to genomic plasticity phenotypes most likely driven by chromatin remodeling phenomena.

As scGET-seq includes sequences for portion of the genome that are eluded by conventional ATAC-seq, we next sought to determine whether we could also define single nucleotide variations (SNV) within single cells. While not all exome SNVs were captured by scGET-seq, nonetheless there was a highly significant correlation between the mutations identified by bulk exome sequencing conducted on the primary tumor, and the scGET-seq results (FIG. 8f). scGET-seq was also able to identify mutations in cancer genes that were not present in the initial bulk exome sequencing in the starting sample. Of note, there were mutations in established cancer genes (tier 1, COSMIC Cancer Gene Census, version 92) (Sondka, Z. et al. (2018) Nat. Rev. Cancer, 18:696-705) such as CDKN1B, KDM5A, CDH11, SRSF2, 321 MSH2, SMO and NCOA2 (FIG. 8g); the enrichment for COSMIC mutations was significant for variants profiled at high depth (higher than 15; Odds Ratio=1.55, p=3.57.10-3, Fisher's exact test). At this stage, it remains to be ascertained whether the mutations that were found by single-cell analysis but not by bulk sequencing were developed de novo by the PDX or were already present in the original population at frequencies too low to be detected by the limited coverage of exome sequencing. Importantly, by virtue of the single-cell analysis, it was possible to ascribe the mutations to specific clones.

In all, these results suggest that scGET-seq could be used to comprehensively assess the tumor genome (including both CNVs and SNVs) and the epigenome, illuminating paths of cancer evolution, clonality, and drug resistance.

### Example 5—scGET-Seq Captures Chromatin Status at the Single-Cell Level

We next aimed to determine whether scGET-seq might capture the dynamic between accessible and compacted chromatin at the single-cell level. We have recently demonstrated that the ablation of the histone demethylase Kdm5c hampers H3K9me3 deposition impairing heterochromatin assembly and maintenance in NIH-3T3 cells.

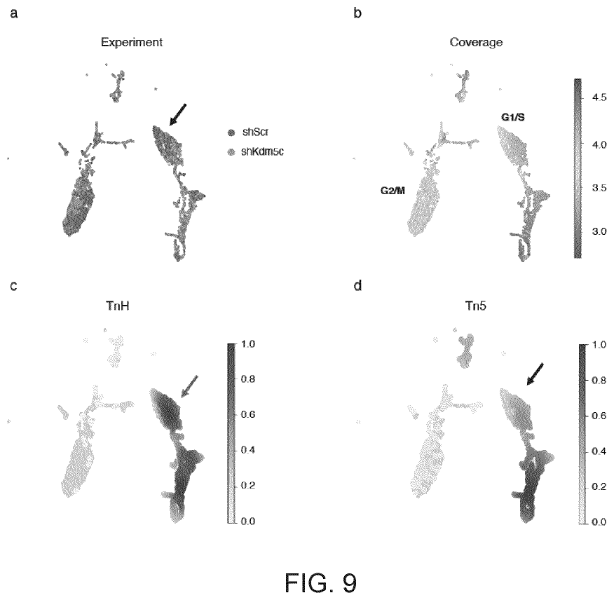

We performed scGET-seq in cells before and after Kdm5c knock-down. We identified two neatly distinguished cell groups, including shScr and shKdm5c cells, respectively (FIG. 9a). Seeking to find an explanation for this pattern, we discovered that this distinction was driven by the total number of reads per cell (FIG. 9b). We surmised that this pattern might be driven by the cell cycle status, namely, high coverage associated with cells in the S and G2/M cell, during or after DNA replication, while low coverage linked to cells in the G1 cycle phase, before the replication of DNA.

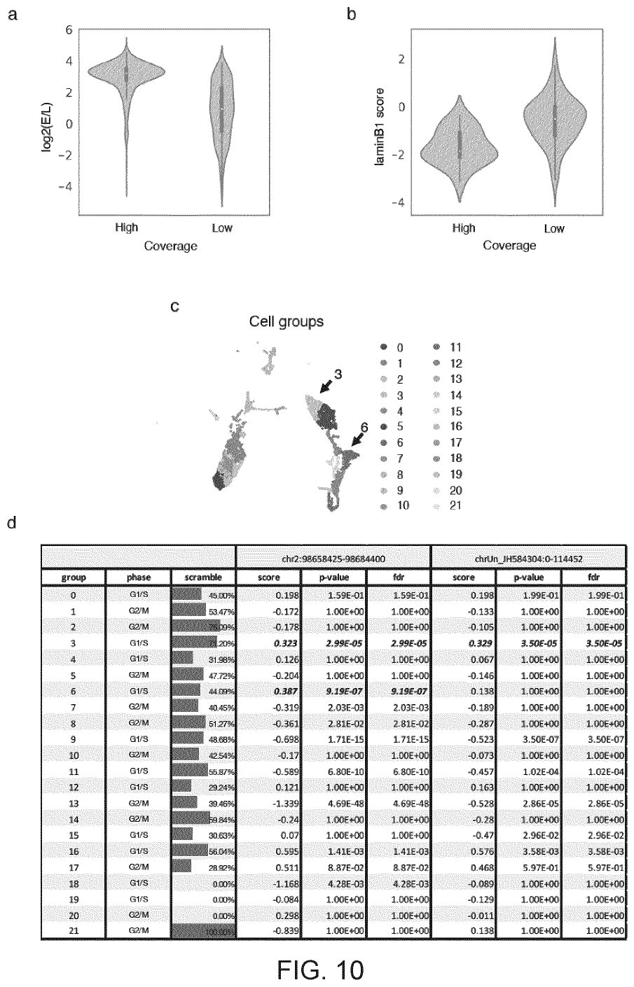

To test our hypothesis, we applied a strategy derived from Buenrostro, J. D. et al. (Buenrostro, J. D. et al. (2015) Nature, 523:486-490), where we analyzed the distribution of Repliseq (Peric-Hupkes, D. et al. (2010) Mol. Cell, 38:603-613; Hiratani, I. et al. (2008) PLOS Biol., 6:2220-2236; Marchal, C. et al. (2018) Nat. Protoc., 13:819-839) signal over differentially enriched DNase I hypersensitive sites (DHS) regions between high- and low-coverage cells. We found that high coverage cells are characterized by higher and less variable fraction of early-replicating regions (FIG. 10a), in contrast to the highly variable values characterizing the low-coverage cells. This pattern suggests that cells with high coverage are indeed in mitosis. This pattern is mirrored and confirmed by the scores calculated on laminB1 associated domain data (FIG. 10b; Peric-Hupkes, D. et al. (2010) Mol. Cell, 38:603-613).

To decode the relationship between accessible and compacted chromatin as captured by scGET-seq, we focused our analysis on major repeats, regions of the genome which undergo compaction during the cell cycle, through the acquisition of H3K9me3 residues. As Kdm5c acts, and heterochromatin assembly occurs, during the middle/late S phase we focused on the G1/S cell cycle phase. The signal emerging from Tn5 was weaker on G1/S cells where Kdm5c was not knocked down (FIGS. 9a and d, black arrow, compared with TnH, FIG. 9c, red arrow), likely because these cells present a normal assembly of H3K9me3 and heterochromatin, and therefore Tn5 would be unable to tag the resulting compacted DNA. Conversely, the signal from TnH showed a more even distribution on G1/S cells, irrespectively of Kdm5c status, as TnH targets both accessible and compacted chromatin (FIG. 9c).

We tested whether our observation was statistically significant fitting a linear model that considers the enrichment over TnH and Tn5 as interaction term when looking for groupwise specific markers. We found that the TnH enrichment was significantly higher than Tn5 in groups 3 and 6 (FIGS. 10c and d), where indeed shKdm5c cells are present in higher percentage, suggesting that TnH is able to selectively capture regions of the genome, such as chromatin decorated with H3K9me3, which Tn5 is unable to reach.

All together, these data suggest that GET-seq pinpoints quantitative differences between the two enzymes arising from the local chromatin status.

### Example 6—scGET-Seq Identifies the Trajectories of Fibroblasts Reprogramming Towards iPSC and of iPSC Differentiation Towards Neural Progenitor Cells

H3K9 and chromatin compaction profoundly modulate development and reprogramming. We thus explored the potential role of scGET-seq in illuminating these processes. To this end, we explored the single-cell profiles of cultured fibroblasts (FIB) obtained from two healthy subjects, undergoing reprogramming into induced pluripotent stem cells (iPSC), and of iPSC undergoing differentiation into neural progenitor cells (NPC).

scGET-seq distinguished FIB, iPSC and NPC into three distinct populations (FIG. 11a). Notably, the 3 populations were connected in a continuum, suggesting that scGET-seq is able to capture also cells in transition between states. Specifically, the groups 4, 5, 6, 8, 10 and 11 represented cells in transition among the three major states (FIG. 11b).

We then combined the scGET-seq data from both individuals. While both FIB and iPSC derived from the two donors were mixed (FIG. 11c), NPCs from each donor were neatly separated, reflecting the in vitro assessment of the cells, which had revealed that NPC derived from donor A differentiated faster towards the neural lineage, compared to NPC derived from donor B.

We next attempted to define the differentiation potential (DP) of each cell using Palantir (Setty, M. et al. (2019) Nat. Biotechnol., 37:451-460). DP represents the probability of a single cell to fall into different branching trajectories, irrespectively of its developmental phase. We found that a large subset of FIB was endowed with the highest DP. DP then decreased progressively in iPSC and even more in NPCs (FIG. 11d). Intriguingly, there were subsets of FIB and iPSC which demonstrated an exceedingly low DP. In particular, iPSC diverged into two populations, one linked to NPCs and progressing towards that fate, and a second one reaching a status apparently lacking differentiation potential.

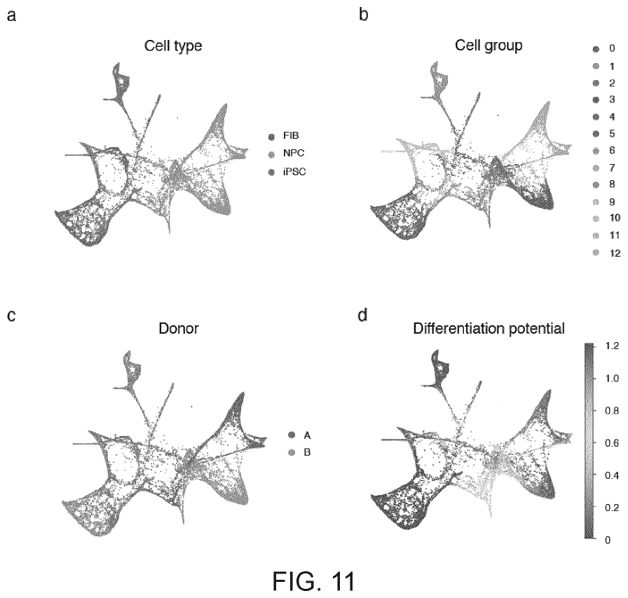

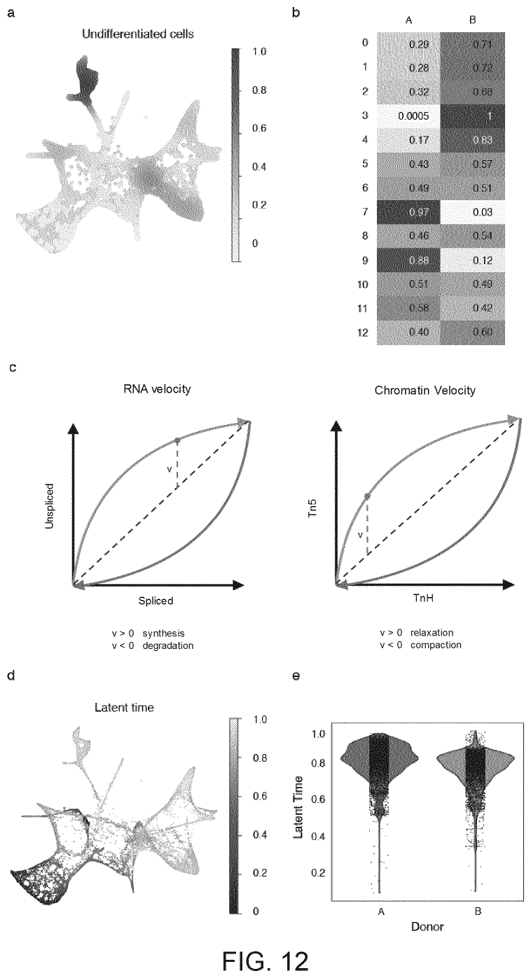

We verified properties of the iPSC cells unable to differentiate by integrating a signature of undifferentiated iPSC35 into our data (FIG. 12a). We confirmed that the iPSC with low DP are, in fact, a group of cells that do not progress during differentiation. To further strengthen this notion, we also found that iPSC belonging to donor B were enriched in this cluster (72% donor B versus 28% donor A, FIG. 12b).

### Example 7—Chromatin Velocity to Define Epigenetic Vectors

Prompted by the quantitative properties of scGET-seq highlighted in the shKdm5c experiment, we sought to investigate developmental dynamics in terms of differential compaction of chromatin. RNA velocity is a tool recently introduced which uses scRNA-seq data to capture not only the overall developmental direction of each cell, but also its kinetics, that is, the differential displacement by which the various cells travel through states. We hence explored whether it is feasible to obtain single cell trajectories using scGET-seq data. To this end, instead of using the ratio between unspliced and spliced mRNA (as in RNA-velocity), we exploited the ratio between Tn5 and TnH signals, at any given location, under the assumption that an increase in this value points to a dynamic process leading to a more relaxed chromatin, while the opposite is indicative of chromatin compaction (FIG. 12c).

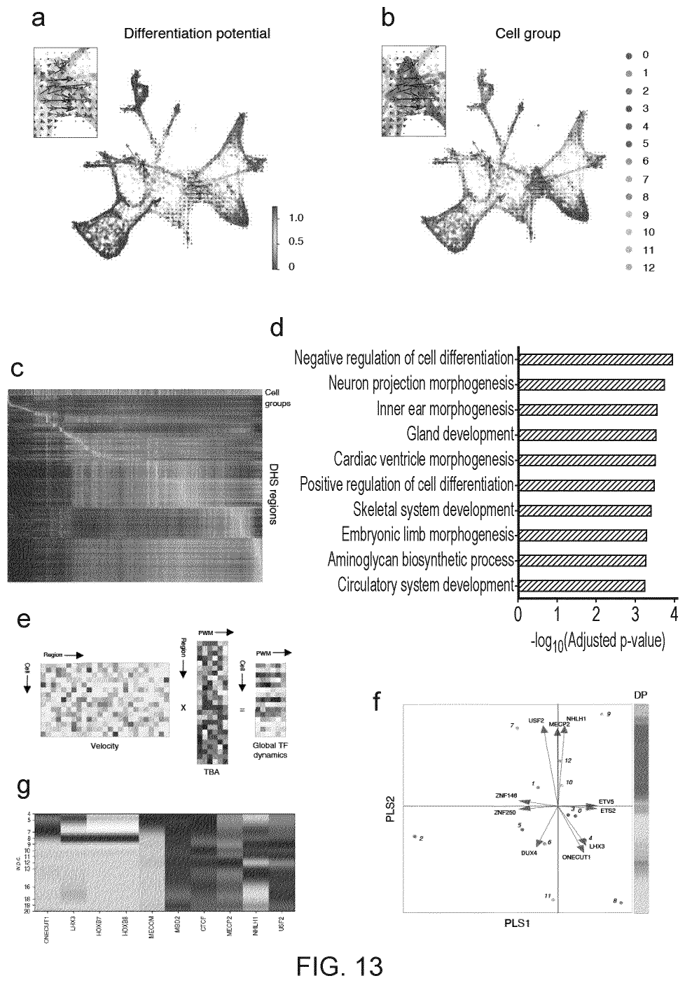

We found that this approach, which we named Chromatin Velocity, is indeed able to capture not only the overall direction but also the velocity of chromatin remodelling (FIG. 13a). According to this analysis, and in line with the DP analysis, most FIB are static, that is, they do not show any appreciable flux. iPSC themselves could be divided in 2 main populations. An iPSC subset rapidly moves towards NPCs (cell groups 6, 11 and 8), while a second group enters in a more static condition, which did not differentiate towards NPCs (group 5) and lacked clear directionality. The most critical juncture, where cells enter into a “chute”, encompasses groups 8 and 4 (FIGS. 13a and b, insets), which includes only NPCs moving towards their final destination, where they land into a static, low DP condition (groups 3, 7, 9 and 12). Analysis of chromatin velocity with a full dynamical model allows the estimation of Latent Time (LT, FIG. 12d; Bergen, V. et al. (2020) Nat. Biotechnol. doi: 10.1101/820936). Interestingly, NPC cells from donor A show higher values of LT, consistent with the observation they differentiate faster (FIG. 12e).

In all, these results reveal that the transition from FIB to iPSC and finally NPC is not characterized by a constant developmental speed, but includes critical junctions, featuring variable speeds, with some brisk acceleration passages during the differentiation from iPSC to NPC.

Curious to find the pathways engaged in the differentiation process, in particular during these “chutes”, we analyzed the results of the dynamical model and identified the 1,655 DHS regions with highest likelihood of being subjected to remodelling during the transition from FIB to iPSC and NPC (FIG. 13c). The functional analysis on the genes associated to these regions revealed a strong enrichment for categories related to cell differentiation and neural morphogenesis, suggesting that our approach is indeed able to grasp biological processes relevant to the model (FIG. 13d).

As transcription factors (TF) are the key drivers of differentiation, we designed a global TF dynamic score (FIG. 13e and methods), a cell-by-TF value that is informative of the role of specific TF in specific cell trajectories. Intriguingly, a Partial Least Square regression (PLS) analysis of this matrix revealed that PLS2 was strongly associated with DP (FIG. 13f). Indeed, the most differentiated NPCs (belonging to donor A, cell groups 7, 9 and 12) were strongly enriched for TFs which are key for neural differentiation, namely NHLH138 and MECP2, whose mutations lead to mental retardation. Notably, it has been recently shown how MeCP2 enhances the separation of heterochromatin and euchromatin through its condensate partitioning properties. Conversely, other groups were on the opposite, endowed with an intense differentiation potential, including cell groups 4 and 8, which feature the bottleneck of swift chromatin velocity described above. Two TFs were pivotal in these cells, ONECUT1 and LHX3. It has been recently shown that ONECUT1, alongside its homologs, elicits a widespread remodelling of chromatin accessibility, thus inducing a neuron-like morphology and the expression of neural genes. Importantly, ONECUT1 and LHX3, alongside ISLET1, tightly cooperate to dictate the transition from nascent towards maturing ESC-derived neurons through the engagement of stage-specific enhancers.

As PLS2 seems to be associated to the development stage of neural cells, we assessed whether a similar pattern is recapitulated in vivo. To this end, we analyzed expression data of developing human brain obtained from Cardoso-Moreira, M. et al. (Cardoso-Moreira, M. et al. (2019) Nature, 571:505-509), focusing on the early time points (4-20 weeks post conception). With the exception of DUX4, which was not profiled in that dataset, we found that TF with the most negative loading on PLS2 have a single peak of expression in the early stages of brain development (FIG. 13g) and are abruptly downregulated afterwards. Conversely, TF presenting the most positive loading show a sustained expression, which peaks at later time points.

All together, we posit that Chromatin Velocity captures epigenetic transitions underlying crucial biological processes and illuminates the hidden transcription factor networks and wiring driving these dynamic fluxes.

The following Examples represent additional, complementary experiments:

### Example 8—GET-Seq Identifies Clonality in Patient-Derived Organoids

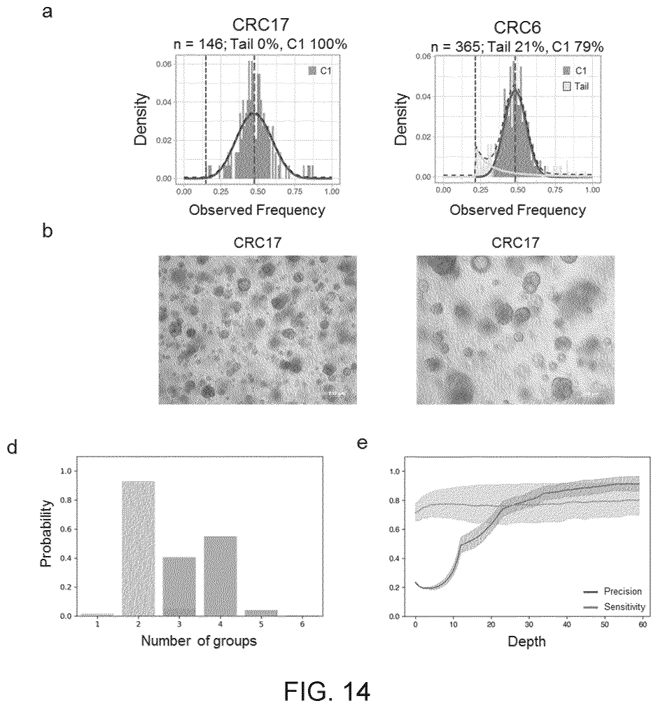

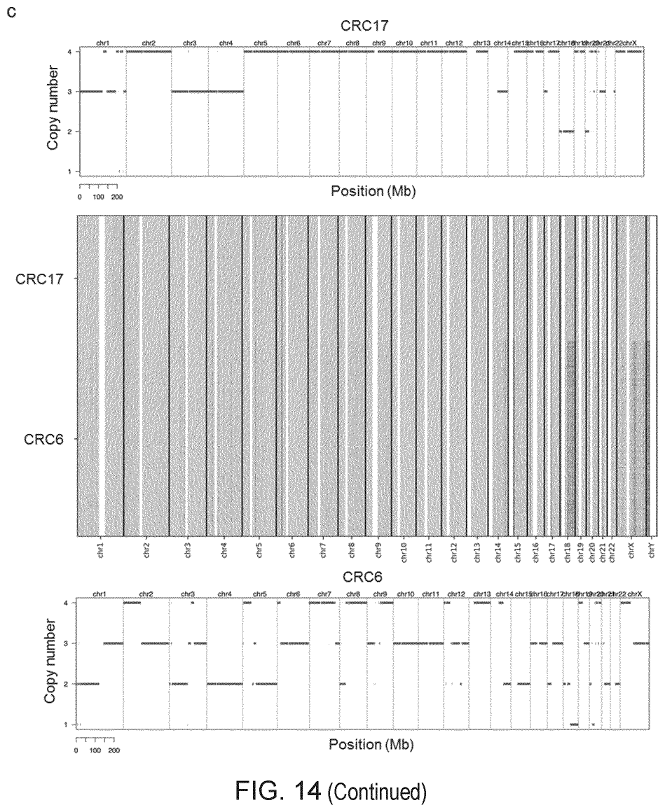

To ascertain the ability of GET-seq to define clonality, we decided to rely on a more physiological experimental setting than cell lines, patient derived organoids (PDOs). We thus used a tumour matched-normal design to generate whole-exome data derived from two hepatic metastases of primary colorectal tumours (CRC 6 and 17). The analysis of somatic single nucleotide variants and allele-specific copy numbers showed high-level of aneuploidy for both samples, with a triploid (CRC6) and a tetraploid (CRC17) tumour genome. From the analysis of allele frequency spectra and cancer cell fractions we found no evidence of ongoing subclonal expansions, concluding that CRC6 and CRC17 are monoclonal, a common characteristic of late stage colorectal cancers (Cross, W. et al. (2018) Nat. Ecol. &amp; Evol., 2:1661-1672; Cross, W. et al. (2020) bioRxiv, doi: 10.1101/2020.03.26.007138) (FIG. 14a). From these samples we generated PDOs (FIG. 14b), which we then profiled with scGET-seq. We analyzed data from both samples simulating a 1:1 mixture under the hypothesis that a scGET-seq analysis should only reveal two clones, one for each organoid. Indeed, the CNV analysis confirmed the existence of two main cellular populations, with defining genomic features, closely mimicking the two CRC6 and 17 cancer populations (FIG. 14c). To provide quantitative support to this observation, we also calculated the posterior marginal probability distribution of the number of observable clones. This analysis confirmed that scGET-seq could correctly identify 2 clusters, corresponding to CRC6 and CRC17. Notably, only a minority of the cells assessed were misclassified. A similar analysis on Tn5-derived reads showed a tendency for overclustering and of cell misclassification (FIG. 14d). We finally explored the accuracy of variant calling (i.e. presence/absence of a variant) by comparing genotyped clones with known variants profiled in the bulk samples. We evaluated the dependency of precision and sensitivity at different depth thresholds and we found these metric are in line with previous observations (Gézsi, A. et al. (2015) BMC Genomics, 16:875) although values are slightly smaller and sample-dependent (FIG. 14e).

All together, these results suggest that scGET-seq can be successfully used to concomitantly obtain detailed information on the single-cell epigenetic landscape as well on the underlying genomic structure.

### Example 9—scGET-Seq Defines Cell Identity and Identifies Developmental Trajectories of Fibroblasts Reprogramming Towards iPSC and of iPSC Differentiation Towards Neural Progenitor Cells (Related to Example 6)

The modulation of H3K9 methylation and chromatin compaction are pivotal mechanisms underlying organismal development and cellular reprogramming. We thus explored the potential role of scGET-seq in illuminating these processes. To this end, we explored the single-cell profiles of cultured fibroblasts (FIB) obtained from two unrelated healthy subjects, undergoing reprogramming into induced pluripotent stem cells (iPSC), and of iPSC undergoing differentiation into neural progenitor cells (NPC). In parallel, we performed scRNA-seq analysis on cells from the same samples.

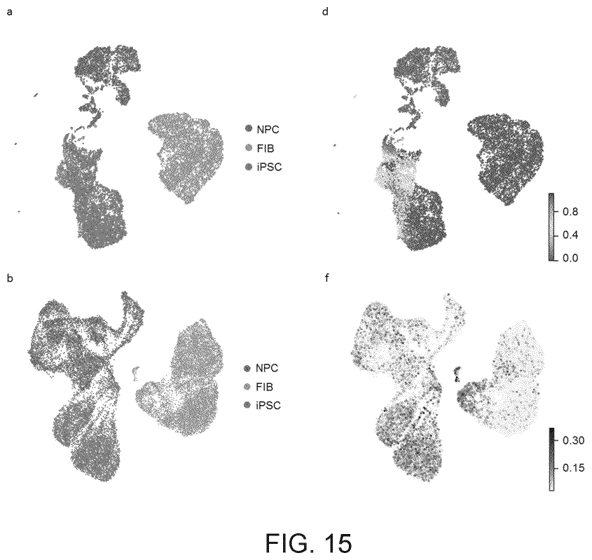

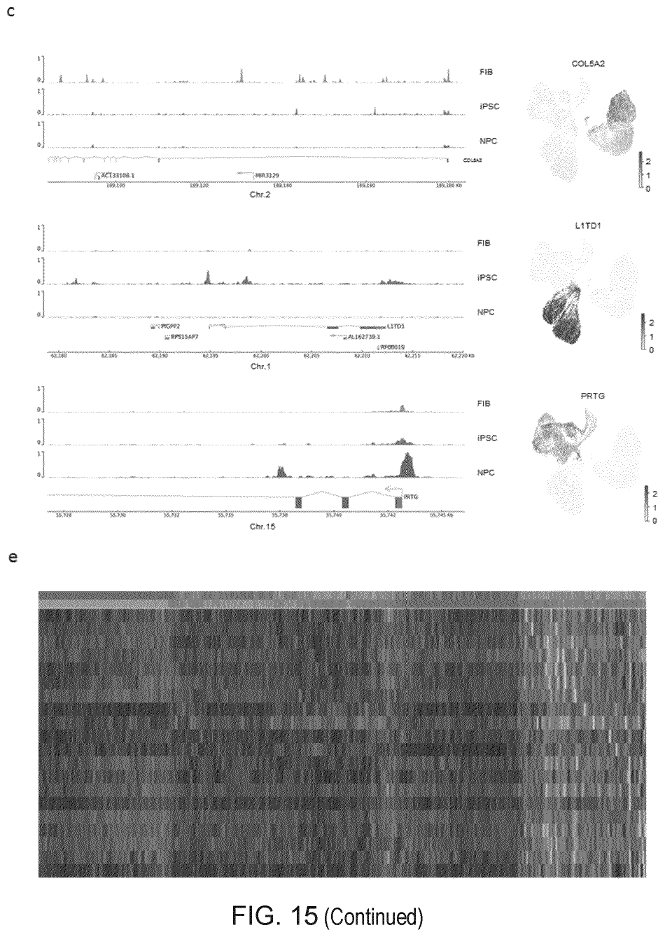

Low dimensional representation of single cell data from scGET-seq and scRNA-seq separated FIB, iPSC and NPC into three distinct populations (FIGS. 15a and b). Notably, UMAP representations of both scGET-seq and scRNA-seq data showed that iPSC and NPC were in close proximity, while FIB were isolated from the other two populations, with the exception of a small subset of FIB and to a lesser extent NPCs clustering alongside iPSC exclusively in the scGET-seq data (black arrow in FIG. 15a).

We next explored the genomic regions more closely defining each population. Notably, the GET-seq sequences most significantly enriched in each cell type were in proximity of genes which are crucial for the biology of each population, such as collagen for FIB, L1TD1 for iPSC37 and PRTG for NPC38 (FIG. 15c), with concomitant expression in the corresponding populations.

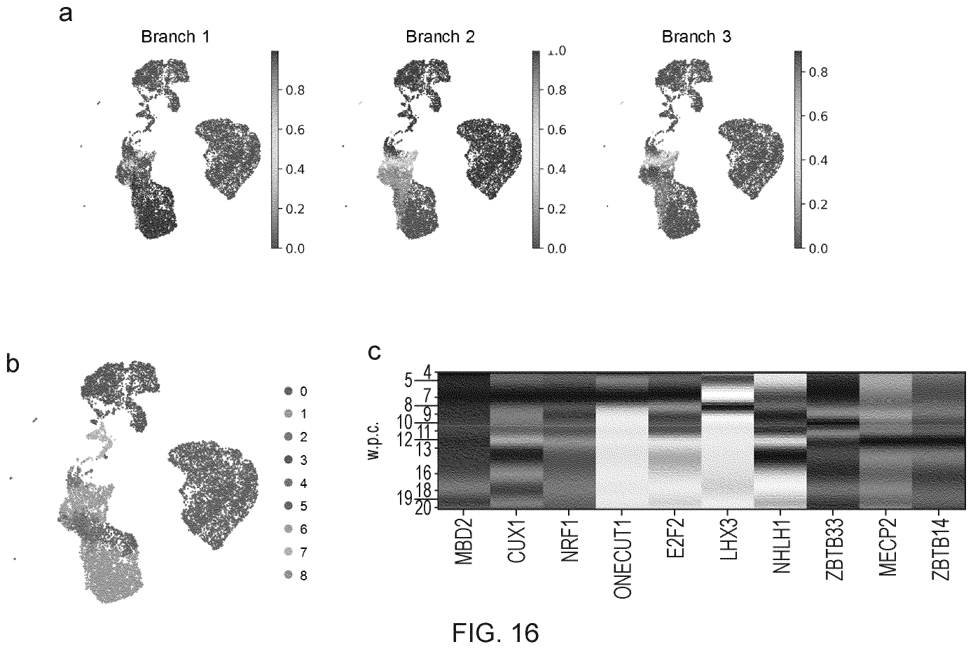

We next sought to determine whether the epigenetic landscapes depicted by scGET-seq could be exploited to capture cell fate probabilities. We surmised that the transition from FIB to iPSC and ultimately to NPC provides an ideal tool to test this hypothesis. Indeed, it has been recently proposed that cell fate choices are driven by a continuum of epigenetic choices, more than a series of discrete bifurcation alongside developmental paths (Setty, M. et al. (2019) Nat. Biotechnol. 37, 451-460). To this end, a tool has been recently devised, Palantir, which is able to capture these dynamics from scRNA-seq data. When we applied Palantir to the GET-seq data set, we found three main fate branches (FIG. 16a) defining a group of cells endowed with an intense differentiation potential (FIG. 15d), which included iPSC and the subset of FIB and NPC clustering alongside iPSC (FIG. 15a).

Intrigued by these results, we then explored the regions defining these cellular populations endowed with the highest differentiation potential (FIG. 15e). Surprisingly, we found that these regions resided for the most part in pericentromeric regions, in line with recent reports supporting a crucial role for these genomic areas as drivers of pluripotency Wang, C. et al. (2018) Nat. Cell Biol. 20, 620-631; Nicetto, D. & Zaret, K. S. (2019) Curr. Opin. Genet. Dev. 55, 1-10; Burton, A. et al. (2020) Nat. Cell Biol. 22, 767-778; Novo, C. L. et al. (2016) Genes Dev. 30, 1101-1115). We hence used the genes associated to these regions to generate a differentiation signature, which we then applied to scRNA-seq data. Remarkably, this signature highlighted in the scRNA-seq data a subset of NPC as well as FIB, which were the closest FIB to iPSC, sharing similar features (red arrows in FIG. 15f).

In all, these results suggest that GET-seq is able to capture the epigenetic diversity arising during developmental processes and to identify key factors engaged in the process. Additionally, this approach may uncover epigenetic events arising before the appearance of the concomitant transcriptomic events.

### Example 10—Chromatin Velocity to Define Epigenetic Vectors (Related to Example 7)

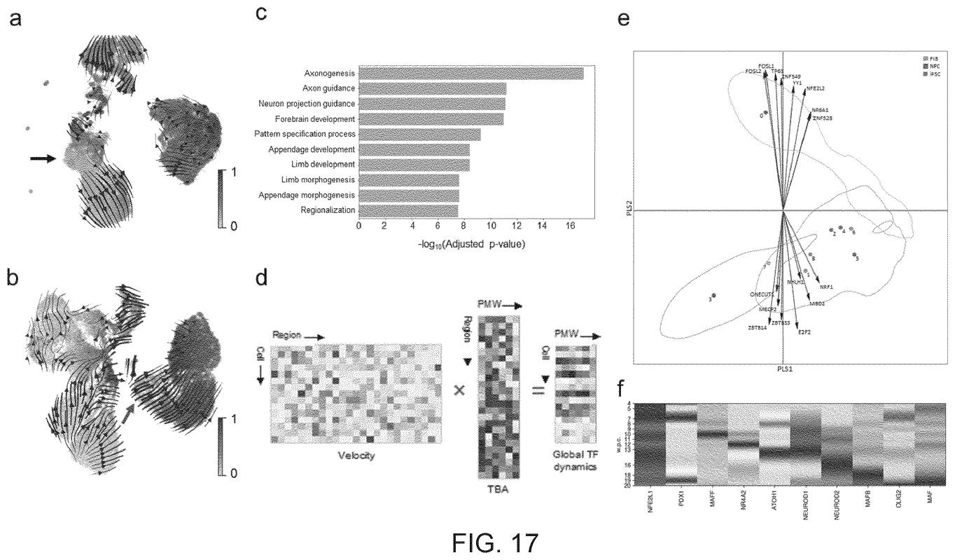

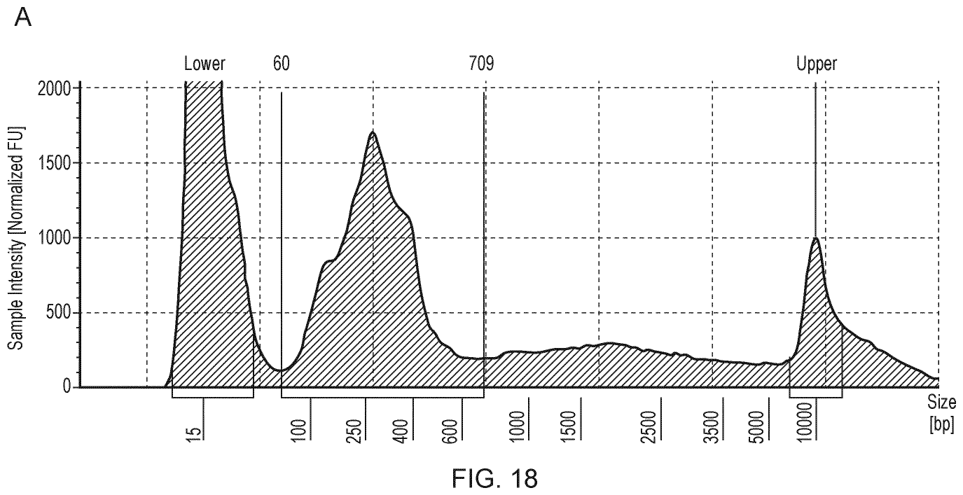

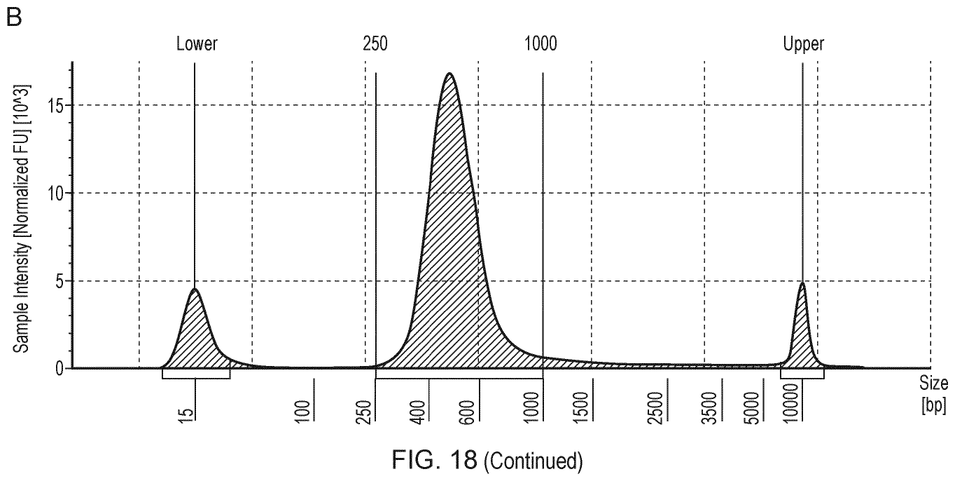

Prompted by the quantitative properties of scGET-seq highlighted in the shKdm5c experiment, we sought to investigate developmental dynamics in terms of differential unfolding of chromatin. RNA velocity is a tool recently introduced which uses scRNA-seq data to capture not only the overall developmental direction of each cell, but also its kinetics, that is, the differential displacement by which the various cells travel through states. We hence explored whether it is feasible to obtain single cell trajectories using scGET-seq data. To this end, instead of using the ratio between unspliced and spliced mRNA, as in RNA-velocity, we exploited the ratio between Tn5 and TnH signals, at any given location, under the assumption that an increase in this value points to a dynamic process leading to a more relaxed chromatin, while the opposite is indicative of chromatin compaction (FIG. 12c). We found that this approach, which we named Chromatin Velocity, is indeed able to capture not only the overall direction but also the velocity of chromatin remodeling (FIG. 17a), with a pattern similar to RNA-velocity (FIG. 17b). Of note, the overall pattern of chromatin velocity recapitulates Palantir results in highlighting a group of cells including iPSC, NPC and FIB from which most differentiation processes appeared to arise (FIGS. 17a and 15d). Also, RNA-velocity revealed that the subset of FIB enriched for the differentiation signature represented the origin from which the FIB population arose (FIG. 17b).

Curious to find the pathways engaged in the differentiation process, we analyzed the results of the dynamical model and identified the 1,703 DHS regions with highest likelihood of being subjected to remodeling. The functional analysis on the genes associated to these regions revealed a strong enrichment for categories related to neural morphogenesis, including axonogenesis and various pathways linked to neural development and morphogenesis, suggesting that our approach is indeed able to grasp biological processes relevant to the model (FIG. 17c).

As transcription factors (TF) are the key drivers of differentiation, we designed a global TF dynamic score (FIG. 17d and methods), a cell-by-TF value that is informative of the role of specific TF in specific cell trajectories. We applied a Projection to Latent Structures regression analysis (PLS) (Wold, S., et al. (2001) Chemom. Intell. Lab. Syst. 58, 109-130) fitting the cell TF scores to cell clusters (FIG. 16b) which clearly separated FIB on one site, and NPC and iPSC on the other. Several TFs already implicated in FIB development and maintenance were included, such as FOSL246, TP6347, and NFE2L248. Conversely, NPCs and iPSC were strongly enriched for TFs which are key for neural differentiation, namely NHLH149 and MECP2, whose mutations lead to mental retardation. MECP2, MBD2 e ZBTB33 (KAISO) exert redundant activities in neuronal development. Notably, it has been recently shown how MECP2 enhances the separation of heterochromatin and euchromatin through its condensate partitioning properties. Two TFs were pivotal in these cells, ONECUT1 and LHX3. It has been recently shown that ONECUT1, alongside its homologs, elicits a widespread remodeling of chromatin accessibility, thus inducing a neuron-like morphology and the expression of neural genes. Importantly, ONECUT1 and LHX3, alongside ISLET1, tightly cooperate to dictate the transition from nascent towards maturing ESC-derived neurons through the engagement of stage-specific enhancers.

As PLS1 seems to be associated to the development stage of neural cells, we assessed whether a similar pattern is recapitulated in vivo. To this end, we analyzed expression data of developing human brain obtained from Cardoso-Moreira, et al. (Cardoso-Moreira, et al. (2019) Nature 571, 505-509), focusing on the early time points (4-20 weeks post conception). With the exception of DUX4, which was not profiled in that dataset, we found that TF with the most negative loading on PLS1 have a single peak of expression in the early stages of brain development (FIG. 17f) and are abruptly downregulated afterwards. Similarly, TF with the most negative loading on PLS2 include many entries that are also active in the very early stages of brain development (FIG. 16c), such as MBD2, ONECUT1 and LHX3.

All together, we posit that Chromatin Velocity captures epigenetic transitions underlying crucial biological processes and illuminates the hidden transcription factor networks and wiring driving these dynamic fluxes.

In summary, we propose a new method, scGET-seq, that captures genomic and chromatin landscapes and trajectories, as well as key players, which could provide important insights in fields as diverse as development, regenerative medicine and the study of human diseases, including cancer.

### Example 11—TnH Transposase for Multiomic Application

Hybrid transposase TnH, in combination with transposase Tn5, was used to develop a novel multiomic approach to capture RNA, and accessible and compacted chromatin (building on the established GET-seq approach) on droplet based microfluidic platform (Chromium Single Cell Multiome ATAC+Gene Expression kit, 10× Genomics Chromium).

For this approach, the TnHMEDS-A and Tn5MEDS-A oligonucleotides were modified to include a 5′-phospate group (named multiMEDS-A) in order to allow binding of tagmentation protocol to the capturing hydrogel beads (part of the Chromium Single Cell Multiome ATAC+Gene Expression kit, 10× Genomics), obtaining the new Tn5-multi and TnH-multi complexes. The hydrogel beads contain also the polyA capture probe.

This approach, named GET²-seq, was tested on Caki-1 cell line using the Chromium Single Cell Multiome ATAC+Gene Expression kit (10× Genomics), producing good quality libraries for sequencing.

10′000 cells were used as input for the experiment. As for the standard GET-seq protocol, tagmentation reaction was started by adding Tn5-multi, while TnH-multi complex was added after 30′ and tagmentation reaction continued for a total of 1 h incubation.

All publications mentioned in the above specification are herein incorporated by reference. Various modifications and variations of the described products, methods and uses of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for making a DNA sequence library or libraries comprising the steps:
a) providing a sample comprising genomic DNA;
b) adding at least one engineered transposome complex;
c) optionally amplifying tagged DNA;
d) optionally isolating the amplified DNA; and
e) optionally sequencing tagged DNA, the amplified DNA or the isolated DNA.

2. A method for DNA sequencing comprising the steps:
a) providing a sample comprising genomic DNA;
b) adding at least one engineered transposome complex;
c) optionally amplifying tagged DNA;
d) optionally isolating the amplified DNA; and
e) sequencing tagged DNA, the amplified DNA or the isolated DNA.

3. A method for making a DNA sequence library or libraries and an RNA sequence library or libraries comprising the steps:
a) providing a sample comprising genomic DNA and RNA;
b) (i) adding at least one engineered transposome complex; and
(ii) tagging the RNA;
c) optionally amplifying tagged DNA and/or tagged RNA;
d) optionally isolating the amplified DNA and/or the amplified cDNA; and
e) optionally sequencing tagged DNA, the amplified DNA or the isolated DNA and/or optionally sequencing tagged RNA, the amplified cDNA or the isolated cDNA.

4. A method for DNA sequencing and RNA sequencing comprising the steps:
a) providing a sample comprising genomic DNA and RNA;
b) (i) adding at least one engineered transposome complex; and
(ii) tagging the RNA;
c) optionally amplifying tagged DNA and/or tagged RNA;
d) optionally isolating the amplified DNA and/or the amplified cDNA; and
e) sequencing tagged DNA, the amplified DNA or the isolated DNA and sequencing tagged RNA, the amplified cDNA or the isolated cDNA.

5. The method according to any one of the preceding claims, wherein the sequencing comprises single-cell sequence analysis.

6. The method according to any one of the preceding claims, wherein the engineered transposome complex comprises an oligonucleotide and an engineered transposase, preferably wherein the oligonucleotide comprises a sequencing primer site, a tagging sequence and a mosaic end.

7. The method according to claim 6, wherein the oligonucleotide comprises a 5′ phosphate group.

8. The method according to claim 6 or claim 7, wherein the engineered transposase comprises a transposase operably linked to a polypeptide that binds to a component of heterochromatin and/or euchromatin, preferably heterochromatin.

9. The method according to claim 8, wherein the polypeptide binds to methylated histone, preferably wherein the polypeptide binds to H3K9me3, H3K27me3 and/or H3K36me3.

10. The method according to claim 8 or claim 9, wherein the polypeptide binds to H3K9me3.

11. The method according to any one of claims 8-10, wherein the polypeptide comprises a chromodomain, a bromodomain, a JmJc domain, a HMG-box domain, a KRAB domain or a PWWP domain, preferably wherein the polypeptide comprises a chromodomain.

12. The method according to claim 11, wherein the chromodomain is the chromodomain of heterochromatin protein 1-α, of CBX5, of CBX8 or of yeast protein Eaf3, preferably wherein the chromodomain is the chromodomain of heterochromatin protein 1-α.

13. The method according to any one of claims 6-12, wherein the transposase is selected from Tn5, Sleeping Beauty, Tn10, Drosophila P element, bacteriophage Mu, Tc1/Mariner, IS10 and IS50, preferably wherein the transposase is Tn5.

14. The method according to any one of claims 6-13, wherein the engineered transposase comprises:
a) a sequence having at least 70% sequence identity to the sequence set forth in SEQ ID NO: 9; and/or
b) a sequence having at least 70% sequence identity to the sequence set forth in SEQ ID NO: 22 or SEQ ID NO: 24.

15. The method according to any one of claims 6-14, wherein the engineered transposase comprises a sequence having at least 70% sequence identity to the sequence set forth in SEQ ID NO: 1, SEQ ID NO: 3, SEQ ID NO: 5 or SEQ ID NO: 7.

16. The method according to any one of the preceding claims, wherein step b) further comprises adding at least one further transposome complex.

17. The method according to claim 16, wherein:
a) the at least one engineered transposome complex; and
b) the at least one further transposome complex,
each binds to a different methylated histone.

18. The method according to claim 16 or claim 17, wherein the tagging sequence of the at least one engineered transposome complex differs from the tagging sequence of the at least one further transposome complex.

19. The method according to any one of claims 16-18, wherein the signals obtained from the at least one further transposome complex and the at least one engineered transposome complex at a DNA locus are compared.

20. An engineered transposase as defined in any one of claims 8-15.

21. An engineered transposome complex as defined in any one of claims 6-15.

22. A kit comprising:
a) at least one engineered transposase according to claim 20 and at least one further transposase; or
b) at least one engineered transposome complex according to claim 21 and at least one further transposome complex.

23. Use of an engineered transposase according to claim 20 for making a DNA sequence library or libraries.

24. Use of an engineered transposome according to claim 21 for making a DNA sequence library or libraries.

25. Use of an engineered transposase according to claim 20 for DNA sequencing.

26. Use of an engineered transposome according to claim 21 for DNA sequencing.

27. Use of an engineered transposase according to claim 20 for genome and epigenetic sequencing.

28. Use of an engineered transposome according to claim 21 for genome and epigenetic sequencing.


SEQUENCE LISTING (44 sequences)

SEQ ID NO: 1 - protein, 599 aa, Artificial Sequence
  Synthetic polypeptide
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDTGSTGSTGSHMITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIC

SEQ ID NO: 2 - DNA, 1797 bp, Artificial Sequence
  Synthetic polynucleotide
atgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaatttctcaaacagtgccgatgacatcaaatctaaaaaaaagagagagcagagcaatgatacggggtcgaccggctcaactggttcccatatgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctgc

SEQ ID NO: 3 - protein, 580 aa, Artificial Sequence
  Synthetic polypeptide
MGKKTKRTADSSSSEDEEEYVVEKVLDRRVVKGQVEYLLKWKGFSEEHNTWEPEKNLDCPELISEFMKKYKKMKEGENNKPREKSESNKRKSNTGSTGSTGSHMITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIC

SEQ ID NO: 4 - DNA, 1740 bp, Artificial Sequence
  Synthetic polynucleotide
atgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaatacggggtcgaccggctcaactggttcccatatgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctgc

SEQ ID NO: 5 - protein, 586 aa, Artificial Sequence
  Synthetic polypeptide
MGKKTKRTADSSSSEDEEEYVVEKVLDRRVVKGQVEYLLKWKGFSEEHNTWEPEKNLDCPELISEFMKKYKKMKEGENNKPREKSESNKRKSNTGSTGSTGSTGSTGSHMITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIC

SEQ ID NO: 6 - DNA, 1758 bp, Artificial Sequence
  Synthetic polynucleotide
atgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaatacggggtcgaccggctcaactggttccaccgggtccacgggctcgcatatgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctgc

SEQ ID NO: 7 - protein, 605 aa, Artificial Sequence
  Synthetic polypeptide
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDTGSTGSTGSTGSTGSHMITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKIC

SEQ ID NO: 8 - DNA, 1815 bp, Artificial Sequence
  Synthetic polynucleotide
atgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaatttctcaaacagtgccgatgacatcaaatctaaaaaaaagagagagcagagcaatgatacggggtcgaccggctcaactggttccaccgggtccacgggctcgcatatgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatctgc

SEQ ID NO: 9 - protein, 476 aa, Artificial Sequence
  Synthetic polypeptide
MITSALHRAADWAKSVFSSAALGDPRRTARLVNVAAQLAKYSGKSITISSEGSKAMQEGAYRFIRNPNVSAEAIRKAGAMQTVKLAQEFPELLAIEDTTSLSYRHQVAEELGKLGSIQDKSRGWWVHSVLLLEATTFRTVGLLHQEWWMRPDDPADADEKESGKWLAAAATSRLRMGSMMSNVIAVCDREADIHAYLQDKLAHNERFVVRSKHPRKDVESGLYLYDHLKNQPELGGYQISIPQKGVVDKRGKRKNRPARKASLSLRSGRITLKQGNITLNAVLAEEINPPKGETPLKWLLLTSEPVESLAQALRVIDIYTHRWRIEEFHKAWKTGAGAERQRMEEPDNLERMVSILSFVAVRLLQLRESFTPPQALRAQGLLKEAEHVESQSAETVLTPDECQLLGYLDKGKRKRKEKAGSLQWAYMAIARLGGFMDSKRTGIASWGALWEGWEALQSKLDGFLAAKDLMAQGIKI

SEQ ID NO: 10 - DNA, 1428 bp, Artificial Sequence
  Synthetic polynucleotide
atgattaccagtgcactgcatcgtgcggcggattgggcgaaaagcgtgttttctagtgctgcgctgggtgatccgcgtcgtaccgcgcgtctggtgaatgttgcggcgcaactggccaaatatagcggcaaaagcattaccattagcagcgaaggcagcaaagccatgcaggaaggcgcgtatcgttttattcgtaatccgaacgtgagcgcggaagcgattcgtaaagcgggtgccatgcagaccgtgaaactggcccaggaatttccggaactgctggcaattgaagataccacctctctgagctatcgtcatcaggtggcggaagaactgggcaaactgggtagcattcaggataaaagccgtggttggtgggtgcatagcgtgctgctgctggaagcgaccacctttcgtaccgtgggcctgctgcatcaagaatggtggatgcgtccggatgatccggcggatgcggatgaaaaagaaagcggcaaatggctggccgctgctgcaacttcgcgtctgagaatgggcagcatgatgagcaacgtgattgcggtgtgcgatcgtgaagcggatattcatgcgtatctgcaagataaactggcccataacgaacgttttgtggtgcgtagcaaacatccgcgtaaagatgtggaaagcggcctgtatctgtatgatcacctgaaaaaccagccggaactgggcggctatcagattagcattccgcagaaaggcgtggtggataaacgtggcaaacgtaaaaaccgtccggcgcgtaaagcgagcctgagcctgcgtagcggccgtattaccctgaaacagggcaacattaccctgaacgcggtgctggccgaagaaattaatccgccgaaaggcgaaaccccgctgaaatggctgctgctgaccagcgagccggtggaaagtctggcccaagcgctgcgtgtgattgatatttatacccatcgttggcgcattgaagaatttcacaaagcgtggaaaacgggtgcgggtgcggaacgtcagcgtatggaagaaccggataacctggaacgtatggtgagcattctgagctttgtggcggtgcgtctgctgcaactgcgtgaatcttttactccgccgcaagcactgcgtgcgcagggcctgctgaaagaagcggaacacgttgaaagccagagcgcggaaaccgtgctgaccccggatgaatgccaactgctgggctatctggataaaggcaaacgcaaacgcaaagaaaaagcgggcagcctgcaatgggcgtatatggcgattgcgcgtctgggcggctttatggatagcaaacgtaccggcattgcgagctggggtgcgctgtgggaaggttgggaagcgctgcaaagcaaactggatggctttctggccgcgaaagacctgatggcgcagggcattaaaatc

SEQ ID NO: 11 - protein, 9 aa, Artificial Sequence
  Synthetic polypeptide
TGSTGSTGS

SEQ ID NO: 12 - protein, 12 aa, Artificial Sequence
  Synthetic polypeptide
TGSTGSTGSTGS

SEQ ID NO: 13 - protein, 15 aa, Artificial Sequence
  Synthetic polypeptide
TGSTGSTGSTGSTGS

SEQ ID NO: 14 - protein, 6 aa, Artificial Sequence
  Synthetic polypeptide
GGSGGS

SEQ ID NO: 15 - protein, 7 aa, Artificial Sequence
  Synthetic polypeptide
SGSGSGS

SEQ ID NO: 16 - protein, 10 aa, Artificial Sequence
  Synthetic polypeptide
GGGGSGGGGS

SEQ ID NO: 17 - protein, 10 aa, Artificial Sequence
  Synthetic polypeptide
GSGSGSGSGS

SEQ ID NO: 18 - protein, 12 aa, Artificial Sequence
  Synthetic polypeptide
GGSGGSGGSGGS

SEQ ID NO: 19 - protein, 15 aa, Artificial Sequence
  Synthetic polypeptide
GGGGSGGGGSGGGGS

SEQ ID NO: 20 - protein, 191 aa, Artificial Sequence
  Synthetic polypeptide
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSNDIARGFERGLEPEKIIGATDSCGDLMFLMKWKDTDEADLVLAKEANVKCPQIVIAFYEERLTWHAYPEDAENKEKETAKS

SEQ ID NO: 21 - DNA, 576 bp, Artificial Sequence
  Synthetic polynucleotide
atgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaatttctcaaacagtgccgatgacatcaaatctaaaaaaaagagagagcagagcaatgatatcgctcggggctttgagagaggactggaaccagaaaagatcattggggcaacagattcctgtggtgatttaatgttcctaatgaaatggaaagacacagatgaagctgacctggttcttgcaaaagaagctaatgtgaaatgtccacaaattgtgatagcattttatgaagagagactgacatggcatgcatatcctgaggatgcggaaaacaaagagaaagaaacagcaaagagctaa

SEQ ID NO: 22 - protein, 112 aa, Artificial Sequence
  Synthetic polypeptide
MGKKTKRTADSSSSEDEEEYVVEKVLDRRVVKGQVEYLLKWKGFSEEHNTWEPEKNLDCPELISEFMKKYKKMKEGENNKPREKSESNKRKSNFSNSADDIKSKKKREQSND

SEQ ID NO: 23 - DNA, 336 bp, Artificial Sequence
  Synthetic polynucleotide
atgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaatttctcaaacagtgccgatgacatcaaatctaaaaaaaagagagagcagagcaatgat

SEQ ID NO: 24 - protein, 93 aa, Artificial Sequence
  Synthetic polypeptide
MGKKTKRTADSSSSEDEEEYVVEKVLDRRVVKGQVEYLLKWKGFSEEHNTWEPEKNLDCPELISEFMKKYKKMKEGENNKPREKSESNKRKSN

SEQ ID NO: 25 - DNA, 279 bp, Artificial Sequence
  Synthetic polynucleotide
atgggaaagaaaaccaagcggacagctgacagttcttcttcagaggatgaggaggagtatgttgtggagaaggtgctagacaggcgcgtggttaagggacaagtggaatatctactgaagtggaaaggcttttctgaggagcacaatacttgggaacctgagaaaaacttggattgccctgagctaatttctgaatttatgaaaaagtataagaagatgaaggagggtgaaaataataaacccagggagaagtcagaaagtaacaagaggaaatccaat

SEQ ID NO: 26 - DNA, 19 bp, Artificial Sequence
  Synthetic polynucleotide
agatgtgtataagagacag

SEQ ID NO: 27 - DNA, 49 bp, Artificial Sequence
  Synthetic polynucleotide
tcgtcggcagcgtctccgatctcgtactagagatgtgtataagagacag

SEQ ID NO: 28 - DNA, 49 bp, Artificial Sequence
  Synthetic polynucleotide
tcgtcggcagcgtctccgatcttcctgagcagatgtgtataagagacag

SEQ ID NO: 29 - DNA, 49 bp, Artificial Sequence
  Synthetic polynucleotide
tcgtcggcagcgtctccgatcttcatgagcagatgtgtataagagacag

SEQ ID NO: 30 - DNA, 49 bp, Artificial Sequence
  Synthetic polynucleotide
tcgtcggcagcgtctccgatctcctgagatagatgtgtataagagacag

SEQ ID NO: 31 - DNA, 49 bp, Artificial Sequence
  Synthetic polynucleotide
tcgtcggcagcgtctccgatcttaaggcgaagatgtgtataagagacag

SEQ ID NO: 32 - DNA, 49 bp, Artificial Sequence
  Synthetic polynucleotide
tcgtcggcagcgtctccgatctgctacgctagatgtgtataagagacag

SEQ ID NO: 33 - DNA, 49 bp, Artificial Sequence
  Synthetic polynucleotide
tcgtcggcagcgtctccgatctaggctccgagatgtgtataagagacag

SEQ ID NO: 34 - DNA, 49 bp, Artificial Sequence
  Synthetic polynucleotide
tcgtcggcagcgtctccgatctctgcgcatagatgtgtataagagacag

SEQ ID NO: 35 - DNA, 20 bp, Artificial Sequence
  Synthetic polynucleotide
gcgccttccttacttccatg

SEQ ID NO: 36 - DNA, 20 bp, Artificial Sequence
  Synthetic polynucleotide
agtggccatctcatttccca

SEQ ID NO: 37 - DNA, 18 bp, Artificial Sequence
  Synthetic polynucleotide
aaaggccttggtcccaga

SEQ ID NO: 38 - DNA, 18 bp, Artificial Sequence
  Synthetic polynucleotide
attgaaggaacgcagccc

SEQ ID NO: 39 - DNA, 18 bp, Artificial Sequence
  Synthetic polynucleotide
gagggaggagaaagccga

SEQ ID NO: 40 - DNA, 18 bp, Artificial Sequence
  Synthetic polynucleotide
ttgtccagaccagccctt

SEQ ID NO: 41 - DNA, 22 bp, Artificial Sequence
  Synthetic polynucleotide
tcgtcggcagcgtctccgatct

SEQ ID NO: 42 - DNA, 56 bp, Artificial Sequence
  Synthetic polynucleotide
ccgggcagtgtaacacacgtccattctcgagaatggacgtgtgttacactgctttt

SEQ ID NO: 43 - DNA, 20 bp, Artificial Sequence
  Synthetic polynucleotide
agatatatataaggagacag

SEQ ID NO: 44 - DNA, 48 bp, Artificial Sequence
  Synthetic polynucleotide
  misc_feature (23)..(30): n is a, c, g, or t
tcgtcggcagcgtctccgatctnnnnnnnnagatgtgtataagagaca